(19) United States
(12) Patent Application Publication
(10) Pub. No.: US 2005/0250789 A1
(43) Pub. Date: Nov. 10, 2005

(54) Hydroxamic acid derivatives as metalloprotease inhibitors

(76) Inventors: David M. Burns, Philadelphia, PA (US); Wenqing Yao, Kennett Square, PA (US); Chunhong He, Boothwyn, PA (US)

Correspondence Address: COZEN O' CONNOR, P.C., 1900 MARKET STREET, PHILADELPHIA, PA, 19103-3508, US

(21) Appl. No.: 11/109,158

(22) Filed: Apr. 19, 2005

Related U.S. Application Data
(60) Provisional application No. 60/563,744, filed on Apr. 20, 2004.

(51) Int. Cl. A61K 31/496; A61K 31/4709; A61K 31/4545; A61K 31/452; C07D 45/14

(52) U.S. Cl. 514/253.01; 514/316; 514/318; 514/310; 544/360; 546/186; 546/194; 546/187

(57) ABSTRACT

The present invention provides compounds of Formula I or II: 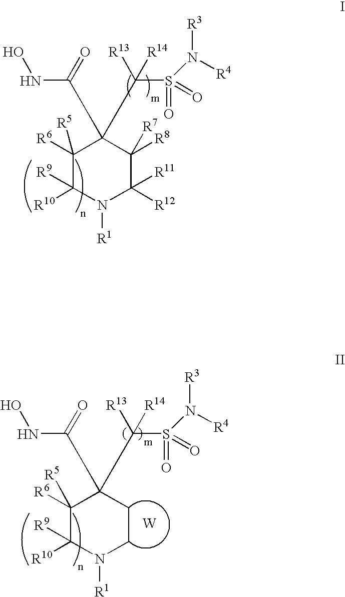 salt form or prodrug thereof, wherein variables are defined herein, that are modulators of metalloproteases such as matrix metalloproteases (MMPs) and ADAMs. The compounds or compositions described herein can be used to treat diseases associated with metalloprotease activity including, for example, arthritis, cancer, cardiovascular disorders, skin disorders, inflammation or allergic conditions.

### CROSS REFERENCE TO RELATED APPLICATIONS

This application claims the benefit of U.S. Ser. No. 60/563,744, filed Apr. 20, 2004, the disclosure of which is incorporated herein by reference in its entirety.

### FIELD OF THE INVENTION

The present invention is directed to 4-hydroxamic acid piperidines and related compounds that are inhibitors of metalloproteases. The compounds of the invention are useful in the treatment of diseases associated with metalloprotease activity.

### BACKGROUND OF THE INVENTION

Most tissues exist in a highly regulated dynamic equilibrium wherein new tissue is formed and existing tissue is degraded and eliminated. The degradation of the extracellular matrix (ECM), including connective tissue and basement membranes, is affected by the metalloproteases (or metalloproteinases) which are released from connective tissue and invading inflammatory cells. Excessive unregulated activity of these enzymes can result in undesirable tissue destruction and their activity is regulated at the transcription level, by controlled activation of the latent proenzyme and, after translation, by intracellular specific inhibitory factors such as TIMP (“Tissue Inhibitors of MetalloProteinase”) or by more general proteinase inhibitors such as α2-macroglobulins.

Several structurally related metalloproteases (MPs) are known to play an important role in the breakdown of structural proteins. These metalloproteases typically act on the intercellular matrix, and thus are involved in tissue breakdown and remodeling. There are several different families of MPs, classified by sequence homology. Several families of known MPs, as well as examples thereof, are disclosed in the art. Some MPs include matrix metalloproteases (MMPs), zinc metalloproteases, membrane bound metalloproteases, TNF converting enzymes, angiotensin-converting enzymes (ACEs), disintegrins, including ADAMs (See Wolfsberg et al, 131 J. Cell Bio. 275-78 Oct. 25, 1995), and the enkephalinases. Examples of MPs include human skin fibroblast collagenase, human skin fibroblast gelatinase, human sputum collagenase, aggrecanse and gelatinase, and human stromelysin. Collagenase, stromelysin, aggrecanase and related enzymes are thought to be important in mediating the symptomatology of a number of diseases.

Zinc proteases are typically subdivided according to the primary structure of their catalytic sites and include gluzincin, metzincin, inuzincin, carboxypeptidase, and DD carboxypeptidase subgroups (Hooper N M, 1994, FEBS Lett, 354:1-6). The metzincin subgroup is further divided into serralysins, astacins, matrixins, and adamalysins (Stocker W and Bode W, 1995, Curr Opin Struct Biol, 5:383-390).

The matrixins include the matrix metalloproteases (MMPs). MMPs constitute a family of structurally similar zinc-containing metalloproteases, which are involved in the remodeling and degradation of extracellular matrix proteins, both as part of normal physiological processes and in pathological conditions. For a review see Bode, W et al., 1996, Adv Exp Med Biol, 389:1-11. Connective tissue, extracellular matrix constituents and basement membranes are the biological materials that provide rigidity, differentiation, attachment sites and, in some cases, elasticity to biological systems. Connective tissues components include, for example, collagen, elastin, proteoglycans, fibronectin and laminin that form the scaffold for all human tissues. Under normal conditions, connective tissue turnover and/or repair processes are controlled and in equilibrium. The loss of this balance, for whatever reason, leads to a number of disease states. Inhibition of the enzymes responsible loss of equilibrium provides a control mechanism for this tissue decomposition and, therefore, a treatment for these diseases. The uncontrolled breakdown of connective tissue by metalloproteases is a feature of many pathological conditions.

Besides a role in the regulation of extracellular matrix, there is also evidence to suggest that MMPs mediate the migration of inflammatory cells into tissues (Moscatelli D and Rifkin D B, 1988, Biochim Biophys Acta, 948: 67-85). Several reports have demonstrated that various MMPs can activate a variety of important non-matrix proteins, including cytokines, chemokines, integrins, and antimicrobial peptides (see Parks W C, 2002, J Clin Invest, 110:613-4). Many of the human MMPs are over expressed in human tumors and are associated with peritumor tissue degradation and metastasis formation. Another important function of certain MMPs is to activate various enzymes, including other MMPs, by cleaving the pro-domains from their protease domains. Thus some MMPs act to regulate the activities of other MMPs, so that over-production of one MMP may lead to excessive proteolysis of extracellular matrix by another. It has also been reported that MMPs can cleave and thereby inactivate the endogenous inhibitors of other proteinases such as elastase (Winyard P G et al., 1991, FEBS Letts, 279: 91-94). Inhibitors of MMPs could thus influence the activity of other destructive proteinases by modifying the level of their endogenous inhibitors. In addition, increasing or maintaining the levels of an endogenous or administered serine protease inhibitor supports the treatment and prevention of diseases such as emphysema, pulmonary diseases, inflammatory diseases and diseases of aging such as loss of skin or organ stretch and resiliency. Thus, MMPs can be viewed as extracellular processing enzymes involved in regulating cell-cell and cell-ECM signaling events.

The adamalysins include the reprolysins, snake venom metalloproteases and the ADAMs. The ADAMs (a disintegrin and metalloprotease domain) are a family of type I transmembrane glycoproteins that are important in diverse biologic processes, such as cell adhesion and the proteolytic shedding of cell surface receptors. ADAM family members have been identified from mammalian and nonmammalian sources, including Xenopus, Drosophila, and Caenorhabditis elegans. Members of the family have a modular design, characterized by the presence of metalloprotease and integrin receptor-binding activities, and a cytoplasmic domain that in many family members specifies binding sites for various signal-transducing proteins. Members of the ADAM family have been implicated in the control of membrane fusion, cytokine, growth factor and growth factor receptor shedding, and cell migration, as well as processes such as muscle development, fertilization, neurogenesis, and cell fate determination. Loss of regulation can lead to disease and pathology. Pathologies such as infertility, inflammation and cancer have been shown to involve ADAM family members. For a review, see Wolfsberg T G and White J M, 1998, ADAM metalloproteinases. In Handbook of Proteolytic Enzymes (Barrett A J, Rawlings N D and Woessner J F eds), p. 1310-1313, Academic Press, London as well as Seals D F and Courtneidge S A, 2003, Genes and Development, 17:7-30.

Some specific examples of ADAM metalloproteases include the TNFα-converting enzyme, IL-6, TACE or ADAM17, that is currently an important target for anti-inflammatory drugs (Moss M L et al., 2001, Drug Discov Today, 6:417-426 and Black R A, 2002, Int J Biochem Cell Biol, 34:1-5). Other members of the family are also likely to be good therapeutic targets. ADAM8 has been reported to be expressed almost exclusively in cells of the immune system, particularly B-cells, monocytes, eosinophils and granulocytes. ADAM8 therefore represents a therapeutic target for human immunological-based diseases. ADAM15 is found in human aortic smooth muscle and cultured umbilical vein endothelial cells. While ADAM15 is not expressed in normal blood vessels, it has been detected in developing atherosclerotic lesions (Herren B et al., 1997, FASEB J, 11:173-180), and has also been shown to be upregulated in osteoarthritic versus normal human cartilage (Bohm B B et al., 1999, Arthritis Rheum, 42:1946-1950). Thus ADAM15 may play a role in atherosclerosis and cartilage degeneration diseases. The lymphocyte- specific expression of the ADAM28 suggests that it may have an important immunological function.

Excessive production of IgE is believed to be a major mediator of allergic responses. CD23, the low affinity receptor for IgE, is subject to ADAM type metalloprotease-dependent proteolytic release of soluble extracellular fragments, which have been shown to cause upregulation of IgE production and induction of inflammatory cytokines (see Novak N et al, 2001, Curr Opin Immunol, 13:721-726 and Mayer R J et al., 2002, Inflamm Res, 51:85-90). Increased levels of soluble CD23 have been observed in allergic asthma, in chronic B-lymphocytic leukemia and in rheumatoid arthritis. Inhibition of the enzyme(s) responsible for CD23 processing may offer a therapeutic approach for the treatment of various immune based diseases. ADAM metalloproteases also appear to be responsible for the release or shedding of soluble receptors (for example, CD30 and receptors for TNF), adhesion molecules (for example, L-selectin, ICAM-1, fibronectin), growth factors and cytokines (for example Fas ligand, TGF-α, EGF, HB-EGF, SCF IL-6, IL-1, TSH and M-CSF), and growth factor receptors (for example EGFR family members, such as Her-2 and Her-4, which have been implicated in the pathogenesis of different types of cancer (Yarden Y and Sliwkowski M X, 2001, Nature Reviews 2:127-137). For example, Her-2 is over expressed in 25-30% of human breast cancers and is associated with an increased risk of relapse and death (Slamon D J et al, 1987, Science, 235:177-182). ADAM17 has recently been shown to be involved in the regulated shedding of Her-4 (Rio C et al, 2000, J Biol Chem, 275:10379-10387). The protease responsible for Her-2 cleavage, known as Her-2 sheddase, is an unknown metalloprotease postulated to be a member of the ADAM family (Codony-Servat J et al, 1999, Cancer Res 59:1196-1201). Modulation of this activity might therefore have an important role in the modulation of human disease. For a review of the sheddase activity of ADAMs see Moss M L and Lambert M H, 2002, Essays Biochem, 38:141-153.

ADAM-TS proteases have been identified as members of the ADAM family. These proteins are novel in that they contain unique thrombospondin (TS) type I motifs in addition to some of the structurally conserved domains of other ADAM family members. The ADAM-TSs are also distinguished from the ADAMs by their lack of cysteine-rich, EGF-like, transmembrane, and cytoplasmic domains. ADAM-TS proteins have also been shown to be associated with a number of pathological or human disease states. For example, ADAM-TS-1 is a tumor-selective gene expressed in colon tumor cells and is also an inflammation-associated protein. A human ortholog of ADAM-TS-1, known as METH-1, and the related protein METH-2 have been recently shown to have antiangiogenic activity, and these or other ADAM-TS family members are believed to play important roles in regulating vascular development. ADAM-TS-2 has been implicated in the normal development of the skin. This enzyme was long known as procollagen N-proteinase, a proteinase that proteolytically removes amino peptides in the processing of type I and type II procollagens to collagens, and it was shown to be deficient in the skin of individuals with the inherited connective tissue disorder type VIIC Ehlers-Danros syndrome. ADAM-TS-4 and ADAM-TS-11 are known as aggrecanase-1 and -2 because of their ability to cleave specific sites in aggrecan, a proteoglycan that maintains the mechanical properties of cartilage. Progressive degradation and depletion of aggrecan has been implicated in degenerative joint diseases such as osteoarthritis and inflammatory joint diseases such as rheumatoid arthritis. For a review of the ADAM-TS metalloproteases see Tang B L, 2001, Int J Biochem Cell Biol, 33:33-44 and Kaushal G P and S V Shah, 2000, J Clin Invest 105:1335-1337.

The metalloproteases are one of the older classes of proteinases and are found in bacteria, fungi as well as in higher organisms. Many enzymes contain a consensus sequence, which provides two histidine ligands for the zinc whereas the third ligand is either a glutamic acid (thermolysin, neprilysin, alanyl aminopeptidase) or a histidine (astacin). Other families exhibit a distinct mode of binding of the Zn atom. Metalloproteases have therefore been isolated from a number of prokaryotic and eukaryotic sources. Acidic metalloproteases have been isolated from broad-banded copperhead and rattlesnake venoms. Neutral metalloproteases, specifically those having optimal activity at neutral pH have, for example, been isolated from Aspergillus sojae. Alkaline metalloproteases, for example, have been isolated from Pseudomonas aeruginosa and the insect pathogen Xenorhabdus luminescens. Inhibition of microbial metalloproteases may lead to growth inhibition and represent an antibiotic strategy. Inhibition of metalloproteases associated with snake venom or insect toxicity may also lead to new therapeutic strategies.

Potential therapeutic indications of MP inhibitors have been discussed in the literature. See for example, U.S. Pat. No. 6,500,847 (Bayer Corporation), U.S. Pat. No. 6,268,379 (DuPont Pharmaceuticals Company), U.S. Pat. No. 5,968,795 (Bayer Corporation), U.S. Pat. No. 5,892,112 (Glycomed Incorporated and The University of Florida), and U.S. Pat. No. 5,872,152 (British Biotech Pharmaceuticals Limited). Some examples where inhibition of metalloprotease activity would be of benefit include: a) osteoarthritis, b) rheumatic diseases and conditions such as autoimmune disease, rheumatoid arthritis, c) septic arthritis, d) cancer including tumor growth, tumor metastasis and angiogenesis, e) periodontal diseases, f) corneal, epidermal or gastric ulceration (ulcerative conditions can result in the cornea as the result of alkali burns or as a result of infection by Pseudomonas aeruginosa, Acanthamoeba, Herpes simplex and vaccinia viruses), g) proteinuria, h) various cardiovascular and pulmonary diseases such as atherosclerosis, thrombotic events, atheroma, hemodynamic shock, unstable angina, restenosis, heart failure, i) aneurysmal diseases including those of the aorta, heart or brain, j) birth control, k) dystrophobic epidermolysis bullosa, l) degenerative cartilage loss following traumatic joint injury, m) osteopenias and other diseases of abnormal bone loss including osteoporosis, n) tempero mandibular joint disease, o) pulmonary diseases such as chronic obstructive pulmonary disease, p) demyelinating diseases of the nervous system such as multiple sclerosis, q) metabolic diseases including diabetes (with enhanced collagen degradation) and obesity mediated by insulin resistance, macular degeneration and diabetic retinopathy mediated by angiogenesis, cachexia, premature skin aging, r) impaired wound healing including burns, s) decubital ulcers, t) acute and chronic neurodegenerative disorders including stroke, spinal cord and traumatic brain injury, amyotrophic lateral sclerosis, cerebral amyloid angiopathy, CNS injuries in AIDS, Parkinson's disease, Alzheimer's disease, Huntington's diseases, prion diseases, myasthenia gravis, and Duchenne's muscular dystrophy, u) pain, v) autoimmune encephalomyelitis and w) diseases linked to TNFα production and/or signaling such as a wide variety of inflammatory and/or immunomodulatory diseases, including acute rheumatic fever, rheumatoid arthritis, multiple sclerosis, allergy, periodontal diseases, hepatitis, bone resorption, sepsis, gram negative sepsis, septic shock, endotoxic shock, toxic shock syndrome, systemic inflammatory response syndrome, inflammatory bowel diseases including Crohn's disease and ulcerative colitis, Jarisch-Herxheimer reactions, asthma, adult respiratory distress syndrome, acute pulmonary fibrotic diseases, pulmonary sarcoidosis, allergic respiratory diseases, silicosis, coal worker's pneumoconiosis, alveolar injury, hepatic failure, liver disease during acute inflammation, severe alcoholic hepatitis, malaria including Plasmodium falciparum malaria and cerebral malaria, congestive heart failure, damage following heart disease, arteriosclerosis including atherosclerosis, Alzheimer's disease, acute encephalitis, brain injury, pancreatitis including systemic complications in acute pancreatitis, impaired wound healing and immune responses in infection inflammation and cancer, myelodysplastic syndromes, systemic lupus erythematosus, biliary cirrhosis, non-insulin dependent diabetes mellitus, bowel necrosis, psoriasis, cachexia and anorexia, radiation injury, and toxicity following administration of monoclonal antibodies such as OKT3, host-versus-graft reactions including ischemia reperfusion injury and allograft rejections including those of the kidney, liver, heart, and skin, lung allograft rejection including chronic lung allograft rejection (obliterative bronchitis), as well as complications due to total hip replacement, infectious diseases including Mycobacterial infection, meningitis, Helicobacter pylori infection during peptic ulcer disease, Chaga's disease resulting from Trypanosoma cruzi infection, effects of Shiga-like toxin resulting from E. coli infection, the effects of enterotoxin A resulting from Staphylococcus infection, meningococcal infection, and infections from Borrelia burgdorferi, Treponema pallidum, cytomegalovirus, influenza virus, Sendai virus, Theiler's encephalomyelitis virus, and the human immunodeficiency virus (HIV). Defective injury repair processes also occur. This can produce improper wound healing leading to weak repairs, adhesions and scarring. These latter defects can lead to disfigurement and/or permanent disabilities as with post-surgical adhesions.

Metalloprotease inhibitors are useful in treating diseases caused, at least in part, by breakdown of structural proteins. Though a variety of inhibitors have been prepared, there is a continuing need for potent metalloprotease inhibitors useful in treating such diseases. The compounds of the present invention help fulfill this and other needs.

### SUMMARY OF THE INVENTION

The present invention provides compounds of Formula I or II: 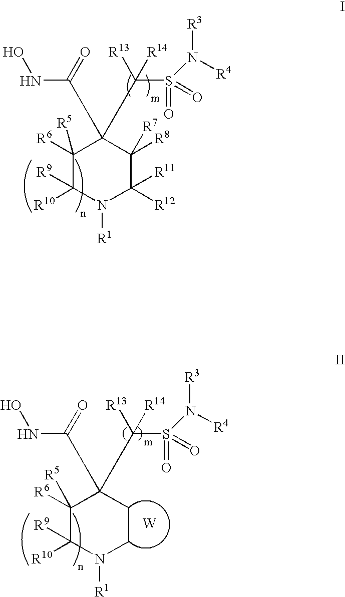
or pharmaceutically acceptable salts or prodrugs thereof, wherein constituent members are defined herein.

The present invention further provides compositions comprising a compound of Formula I or II and a pharmaceutically acceptable carrier.

The present invention further provides methods for modulating activity of a metalloprotease comprising contacting the metalloprotease with a compound of Formula I or II.

The present invention further provides methods for treating a disease associated with metalloprotease activity in a mammalian subject, the method comprising administering to the mammalian subject a therapeutically effective amount of a compound of Formula I or II.

The present invention further provides methods for treating a disease associated with activity of a Her-2 sheddase, a growth factor sheddase, or a cytokine sheddase in a mammalian subject, the method comprising administering to the mammalian subject a therapeutically effective amount of a compound of Formula I or II.

The present invention further provides methods for treating a disease associated with ADAM activity in a mammalian subject, the method comprising administering to the mammalian subject a therapeutically effective amount of a compound of Formula I or II.

The present invention further provides methods for treating a disease associated with MMP activity in a mammalian subject, the method comprising administering to the mammalian subject a therapeutically effective amount of a compound of Formula I or II.

### DETAILED DESCRIPTION

Compounds

The present invention provides, inter alia, compounds and pharmaceutically acceptable salts thereof that modulate activity of metalloproteases and are useful in treating diseases or other conditions associated with abnormal metalloprotease activity. Compounds of the invention include compounds of Formula I or II: 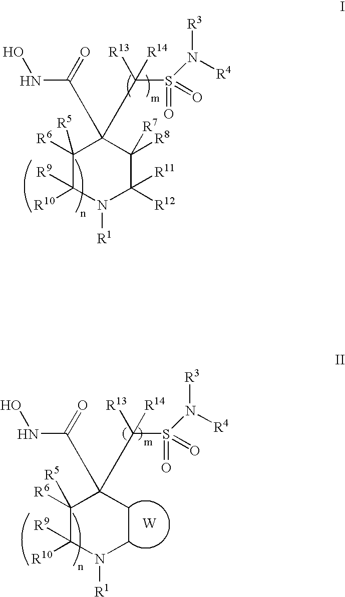
or pharmaceutically acceptable salt or prodrug thereof, wherein:
R¹ is: 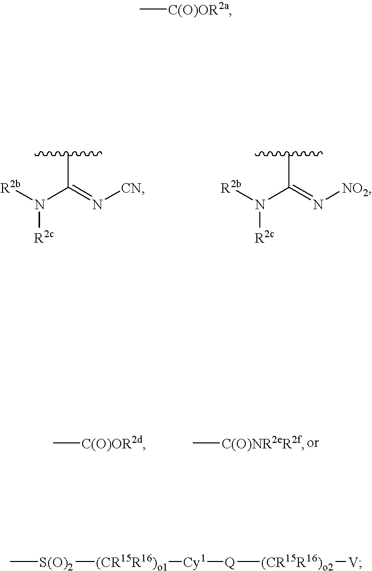
R^{2a} is:
   C₂₋₁₀ alkenyl;
   C₂₋₁₀ alkynyl;
   —(CR¹⁷R¹⁸)ₚ₁—X—(CR¹⁷R¹⁸)ₚ₂—Y;
   —(CR¹⁷R¹⁸)_{q1}—NR^{A}R^{B};
   —(CR¹⁷R¹⁸)_{q2}-Z;
   carbocyclyl substituted by at least one OH or C₁₋₈ haloalkoxy; or
   heterocyclyl optionally substituted by 1, 2 or 3 R^{a};
R^{2b} and R^{2c} are each, independently, H, C₁₋₆ alkyl, carbocyclyl, heterocyclyl, carbocyclylalkyl or heterocyclylalkyl, each optionally substituted by 1 or 2 R^{b};
or R^{2b} and R^{2c} together with the N atom to which they are attached form a 4-14 membered heterocyclyl group optionally substituted by 1 or 2 R^{b};
R^{2d} is C₁₋₈ alkyl, carbocyclyl or heterocyclyl, each substituted by 1, 2 or 3 C₁₋₈ haloalkoxy;
R^{2e} is C₁₋₈ alkyl, carbocyclyl or heterocyclyl, each substituted by 1, 2 or 3 C₁₋₈ haloalkoxy;
R^{2f} is H, C₁₋₈ alkyl, C₃₋₇ cycloalkyl or heterocycloalkyl, wherein said C₁₋₈ alkyl, C₃₋₇ cycloalkyl or heterocycloalkyl is optionally substituted by 1 or 2 R^{c};
or R^{2e} and R^{2f} together with the N atom to which they are attached form a heterocyclyl group substituted by at least one C₁₋₈ haloalkoxy and optionally substituted by 1 or 2 R^{c};
R³ and R⁴ are each, independently, H, C₁₋₈ alkyl, C₃₋₇ cycloalkyl or heterocycloalkyl, wherein each of said C₁₋₈ alkyl, C₃₋₇ cycloalkyl or heterocycloalkyl is optionally substituted by 1 or 2 R^{d};
or R³ and R⁴ together with the N atom to which they are attached form a heterocyclyl group optionally substituted with -L-Cy² and optionally substituted with 1, 2, 3, 4 or 5 R^{e};
R⁵, R⁶, R⁷, R⁸, R⁹, R¹⁰, R¹¹ and R¹² are each, independently, H, halo, CN, NO₂, C₁₋₄ alkyl, C₁₋₄ haloalkyl, C(O)R^{w}, C(O)OR^{x}, OR^{x}, OC(O)R^{w}, NR^{y}C(O)R^{w}, SOR^{w}, SO₂R^{w} or NR^{y}R^{z};
or R⁵ and R⁶, R⁷ and R⁸, R⁹ and R¹⁰, or R¹¹ and R¹² together with the C atom to which they are attached form C═O, C═C(R¹⁹R²⁰) or a C₃₋₇ cycloalkyl or 3-7 membered heterocycloalkyl group, wherein said C₃₋₇ cycloalkyl or 3-7 membered heterocycloalkyl group is optionally substituted by 1 or 2 R^{f};
R¹³ and R¹⁴ are each, independently, H, OH, halo, C₁₋₄ alkyl, C₁₋₄ haloalkyl, C₁₋₄ alkoxy, C₁₋₄ haloalkoxy, carbocyclyl, carbocyclylalkyl, heterocyclyl or heterocyclylalkyl, wherein each of said carbocyclyl, carbocyclylalkyl, heterocyclyl or heterocyclylalkyl is optionally substituted by one or more halo, C₁₋₄ alkyl, C₁₋₄ haloalkyl, OH, C₁₋₄ alkoxy, C₁₋₄ haloalkoxy, COOH, COO(C₁₋₄ alkyl), NH₂, NH(C₁₋₄ alkyl) or N(C₁₋₄ alkyl)₂;
or R¹³ and R¹⁴ together with the C atom to which they are attached form CO, C═C(R²¹R²²), C₃₋₇ cycloalkyl or 3-7 membered heterocycloalkyl group, wherein said C₃₋₇ cycloalkyl or 3-7 membered heterocycloalkyl group is optionally substituted with 1 or 2 R^{f};
R¹⁵ and R¹⁶ are each, independently, H, halo, C₁₋₄ alkyl, C₁₋₄ haloalkyl, C₁₋₄ alkoxy or C₁₋₄ haloalkoxy, carbocyclyl, carbocyclylalkyl, heterocyclyl or heterocyclylalkyl, wherein each of said carbocyclyl, carbocyclylalkyl, heterocyclyl or heterocyclylalkyl is optionally substituted by one or more halo, C₁₋₄ alkyl, C₁₋₄ haloalkyl, OH, C₁₋₄ alkoxy, C₁₋₄ haloalkoxy, COOH, COO(C₁₋₄ alkyl), NH₂, NH(C₁₋₄ alkyl) or N(C₁₋₄ alkyl)₂;
or R¹⁵ and R¹⁶ together with the C atom to which they are attached form a 3- to 7-membered cycloalkyl or heterocycloalkyl group which is optionally substituted by one or more halo, C₁₋₄ alkyl, C₁₋₄ haloalkyl, OH, C₁₋₄ alkoxy, C₁₋₄ haloalkoxy, NH₂, COOH, COO(C₁₋₄ alkyl), NH(C₁₋₄ alkyl) or N(C₁₋₄ alkyl)₂;
R¹⁷ and R¹⁸ are each, independently, H, halo, C₁₋₄ alkyl, C₁₋₄ haloalkyl, C₁₋₄ alkoxy or C₁₋₄ haloalkoxy, carbocyclyl, carbocyclylalkyl, heterocyclyl or heterocyclylalkyl, wherein each of said carbocyclyl, carbocyclylalkyl, heterocyclyl or heterocyclylalkyl is optionally substituted by one or more halo, C₁₋₄ alkyl, C₁₋₄ haloalkyl, OH, C₁₋₄ alkoxy, C₁₋₄ haloalkoxy, COOH, COO(C₁₋₄ alkyl), NH₂, NH(C₁₋₄ alkyl) or N(C₁₋₄ alkyl)₂;
or R¹⁷ and R¹⁸ together with the C atom to which they are attached form a 3- to 7-membered cycloalkyl or heterocycloalkyl group which is optionally substituted by one or more halo, C₁₋₄ alkyl, C₁₋₄ haloalkyl, OH, C₁₋₄ alkoxy, C₁₋₄ haloalkoxy, NH₂, COOH, COO(C₁₋₄ alkyl), NH(C₁₋₄ alkyl) or N(C₁₋₄ alkyl)₂;
R¹⁹, R²⁰, R²¹, and R²² are each, independently, H, C₁₋₆ alkyl, C₂₋₆ alkenyl, C₂₋₆ alkynyl, carbocyclyl, carbocyclylalkyl, heterocyclyl, heterocyclylalkyl, CO—(C₁₋₄ alkyl), CO-(carbocyclyl), CO-(carbocyclylalkyl), or COO—(C₁₋₄ alkyl);
R^{A} and R^{B} are each, independently, H, C₁₋₆ alkyl, C₂₋₆ alkenyl, C₂₋₆ alkynyl, carbocyclyl, carbocyclylalkyl, heterocyclyl, heterocyclylalkyl, CO—(C₁₋₄ alkyl), CO-(carbocyclyl), CO-(carbocyclylalkyl), SO₂—(C₁₋₄ alkyl), SO₂-(carbocyclyl) or SO₂-(carbocyclylalkyl);
or R^{A} and R^{B} together with the N atom to which they are attached form a 3-, 4-, 5-, 6- or 7-membered heterocycloalkyl group optionally substituted by 1, 2 or 3 R^{g};
Cy¹ is absent, carbocyclyl or heterocyclyl, wherein said carbocyclyl or heterocyclyl is optionally substituted by 1, 2, 3, 4 or 5 R^{h};
CY² is carbocyclyl or heterocyclyl, wherein said carbocyclyl or heterocyclyl is optionally substituted by 1, 2, 3, 4 or 5 Rⁱ;
L is absent, O, S, CO, C(O)O, OC(O), NRⁿ, NRⁿS(O)ᵣ, NRⁿC(O), NRⁿC(O)O, NRⁿC(O)NRⁿ, S(O)ᵣNRⁿ, NRⁿS(O)ᵣ, NRⁿS(O)NRⁿ, C₁₋₁₀ alkylene substituted with one or more R^{m} or C₂₋₁₀ alkenylene substituted with one or more R^{m};
Q is O, S or NR^{o};
V is H, carbocyclyl or heterocyclyl, wherein said carbocyclyl or heterocyclyl is optionally substituted by 1, 2, 3, 4 or 5 R^{j};
W is fused carbocyclyl or fused heterocyclyl;
X is O or S;
Y is H, halo, carbocyclyl or heterocyclyl, wherein said carbocyclyl or heterocyclyl is optionally substituted by 1, 2, 3, 4 or 5 R^{k};
Z is a 3-, 4-, 5-, 6- or 7-membered heterocyclyl group optionally substituted by 1, 2, 3, 4 or 5 R^{L};
R^{a}, R^{b}, R^{c}, R^{d}, R^{e}, R^{f}, R^{g}, R^{h}, Rⁱ, R^{j}, R^{k}, R^{L} and R^{wc} are each, independently T, (C₁₋₈ alkylene)-T, (C₂₋₈ alkenylene)-T, (C₂₋₈ alkynylene)-T, C(O)NR^{aa}(CR^{bb}R^{cc})ₛ-T, C(O)O(CR^{bb}R^{cc})ₛ-T, S(O)ₜ(CR^{bb}R^{cc})ₛ-T, (CR^{bb}R^{cc})ₛ—O—(CR^{bb}R^{cc})ₛ-T, OH, Cl, F. Br, I, CN, NO₂, NR^{I}R^{II}, COR^{III}, COOR^{IV}, OR^{IV}, CONR^{I}R^{II}, NR^{I}CONR^{I}R^{II}, OCONR^{I}R^{II}, NR^{I}COR^{II}, SO₂NR^{I}R^{II}, NR^{I}SO₂R^{II}, NR^{I}SO₂NR^{I}R^{II}, OSO₂NR^{I}R^{II}, S(O)ₜR^{V}, C₁₋₈ haloalkyl, carbocyclyl, heterocyclyl, carbocyclylalkyl, or heterocyclylalkyl, wherein each of said carbocyclyl, heterocyclyl, carbocyclylalkyl, and heterocylcylalkyl groups is optionally substituted by one or more C₁₋₈ alkyl, alkoxy, halo, haloalkyl, haloalkoxy, cyano, nitro, amino, alkylamino, dialkylamino, carboxy, carboxy alkyl ester, carboxy aryl ester, aminocarbonyl, alkylaminocarbonyl, dialkylaminocarbonyl, sulfonyl, aminosulfonyl, alkylaminosulfonyl, dialkylaminosulfonyl, arylsulfonyl, arylsulfinyl, alkylsulfonyl or arylsufonyl;
R^{m} is halo, C₁₋₄ alkyl, C₁₋₄ haloalkyl, OH, C₁₋₄ alkoxy, C₁₋₄ haloalkoxy, NH₂, COOH, COO(C₁₋₄ alkyl), NH(C₁₋₄ alkyl) or N(C₁₋₄ alkyl)₂;
Rⁿ is H or C₁₋₄ alkyl;
R^{o} is H, C₁₋₄ alkyl, carbocyclyl, heterocyclyl, carbocyclylalkyl, heterocyclylalkyl, C(O)—(C₁₋₄ alkyl) or C(O)-(cycloalkyl);
R^{w} is H, halo, C₁₋₄ alkyl, C₁₋₄ haloalkyl or NR^{wa}R^{wb};
R^{x} is C₁₋₄ alkyl or C₁₋₄ haloalkyl;
R^{y} and R^{z} are each, independently, H or C₁₋₄ alkyl;
or R^{y} and R^{z} together with the N atom to which they are attached form a 5-, 6-, or 7-membered heterocyclyl group;
R^{wa} and R^{wb} are each, independently, H, C₁₋₆ alkyl, carbocyclyl, heterocyclyl, carbocyclylalkyl or heterocyclylalkyl, each optionally substituted by 1 or 2 R^{wc};
or R^{wa} and R^{wb} together with the N atom to which they are attached form a 4-14 membered heterocyclyl group optionally substituted by 1 or 2 R^{wc};
T is H, C₁₋₁₀ alkyl optionally substituted with 1, 2, 3, 4 or 5 R^{bb}; C₂₋₁₀ alkenyl optionally substituted with 1, 2, 3, 4 or 5 R^{bb}, C₂₋₁₀ alkynyl optionally substituted with 1, 2, 3, 4 or 5 R^{bb}, carbocyclyl optionally substituted with 1, 2, 3, 4 or 5 R^{bb} or heterocyclyl optionally substituted with 1, 2, 3, 4 or R^{bb};
R^{aa}, R^{bb} and R^{cc} are each, independently, H, C₁₋₆ alkyl, C₂₋₆ alkenyl, C₂₋₆ alkynyl, OH, Cl, F, Br, I, CN, NO₂, NR^{I}R^{II}, COR^{III}, COOR^{IV}, OR^{IV}, CONR^{I}R^{II}, R^{I}NCONR^{I}R^{II}, OCONR^{I}R^{II}, R^{I}NCOR^{II}, SO₂NR^{I}R^{II}, NR^{I}SO₂R^{II}, NR^{I}SO₂NR^{I}R^{II}, OSO₂NR^{I}R^{II}, SOₚR^{V}, C₁₋₈ haloalkyl, carbocyclyl, heterocyclyl, carbocyclylalkyl, heterocyclylalkyl, carbocyclyloxy or heterocyclyloxy, wherein each of said carbocyclyl, heterocyclyl, carbocyclylalkyl, heterocyclylalkyl, carbocyclyloxy or heterocyclyloxy group is optionally substituted by one or more C₁₋₈ alkyl, alkoxy, halo, C₁₋₄ haloalkyl, C₁₋₈ haloalkoxy, cyano, nitro, amino, alkylamino, dialkylamino, carboxy, carboxy alkyl ester, carboxy aryl ester, aminocarbonyl, alkylaminocarbonyl, dialkylaminocarbonyl, sulfonyl, aminosulfonyl, alkylaminosulfonyl, dialkylaminosulfonyl, arylsulfonyl, arylsulfinyl, alkylsulfonyl or arylsufonyl;
R^{I} and R^{II} are each, independently, H, C₁₋₆ alkyl or carbocyclyl;
R^{III} and R^{IV} are each, independently, H, C₁₋₆ alkyl, haloalkyl, carbocyclyl, heterocyclyl, carbocyclylalkyl or heterocyclylalkyl, wherein said carbocyclyl, heterocyclyl, carbocyclylalkyl or heterocyclylalkyl are each optionally substituted by one or more halo, C₁₋₄ alkyl or C₁₋₄ alkoxy;
R^{V} is C₁₋₆ alkyl, haloalkyl, carbocyclyl or heterocyclyl;
m is 1 or 2;
n is 0, 1 or 2;
o1 is 0, 1, 2, 3, 4, 5 or 6;
o2 is 0, 1, 2, 3, 4, 5 or 6;
p1 is 1, 2, 3, 4, 5 or 6;
p2 is 1, 2, 3, 4, 5 or 6;
q1 is 1, 2, 3, 4, 5 or 6;
q2 is 1, 2, 3, 4, 5 or 6;
r is 1 or 2;
s is 1, 2, 3, 4, 5 or 6; and
t is 1 or 2.

In some embodiments, R¹ is —C(O)OR^{2a}.

In some embodiments, R^{2a} is C₂₋₁₀ alkenyl or C₂₋₁₀ alkynyl.

In some embodiments, R^{2a} is —(CR¹⁷R¹⁸)ₚ₁—X—(CR¹⁷R¹⁸)ₚ₂—Y.

In some embodiments, R^{2a} is —(CR¹⁷R¹⁸)ₚ₁—O—(CR¹⁷R¹⁸)ₚ₂—Y.

In some embodiments, Y is H, carbocyclyl or heterocyclyl.

In some embodiments, Y is H or aryl.

In some embodiments, Y is H or phenyl.

In some embodiments, p1 is 1 or 2.

In some embodiments, p2 is 1 or 2.

In some embodiments, R^{2a} is —(CR¹⁷R¹⁸)_{q1}—NR^{A}R^{B}.

In some embodiments, R^{A} and R^{B} are each, independently, H, C₁₋₆ alkyl or CO—(C₁₋₄ alkyl).

In some embodiments, R^{A} and R^{B} are each, independently, H, methyl, ethyl, n-propyl, isopropyl, CO-methyl, CO-ethyl, CO-(n-propyl) or CO-(isopropyl).

In some embodiments, at least one of R^{A} and R^{B} is other than H.

In some embodiments, R^{A} and R^{B} together with the N atom to which they are attached form a 3-, 4-, 5-, 6- or 7-membered heterocycloalkyl group optionally substituted by Cl, F, Br, I, C₁₋₄ alkyl, C₁₋₄ haloalkyl, C₁₋₄ alkoxy, C₁₋₄ haloalkoxy, hydroxy, COOH, COO(C₁₋₄ alkyl), NH₂, NH(C₁₋₄ alkyl) or N(C₁₋₄ alkyl)₂.

In some embodiments, q1 is 1 or 2.

In some embodiments, wherein q1 is 1.

In some embodiments, R^{2a} is —(CR¹⁷R¹⁸)_{q2}-Z.

In some embodiments, Z is a 3-, 4-, 5-, 6- or 7-membered heterocycloalkyl group optionally substituted by 1, 2, 3, 4 or 5 R^{L}.

In some embodiments, is a 3-, 4-, 5-, 6- or 7-membered heterocycloalkyl group optionally substituted by 1, 2, 3, 4 or 5 R^{L}, wherein said heterocycloalkyl group contains at least one ring-forming N atom.

In some embodiments, Z is aziridinyl, azetidinyl, pyrrolidinyl, piperidinyl or azepanyl.

In some embodiments, q2 is 1 or 2.

In some embodiments, q2 is 1.

In some embodiments, R^{2a} is carbocyclyl substituted by at least one OH or C₁₋₈ haloalkoxy. In further embodiments, the carbocyclcyl can be aryl, such as phenyl.

In some embodiments, R^{2a} is carbocyclyl substituted by at least one C₁₋₄ haloalkoxy.

In some embodiments, R^{2a} is carbocyclyl substituted by at least one OCF₃ or OCF₂CF₃.

In some embodiments, R^{2a} is aryl substituted by at least one OCF₃ or OCF₂CF₃.

In some embodiments, R^{2a} is heterocyclyl optionally substituted by 1, 2 or 3 R^{a}.

In some embodiments, R^{2a} is heterocycloalkyl optionally substituted by one or more Cl, F, Br, I, C₁₋₈ alkyl, C₁₋₈ haloalkyl, OH, C₁₋₈ alkoxy, C₁₋₈ haloalkoxy, CN, NO₂, NH₂, COOH, COO(C₁₋₄ alkyl), NH(C₁₋₄ alkyl) or N(C₁₋₄ alkyl)₂.

In some embodiments, R^{2a} is a heterocycloalkyl group comprising at least one ring-forming O atom.

In some embodiments, R^{2a} is a heterocycloalkyl group comprising at least one ring-forming N atom.

In some embodiments, R^{2a} is oxiranyl, oxetanyl, tetrahydrofuranyl, tetrahydropyranyl or oxepanyl.

In some embodiments, R^{2a} is aziridinyl, azetidinyl, pyrrolidinyl, piperidinyl or azepanyl.

In some embodiments, R¹ is

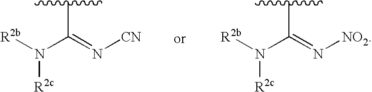

In some embodiments, R^{b} and R^{2c} are each, independently, H, C₁₋₆ alkyl, carbocyclyl, heterocyclyl, carbocyclylalkyl or heterocyclylalkyl, each optionally substituted by 1 or 2 R^{b}.

In some embodiments, R^{2b} and R^{2c} are each, independently, H or C₁₋₆ alkyl.

In some embodiments, R^{2b} and R^{2c} are each, independently, methyl, ethyl, n-propyl, isopropyl, n-butyl, isobutyl or t-butyl.

In some embodiments, at least one of R^{2b} and R^{2c} is other then H.

In some embodiments, at least one of R^{2b} and R^{2c} are other than H.

In some embodiments, R^{2b} and R^{2c} together with the N atom to which they are attached form a 4-14 membered heterocyclyl group optionally substituted by 1 or 2 R^{b}.

In some embodiments, R^{2b} and R^{2c} together with the N atom to which they are attached form a 4-, 5-, 6- or 7-membered heterocycloalkyl group optionally substituted by one or more Cl, F, Br, I, CN, NO₂, C₁₋₄ alkyl or C₁₋₄ haloalkyl.

In some embodiments, R^{2b} and R^{2c} together with the N atom to which they are attached form aziridinyl, azetidinyl, pyrrolidinyl, piperidinyl, azepanyl, piperazinyl, morpholino, 2,5-dihydro-1H-pyrrolyl, 2,3-dihydro-1H-pyrrolyl, 1,2,3,6-tetrahydropyridinyl or 1,2,3,4-tetrahydropyridinyl, each of which is optionally substituted by 1 or 2 R^{b}.

In some embodiments, R^{2b} and R^{2c} together with the N atom to which they are attached form a 5- or 6-membered heterocycloalkyl group optionally substituted by one or more Cl, F, Br, I, C₁₋₄ alkyl, C₁₋₄ haloalkyl or aryl.

In some embodiments, R¹ is —C(O)R^{2d}, —C(O)NR^{2e}R^{2f} or —S(O)₂—(CR¹⁵R¹⁶)ₒ₁-Cy¹-Q-(CR¹⁵R¹⁶)ₒ₂—V.

In some embodiments, R³ and R⁴ together with the N atom to which they are attached form a heterocyclyl group optionally substituted with at least one -L-Cy² and optionally substituted with 1, 2, 3, 4 or 5 R^{e}.

In some embodiments, R³ and R⁴ together with the N atom to which they are attached form a heterocycloalkyl group optionally substituted with at least one -L-Cy² and optionally substituted with 1, 2, 3, 4 or 5 R^{e}.

In some embodiments, R³ and R⁴ together with the N atom to which they are attached form aziridinyl, azetidinyl, pyrrolidinyl, piperidinyl, azepanyl, piperazinyl, morpholino, 2,5-dihydro-1H-pyrrolyl, 2,3-dihydro-1H-pyrrolyl, 1,2,3,6-tetrahydropyridinyl, 1,2,3,4-tetrahydropyridinyl, 2,3-dihydro-1H-indolyl, 2,3-dihydro-1H-isoindolyl, 1,2,3,4-tetrahydroquinolyl or 1,2,3,4-tetrahydro-isoquinolyl each optionally substituted with at least one -L-Cy² and optionally substituted with 1, 2, 3, 4 or 5 R^{e}.

In some embodiments, R³ and R⁴ together with the N atom to which they are attached form a 5- or 6-membered heterocycloalkyl group substituted with at least one -L-Cy² and optionally substituted with 1, 2, 3, 4 or 5 R^{e}.

In some embodiments, L is O, CH₂ or absent.

In some embodiments, L is absent.

In some embodiments, Cy² is aryl or heteroaryl each optionally substituted by 1, 2, 3, 4 or 5 Rⁱ.

In some embodiments, Cy² is aryl or heteroaryl each optionally substituted by 1, 2, 3, 4 or 5 C₁₋₄ alkyl, C₁₋₄ haloalkyl, Cl, F, Br, I, CN, NO₂, amino or aminoalkyl.

In some embodiments, m is 1.

In some embodiments, R⁵, R⁶, R⁷, R⁸, R⁹, R¹⁰, R¹¹ and R¹² are each, independently, H, halo, CN, NO₂, C₁₋₄ alkyl or C₁-₄ haloalkyl.

In some embodiments, R⁵, R⁶, R⁷, R⁸, R⁹, R¹⁰, R¹¹ and R¹² are each H.

In some embodiments, R¹³ and R¹⁴ are each H.

In some embodiments, n is 1.

In some embodiments, the compounds have Formula I.

In some embodiments, the compounds have Formula II.

In some embodiments, the compounds have Formula III:

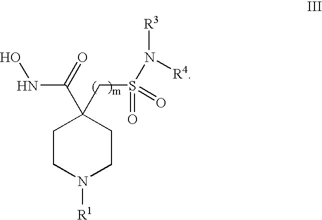

In some embodiments, the compound has Formula III and:
R¹ is —C(O)OR^{2a};
m is 1; and
R³ and R⁴ together with the N atom to which they are attached form a heterocycloalkyl group optionally substituted with at least one -Cy² and optionally substituted with 1, 2, or 3 R^{e}.

In some embodiments, the compound has Formula III and:
R¹ is 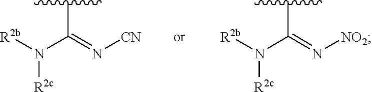
m is 1; and
R³ and R⁴ together with the N atom to which they are attached form a heterocycloalkyl group optionally substituted with at least one -Cy² and optionally substituted with 1, 2, or 3 R^{e}.

In some embodiments, the compound has Formula III and:
R¹ is —C(O)R^{2d};
m is 1; and
R³ and R⁴ together with the N atom to which they are attached form a heterocycloalkyl group optionally substituted with at least one -Cy² and optionally substituted with 1, 2, or 3 R^{e}.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, cyclohexenyl, and the like.

As used herein, “alkynyl” refers to an alkyl group having one or more triple carbon-carbon bonds. Example alkynyl groups include ethynyl, propynyl, and the like.

As used herein, “alkylene” or “alkylenyl” refers to a bivalent alkyl group. An example alkylene group is methylene or ethylene.

As used herein, “alkenylene” or “alkenylenyl” refers to a bivalent alkenyl group.

As used herein, “haloalkyl” refers to an alkyl group having one or more halogen substituents. Example haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.”

As used herein, “carbocyclyl” groups are saturated (i.e., containing no double or triple bonds) or unsaturated (i.e., containing one or more double or triple bonds) cyclic hydrocarbon moieties. Carbocyclyl groups can be mono-, poly- (e.g., 2, 3 or 4 fused rings) or spirocyclic. Example carbocyclyl groups include cyclopropyl, cyclobutyl, cyclopentyl, cyclohexyl, cycloheptyl, cyclopentenyl, 1,3-cyclopentadienyl, cyclohexenyl, norbornyl, norpinyl, norcarnyl, adamantyl, phenyl, and the like. Carbocyclyl groups can be aromatic (e.g., “aryl”) or non-aromatic (e.g., “cycloalkyl”). In some embodiments, carbocyclyl groups can have from about 3 to about 30 carbon atoms, about 3 to about 20, about 3 to about 10, or about 3 to about 7 ring-forming carbon atoms.

As used herein, “aryl” refers to an aromatic carbocyclyl group including monocyclic or polycyclic aromatic hydrocarbons such as, for example, phenyl, naphthyl, anthracenyl, phenanthrenyl, indanyl, indenyl, and the like. In some embodiments, aryl groups have from 6 to about 20 ring-forming carbon atoms.

As used herein, “cycloalkyl” refers to non-aromatic carbocyclyl groups including cyclized alkyl, alkenyl, and alkynyl groups. Cycloalkyl groups can include bi- or poly-cyclic (e.g., 2, 3, or 4 fused rings) ring systems as well as spiro ring systems. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of pentane, pentene, hexane, and the like. In some embodiments, cycloalkyl groups can have from 3 to about 30 carbon atoms, about 3 to about 20, about 3 to about 10, or about 3 to about 7 ring-forming carbon atoms. In some embodiments, the cycloalkyl group has from 0 to 3 double or 0 to 2 triple ring-forming bonds.

As used herein, “heterocyclyl” or “heterocycle” refers to a carbocyclyl group wherein one or more of the ring-forming carbon atoms of the carbocyclyl group is replaced by a heteroatom such as O, S, or N. Heterocyclyl groups can be aromatic (e.g., “heteroaryl”) or non-aromatic (e.g., “heterocycloalkyl”). Heterocyclyl groups can correspond to hydrogenated and partially hydrogenated heteroaryl groups. Heterocyclyl groups can be characterized as having 3 to about 14, 4 to about 14, or 3 to about 7 ring-forming atoms. In some embodiments, heterocyclyl groups can contain, in addition to at least one heteroatom, from about 1 to about 20, about 2 to about 10, or about 2 to about 7 carbon atoms and can be attached through a carbon atom or heteroatom. In further embodiments, the heteroatom can be oxidized (e.g., an oxo or sulfido substituent) or an nitrogen atom can be quataernized. Examples of heterocyclyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like, as well as any of the groups listed below for “heteroaryl” and “heterocycloalkyl.” Further example heterocycles include pyrimidinyl, phenanthridinyl, phenanthrolinyl, phenazinyl, phenothiazinyl, phenoxathiinyl, phenoxazinyl, phthalazinyl, piperazinyl, piperidinyl, 3,6-dihydropyridyl, 1,2,3,6-tetrahydropyridyl, 1,2,5,6-tetrahydropyridyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, tetrahydrofuranyl, tetrahydroisoquinolinyl, tetrahydroquinolinyl, tetrazolyl, 6H-1,2,5-thia-diazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, octahydro-isoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, quinazolinyl, quinolinyl, 4H-quinolizinyl, quinoxalinyl, quinuclidinyl, acridinyl, azocinyl, benzimidazolyl, benzofuranyl, benzothiofuranyl, benzo-thiophenyl, benzoxazolyl, benzthiazolyl, benztriazolyl, benztetrazolyl, benzisoxazolyl, benzisothiazolyl, benzimidazolinyl, methylenedioxyphenyl, morpholinyl, naphthyridinyl, deca-hydroquinolinyl, 2H,6H-1,5,2dithiazinyl, dihydrofuro[2,3-b]tetrahydrofuran, furanyl, furazanyl, carbazolyl, 4aH-carbazolyl, carbolinyl, chromanyl, chromenyl, cinnolinyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl and isoxazolyl. Further examples of heterocycles include azetidin-1-yl, 2,5-dihydro-1H-pyrrol-1-yl, piperindin-1-yl, piperazin-1-yl, pyrrolidin-1-yl, isoquinol-2-yl, pyridin-1-yl, 3,6-dihydropyridin-1-yl, 2,3-dihydroindol-1-yl, 1,3,4,9-tetrahydrocarbolin-2-yl, thieno[2,3-c]pyridin-6-yl, 3,4,10,10a-tetrahydro-1H-pyrazino[1,2-a]indol-2-yl, 1,2,4,4a,5,6-hexahydro-pyrazino[1,2-a]quinolin-3-yl, pyrazino[1,2-a]quinolin-3-yl, diazepan-1-yl, 1,4,5,6-tetrahydro-2H-benzo[f]isoquinolin-3-yl, 1,4,4a,5,6,10b-hexahydro-2H-benzo[f]isoquinolin-3-yl, 3,3a,8,8a-tetrahydro-1H-2-aza-cyclopenta[a]inden-2-yl, and 2,3,4,7-tetrahydro-1H-azepin-1-yl, azepan-1-yl. Also included are fused ring (such as benzo-fused) and spiro compounds containing, for example, the above heterocycles.

As used herein, “heteroaryl” groups are aromatic heterocyclyl groups and include monocyclic and polycyclic (e.g., 2, 3, or 4 fused rings) aromatic hydrocarbons that have at least one heteroatom ring member such as sulfur, oxygen, or nitrogen. Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl indazolyl, 1,2,4-thiadiazolyl, isothiazolyl, benzothienyl, purinyl, carbazolyl, benzimidazolyl, 2,3-dihydrobenzofuranyl, 2,3-dihydrobenzothienyl, 2,3-dihydrobenzothienyl-S-oxide, 2,3-dihydrobenzothienyl-S-dioxide, and the like. In some embodiments, heteroaryl groups can have from 1 to about 20 carbon atoms, and in further embodiments from about 3 to about 20, 3 to about 14 or 4 to about 14 ring-forming atoms. In some embodiments, heteroaryl groups have 1 to about 4, 1 to about 3, or 1 to 2 ring forming heteroatoms.

As used herein, “heterocycloalkyl” refers to non-aromatic heterocyclyl groups including cyclized alkyl, alkenyl, and alkynyl groups where one or more of the ring-forming carbon atoms is replaced by a heteroatom such as an O, N, or S atom. In some embodiments, heterocycloalkyl groups can have from 2 to about 30 carbon atoms, about 2 to about 20, about 2 to about 10, or about 2 to about 7 ring-forming carbon atoms in addition to at least one ring-forming heteroatom. In some embodiments, the heterocycloalkyl group has from 0 to 3 double or 0 to 2 triple ring-forming bonds. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of saturated heterocycles such as indolene and isoindolene groups. Example heterocycloalkyl groups include morpholino, thiomorpholino, piperazinyl, tetrahydrofuranyl, tetrahydrothienyl, 1,3-dihydroisoindolyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “aryloxy” refers to an —O-aryl group. An example aryloxy group is phenoxy.

As used here, “haloalkoxy” refers to an —O-haloalkyl group. Example haloalkoxy groups include OCF₃, OCF₂CF₃, OCH₂CF₃ and the like.

As used herein, “aralkyl” or “arylalkyl” refers to an alkyl moiety substituted by an aryl group. Example aralkyl groups include benzyl and naphthylmethyl groups. In some embodiments, arylalkyl groups have from 7 to 11 carbon atoms.

As used herein, “carbocyclylalkyl” refers to an alkyl moiety substituted by a carbocyclyl group. Example carbocyclylalkyl groups include “aralkyl” (alkyl substituted by aryl) and “cycloalkylalkyl” (alkyl substituted by cycloalkyl).

As used herein, “heterocyclylalkyl” refers to an alkyl moiety substituted by a heterocarbocyclyl group. Example heterocarbocyclylalkyl groups include “heteroarylalkyl” (alkyl substituted by heteroaryl) and “heterocycloalkylalkyl” (alkyl substituted by heterocycloalkyl).

As used herein, “carbocyclyloxy” refers —O-carbocyclyl.

As used herein, “heterocyclyloxy” refers to —O-heterocyclyl.

As used herein, “amino” refers to an NH₂ group. “Alkylamino” refers to an amino group substituted by an alkyl group and “dialkylamino” refers to an amino group substituted by two alkyl groups.

As used herein, “aminocarbonyl” refers to CONH₂.

As used herein, “alkylaminocarbonyl” refers to CONH(alkyl).

As used herein, “dialkylaminocarbonyl” refers to CON(alkyl)₂.

As used herein, “carboxy” or “carboxyl” refers to COOH.

As used herein, “carboxy alkyl ester” refers to COO-alkyl.

As used herein, “carboxy aryl ester” refers to COO-aryl.

As used herein, “hydroxy” refers to OH.

As used herein, “mercapto” refers to SH.

As used herein, “sulfinyl” refers to SO.

As used herein, “sulfonyl” refers to SO₂.

As used herein, “aminosulfonyl” refers to SO₂NH₂.

As used herein, “alkylaminosulfonyl” refers to SO₂NH(alkyl).

As used herein, “dialkylaminosulfonyl” refers to SO₂N(alkyl)₂.

As used herein, “arylsulonyl” refers to SO₂-aryl.

As used herein, “arylsulfinyl” refers to SO-aryl.

As used herein, “alkylsulfonyl” refers to SO₂-alkyl.

As used herein, “alkylsulfinyl” refers to SO-alkyl.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The neutral forms of the compounds of the present invention may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The compounds of the present invention can possess chiral or asymmetric carbon atoms (optical centers) or double bonds; thus, the racemates, diastereomers, geometric isomers and individual optical isomers are all intended to be encompassed within the scope of the present invention.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

Some of the compounds of the invention can exist in unsolvated forms as well as solvated forms, including hydrated forms; all forms of which are encompassed within the scope of the present invention. Certain compounds of the present invention may exist in multiple crystalline or amorphous forms. In general, all physical forms are equivalent for the uses contemplated by the present invention and are intended to be within the scope of the present invention.

In some embodiments, compounds of the invention include “prodrugs”. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention.

Synethesis

The compounds of the present invention can be prepared in a variety of ways known to one skilled in the art of organic synthesis. The compounds of the present invention can be synthesized using the methods as hereinafter described below, together with synthetic methods known in the art of synthetic organic chemistry or variations thereon as appreciated by those skilled in the art.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatograpy (HPLC) or thin layer chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Green, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of this invention can be prepared using the exemplary reaction pathways and techniques described below.

According to some methods for preparing compounds of Formula I, the hydroxamic acid group can be prepared from the corresponding ester 1 in either one or two steps (Scheme 1). The one-step protocol involves direct conversion of the hydroxamic acid by reaction with a base, such as NaOMe (Kim, D. K., et al. J. Med. Chem. 2003, 46, 5745.) or NaOH (Carpino, L. A.; Xia, J.; El-Faham, A. J. Org. Chem. 2004, 69, 54.), and hydroxylamine in a polar solvent such as methanol.

The two-step protocol involves first alkaline hydrolysis of the corresponding ester 1 (where alkyl is a suitable group such as methyl, ethyl, allyl, benzyl- or t-butyl) using a suitable aqueous base, such as lithium hydroxide, sodium hydroxide, or potassium hydroxide, preferably in a homogeneous aqueous-organic solvent mixture, such as THF/H₂O or MeOH/H₂O. Alternatively, the carboxylic acid can also be prepared by acid hydrolysis of the corresponding ester using a suitable aqueous acid, such as hydrochloric acid in aqueous dioxane, at a suitable temperature. If the ester is a methyl ester, demethylation can be conducted using anhydrous TFA (D. C. Tabor and S. A. Evans, Jr. Synthetic Comm. 1982, 12, 855) or anhydrous AlBr₃ and EtSH (Pal, S. K.; Gupta, P. D.; Mukherjee, D. Tetrahedron 2002, 58, 1765). Other methods recognizable by those skilled in the art as suitable for converting esters to acids can also be employed, such as hydrogenolysis of the benzyl ester using hydrogen and palladium on carbon, acid-promoted cleavage of t-butyl esters under anhydrous conditions, and palladium-catalyzed cleavage of allyl esters.

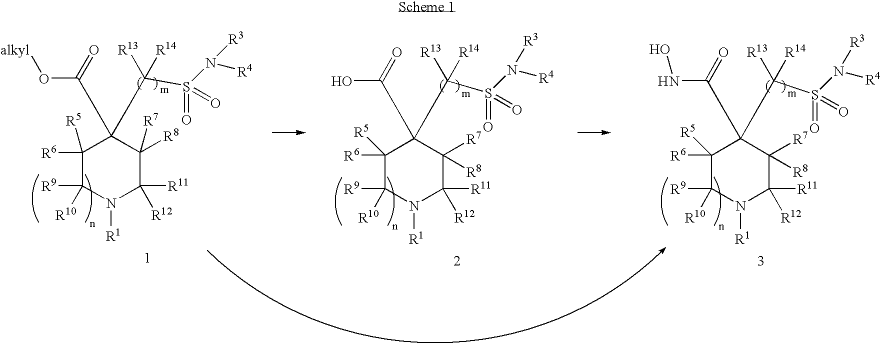

The second step involves the coupling of the carboxylic acid 2 and hydroxylamine, which may be conducted under a variety of reaction conditions known to one skilled in the art of organic synthesis. One illustrative example, utilizes a peptide coupling agent, such as 1,1′-carbonyl-diimidazole, benzyotriazol-1-yloxy-tris(dimethylamino)phosphonium hexafluorophosphate (“PyBOP”), 4-(4,6-dimethoxy[1,3,5]triazin-2-yl)-4-methylmorpholinium chloride (DMTMM), etc. in the presence of a base, such as N,N-diisopropylethylamine, N-methylmorpholine, or triethylamine. In a second example, the carboxylic acid 2 can be converted to the acid chloride by reaction with oxalyl chloride or thionyl chloride followed by reaction with hydroxylamine in the presence of a base. In a third example, the carboxylic acid 2 can be converted to a mixed anhydride by reaction with an alkyl chloroformate in the presence of a base, such as N,N-diisopropyl ethylamine, N-methylmorpholine, or triethylamine, followed by reaction with hydroxylamine (Barraclough, P.; Caldwell, A. G.; Harris, C. J.; Jackson, W. P.; Whittle, B. J. R. J. Chem. Soc., Perkin Trans. 1 1989, 1815).

The coupling reactions described above can also be conducted with oxygen-protected hydroxylamine derivatives (i.e., a suitable protecting group known to those skilled in the art, such as benzyl, t-butyl, t-butyldimethylsilyl, or t-butyldiphenylilyl) and when desired the hydroxylamine can be liberated under the appropriate deprotection reaction conditions known to those skilled in the art. Illustrative examples of converting esters such as 1 to oxygen-protected hydroxamic acid derivatives include using Weinreb's trimethylaluminum reaction conditions (J. I. Levin, E. Turos, S. M. Weinreb, Syn. Comm. 1982, 12, 989) or Roskamp's bis[bis(trimethylsilyl)amido]tin reagent (W. -B. Wang, E. J. Roskamp, J. Org. Chem. 1992, 57, 6101). For further elaboration on appropriate protection/deprotection sequences see T. W. Greene and P. G. M. Wuts Protective Groups in Organic Synthesis 3^{rd} ed. 1999, John Wiley & Sons, NY, USA.

The sulfonamide moiety of compounds of Formula I can be introduced utilizing a variety of different synthetic routes. One approach is to conduct a SN₂ reaction on an iodo-alkyl compound of structure 3 with either potassium thioacetate or sodium hydrogen sulfite to afford 4 or 5, respectively (Scheme 2). It will be obvious to one skilled in the art that the iodine can be substituted by a variety of different leaving groups, such as a different halogen or an activated hydroxyl (i.e. mesylate, tosylate, alkoxyphosphonium ions).

In the case of the thioacetate intermediate 4, oxidation and chlorination can occur in one step with chlorine gas in dichloromethane, water, and acetic acid to form the corresponding sulfonyl chloride 6 (see W. J. Moree, et al J. Org. Chem. 1995, 60, 5157. Scheme 2). Alternative chlorinating or brominating reagents can be employed, such as sulfuryl chloride, bromine, or NBS.

In the case of the sulfonic acid intermediate 5, chlorination can be achieved by reaction with thionyl chloride or phosphorous pentachloride to afford the corresponding sulfonyl chloride 6 (see H. Matter et al. Bioorg. Med. Chem. 1987, 30, 2232. Scheme 2).

The sulfonyl chloride 6 can then be reacted with any primary or secondary amine in the presence of a base, such as N,N-diisopropylethylamine, N-methylmorpholine, or 2,6-lutidine and 4-dimethylaminopyridine (DMAP) to afford the sulfonamide 1 (Scheme 2: LG is I, Br, Cl, O-activated, etc.). In the case where the amine is a primary amine, the resulting secondary amine free NH can be further functionalized or protected as is desired by methods known to one skilled in the art.

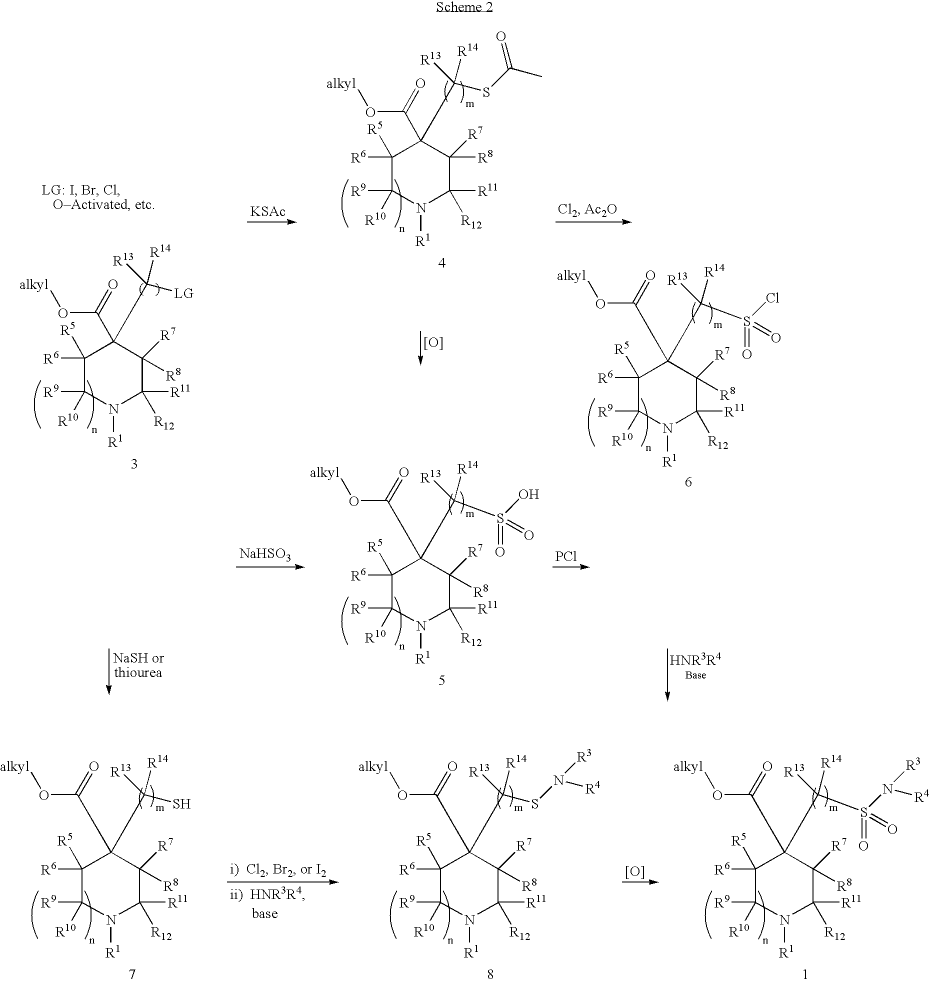

A third approach is to conduct an SN₂ reaction on an iodo-alkyl compound of structure 3 with either sodium thiol or thiourea to afford the corresponding thiol compound 7 (Scheme 2; LG refers to leaving group such as I, Br, Cl, O-activated and the like). Subsequent halogenation followed by reaction with an amine and oxidation affords the sulfonamide 1.

For the case where m=2 in compounds of Formula I, the sulfonamide can be prepared as described above or the corresponding methyl sulfonamide anion can be added to an alkyl group with a leaving group attached 3 (e.g. halogen or an activated hydroxyl (i.e. mesylate, tosylate, alkoxyphosphonium ions), etc.) in a SN₂ manner (Scheme 3; LG refers to leaving group such as I, Br, Cl, O-activated and the like). Alternatively, the corresponding methyl sulfonamide anion can be condensed with a ketone or aldehyde 3′ to afford the corresponding alcohol sulfonamide 1′. It is also feasible to condense the corresponding Wittig sulfonamide with either a ketone or aldehyde 3′ to afford the corresponding alkenyl sulfonamide 1″.

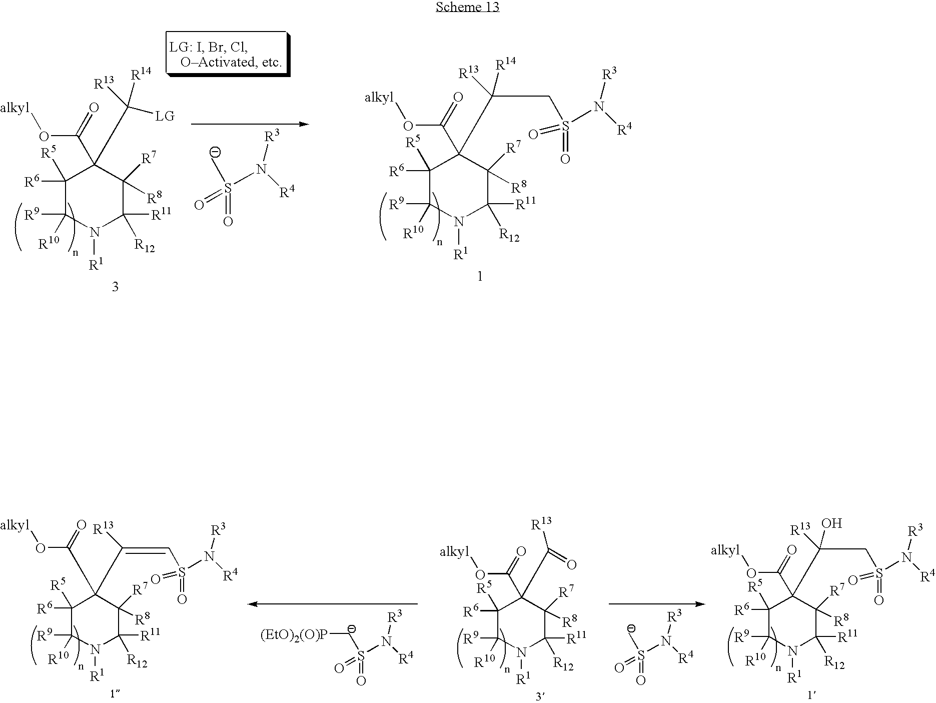

The sulfonamides 1, 1′, and 1″ described above can be further functionalized at R¹³ and R¹⁴ by transformations that would be apparent to those skilled in the art. For example, 1 and 1′ can undergo a second sulfonamide anion formation followed by reaction with an electrophile. The functionalized sulfonamide 1′ can also be subjected to various functional group interconversions known to those skilled in the art, such as oxidation, elimination, alkylation, acylation, substitution, halogenation, etc. Similarly, the functionalized sulfonamide 1″ can also be subjected to various functional group interconversions known to those skilled in the art, such as oxidation, reduction, nucleophilic and electrophilic additions, pericyclic reactions, etc. For further discussion of functional group interconversions see F. A. Carey and R. J. Sundberg, Advanced Organic Chemistry, Part B: Reactions and Synthesis 3^{rd} ed. 1990, Plenum Press, NY, USA and J. March, Advanced Organic Chemistry: Reactions, mechanisms, and structure 4^{th} ed., 1992, John Wiley and Sons, NY, USA.

In the descriptions above in which HNR³R⁴ is an aryl piperazine, piperidine, tetrahydropyridine, or pyrrolidine the aforementioned syntheses can be utilized when the material is not commercially available. The proposed synthetic routes are not intended to be comprehensive and alternative syntheses known by those skilled in the art of organic synthesis may be used.

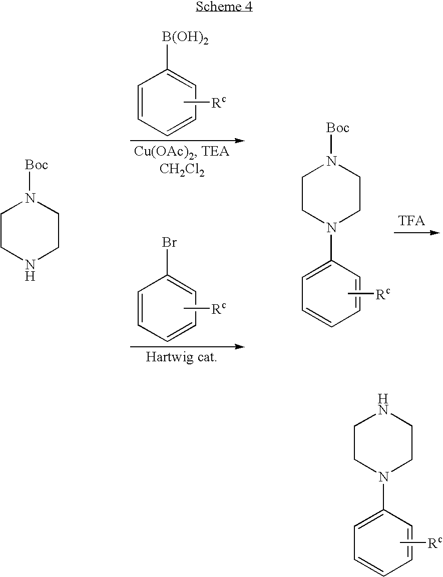

Aryl piperazine intermediates can be prepared by reacting Boc-piperazine with a variety of boronic acids under the catalysis of copper (II) acetate (Combs, A. P.; Tadesse, S.; Rafalski, M.; Haque, T. S.; Lam, P. Y. S. J. Comb. Chem. 2002, 4, 179) or with a variety of aryl halides using Hartwig's catalyst (Louie, J; Hartwig, J. F. Tetrahedron Lett. 1995, 36, 3609) (Scheme 4). After removal of the Boc group with TFA, the secondary amine can be coupled with sulfonyl chloride 6 to furnish compounds of formula 1 as previously described above (Scheme 2). Aryl piperazine intermediates can also be prepared through classical ring closure of appropriately substituted anilines and bis-(2-chloroethyl)amine hydrochloride in the presence of base (E. Mishani, et. al. Tetrahedron Lett. 1996, 37, 319), or through direct nucleophilic aromatic substitution of the piperazine (S. M. Dankwardt, et al., Tetrahedron Lett. 1995, 36, 4923).

Aryl tetrahydropyridines can be prepared by first converting the tert-butoxycarbonylpiperid-4-one to the corresponding enol triflate using LDA and N-phenyltrifluoromethanesulfonamide (Scheme 5; M+ refers to Mg, Li, Na, or other metal cation). The aryl triflate can then be used directly in a Suzuki-type coupling reaction with a variety of arylboronic acids to produce the aryltetrahydropyridines (M. G. Bursavich, D. H. Rich, Org. Lett. 2001, 3, 2625).

Alternatively, the enol triflate can be converted to the corresponding enol boronic ester or acid via palladium mediated coupling and then subsequently coupled with an aryl halide through a Suzuki-type reaction (Scheme 5). After removal of the Boc group with TFA, the secondary amine can be coupled with sulfonyl chloride 6 to furnish compounds of formula 1 as previously described (Scheme 2). It should be noted that aryl tetrahydropyridines can also be prepared through alternative methods known by those skilled in the art of organic synthesis, such as direct nucleophilic addition of an aryl anion to a piperidone followed by dehydration and deprotection of the resultant alcohol compound.

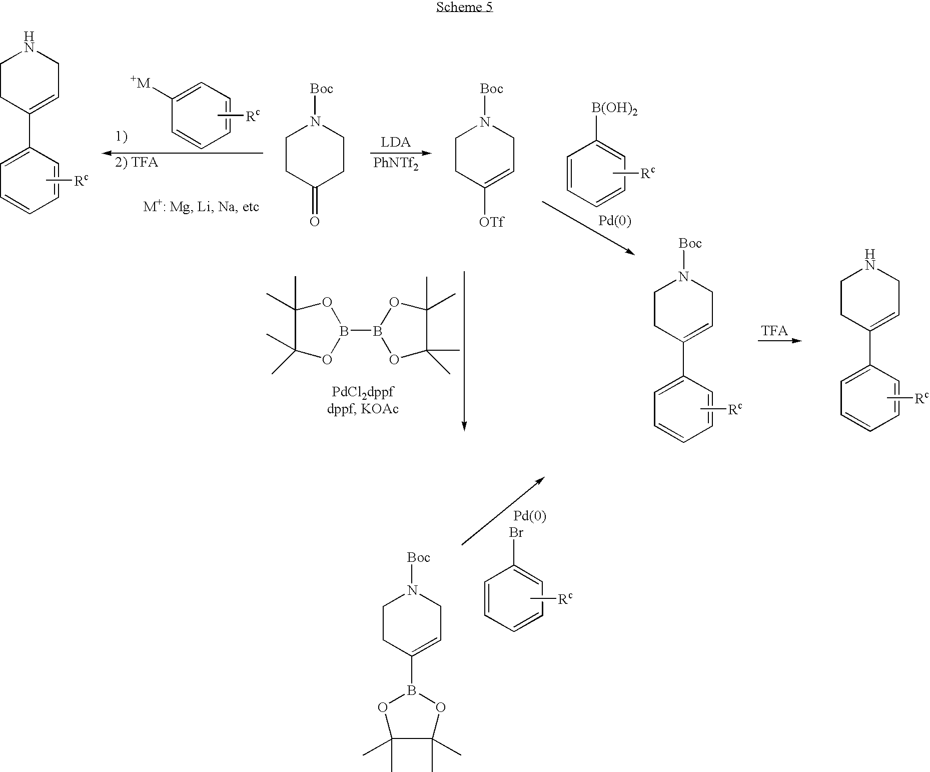

Aryl piperidine derivatives can be prepared by catalytic hydrogenation of the above formed aryltetrahydropyridines or by coupling a 4-bromopyridine with an aryl boronic acid in the presence of a palladium catalyst followed by hydrogenation (Scheme 6).

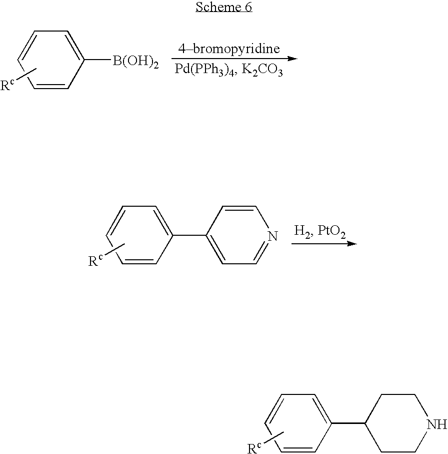

Arylpyrrolidine derivatives can be prepared from optically pure (R)-phenylsuccinic acid by reduction with lithium aluminum hydride to afford the corresponding diol (Scheme 7). Following bis-mesylation the aromatic ring may be substituted by methods known to those skilled in the art of organic synthesis, such as electrophilic aromatic substitution. After the desired derivitization of the aryl ring, the bis-mesylate is reacted with benzylamine in the presence of triethylamine to afford the N-benzylpyrrolidine. At this stage the pyrrolidine amine can then be liberated under hydrogenation conditions. The amine can then be reacted with sulfonyl chloride 6 to afford the sulfonamide 1 as previously described (Scheme 2).

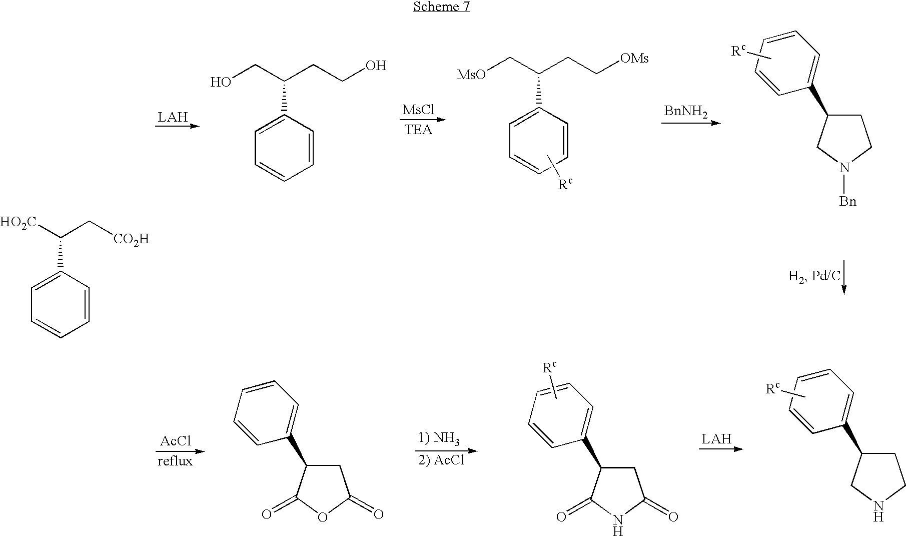

Alternatively, (R)-phenylsuccinic acid can be refluxed in acetyl chloride to afford the corresponding anhydride (Scheme 7). At this stage the aromatic ring may be substituted by methods known to those skilled in the art of organic synthesis, such as electrophilic aromatic substitution. Following the desired derivitization of the aromatic ring, the lactone is treated with ammonia and acetyl chloride under reflux conditions to afford the imide. Reduction with lithium aluminum hydride affords the arylpyrrolidine, which can be coupled with the sulfonyl chloride 6 to furnish compounds of Formula I as previously described (Scheme 2).

Aryl pyrrolidine compounds can also be synthesized through Suzuki-type coupling via an enol triflate or enol boronate intermediates in a similar method as described in Scheme 5. In another example, phenylpyrrolidine derivatives can be prepared through a classic direct nucleophilic addition of an aryl anion to a pyrrolidone followed by dehydration of the resultant alcohol compound and then asymmetric hydrogenation.

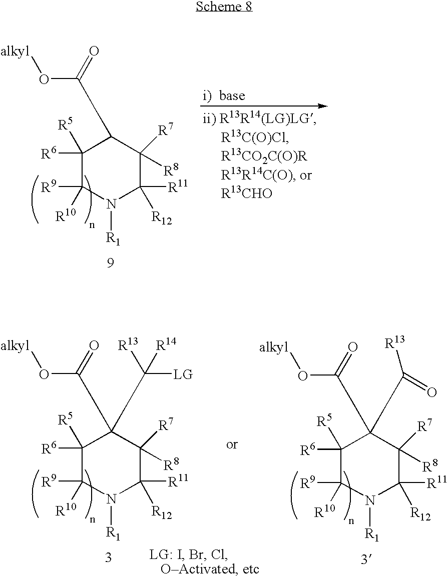

The compounds of formula 3 can be prepared by reaction of the enolate of ester 9 with an electrophile (E⁺), such as an alkyl or acyl halide, anhydride, ketone, aldehyde, etc. as described in Scheme 8 (LG refers to leaving group such as I, Br, Cl, O-activated and the like). The enolate of ester 9 is formed by reaction of ester 9 with a base, such as lithium diisopropylamine (LDA) or sodium hexamethyldisilylamide (NaHMDS), at low temperature (<−10° C.) in an anhydrous solvent, preferably an ethereal solvent (e.g. tetrahydrofuran or diethyl ether).

In the case where the enolate is condensed with an aldehyde or ketone the corresponding alcohol 3 that is formed can be converted to a leaving group (LG) for subsequent displacement either by activation, such as conversion to a mesylate, tosylate, or alkoxyphosphonium ion, or converted to a halide by methods known to one skilled in the art. Alternatively, if the alcohol is a secondary alcohol it can be oxidized to the ketone 3′. The ketone 3′ can then undergo the reactions previously described in Scheme 3.

In the case where the electrophile is an acyl chloride or an anhydride the product formed will be the corresponding ketone 3′. The ketone 3′ can then undergo the reactions previously described in Scheme 3.

The compounds of Formula I wherein R¹ is a carbamate, amide, urea or sulfonamide can be prepared by utilizing the general synthetic route described in Scheme 9 (LG refers to leaving group such as I, Br, Cl, O-activated and the like). It should be recognized by one skilled in the art that in the proceeding illustrative examples sulfonylation and acylation of the amine can be conducted under analogous reaction conditions. Therefore, when the term acylation is used sulfonylation is also implied. Thus compounds of formula 9 can be prepared by reaction of the amine 10 with the corresponding alkyl or aryl chloroformate or anhydride and a base such as N,N-diisopropylethylamine, N-methylmorpholine, or triethylamine. The reaction can also be conducted under biphasic conditions using THF and a mineral base solution, such as 1.0 N NaOH, 1.0 N KOH, etc. Alternatively, the amine 1 can be acylated by reaction with the corresponding carboxylic acid under conventional peptide coupling reaction conditions known by those skilled in the art. Illustrative examples of suitable coupling agents include 1,1′-carbonyl-diimidazole, benzyotriazol-1-yloxy-tris(dimethylamino)phosphonium hexafluorophosphate (“PyBOP”), 4-(4,6-dimethoxy[1,3,5]triazin-2-yl)-4-methylmorpholinium chloride (DMTMM), etc. in the presence of a base, such as N,N-diisopropylethylamine, N-methylmorpholine, or triethylamine.

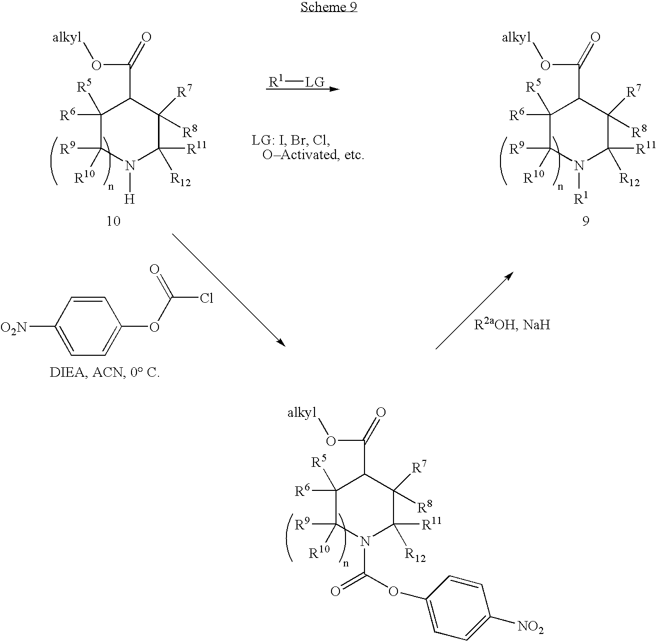

Alternatively if R¹ is a carbamate, the amine 10 can be reacted with p-nitrophenyl chloroformate to afford the corresponding carbamate, followed by reaction with the desired alcohol in the presence of a base, such as NaH, to afford 9.

If R¹ is a cyanoguanidine, the piperidine 10 can be reacted with commercially available diphenyl cyanocarbonimidate in the presence of a base, such as triethylamine, in refluxing acetonitrile. Subsequent reaction with an amine in a refluxing solvent, such as i-propanol or acetonitrile, forms the desired cyanoguanidine. It should be recognized by those skilled in the art that reversing the order of these two steps will also afford the same cyanoguanidine. Alternatively, cyanoguanidine analogs may be prepared from the corresponding isothiocyanate as recently described by Poindexter et al. and Perez-Medrano et al. (Poindexter, G. S. et al. Bioorg. Med. Chem. 2004, 12, 507. and Perez-Medrano, A. et al. Bioorg Med. Chem. Lett. 2004, 14, 397.)

If R¹ is a nitroguanidine, the piperidine 10 can be reacted with commercially available S-methyl N-nitroimidothiocarbamate in the presence of a base, such as NaOH (Scheme 10). Subsequent alkylation of the intermediate with the corresponding alkyl halide in a polar solvent (e.g. DMF) and in the presence of a base, such as potassium carbonate or sodium hydride, affords the nitroguanidine (Maienfisch, P. et al. Tetrahedron Lett. 2000, 41, 7187).

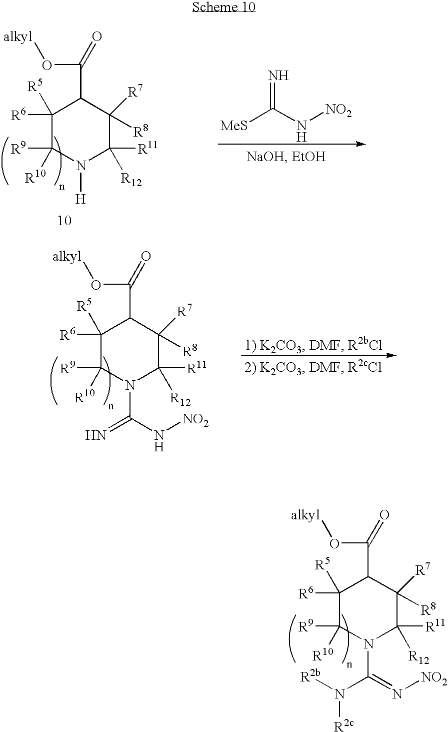

It will be realized to one skilled in the art that the R¹ substituent can be introduced at other stages of the synthesis. For example, the R¹ substituent can be introduced at a later stage, such as after the sulfonamide moiety is in-tact, as exemplified in Scheme 11 (LG refers to leaving group such as I, Br, Cl, O-activated and the like; PG refers to a protecting group such as Bn, Cbz, Boc, COOMe and the like). Thus the nitrogen is protected in the previous steps and is then liberated prior to substitution. For further discussion on appropriate protection/deprtoection sequences see T. W. Greene and P. G. M. Wuts Protective Groups in Organic Synthesis 3^{rd} ed. 1999, John Wiley & Sons, NY, USA.

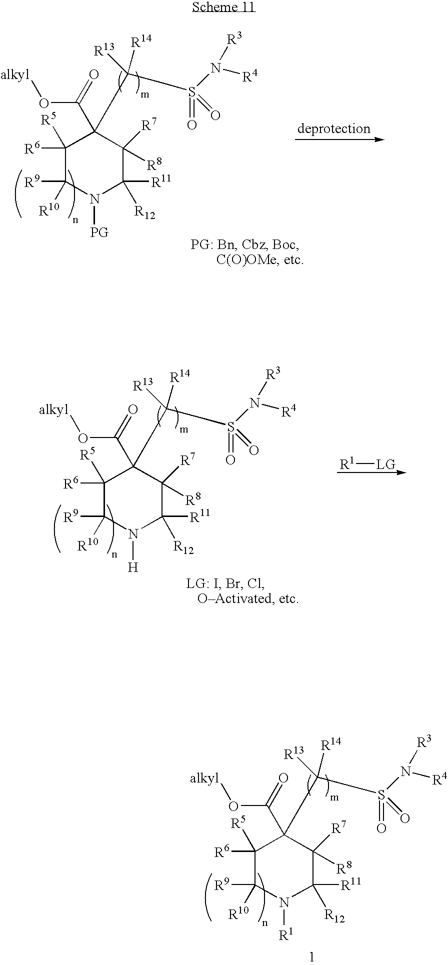

The compounds of formula 10, if not commercially available, can be prepared utilizing a variety of synthetic methods known to those skilled in the art of organic synthesis. For current applicable synthetic methods for the construction of 5, 6, and 7-membered heterocycles see T. Eicher and S. Hauptmann The Chemistry of Heterocycles 2003, John Wiley and Sons, NY, USA; Enders, D. et al. Pure and Applied Chemistry 2001, 73, 573; O'Hagan, D. et al., Nat. Prod. Rep. 2000, 17, 435; and O'Hagan, D. et al., Nat. Prod. Rep. 1197, 14, 637.

The compounds of the invention, where n=0, can be prepared by a variety of synthetic approaches to one skilled in the art of organic synthesis. One synthetic approach for the preparation of pyrrolidine analogs is the reduction of the corresponding pyrrole. The substituted pyrroles can be prepared using a variety of methods known by one skilled in the art, such as the Knorr, Paal-Knorr, and Hantzsch syntheses and variations thereof. Alternatively, the readily available pyrrole 11 can be reduced to afford the 3-substituted pyrrolidine or can be functionalized at the 2 and/or 5 positions by either electrophilic substitution or ortho-lithiation followed by reaction with an electrophile (Scheme 12). Subsequent manipulations could then be performed prior to the reduction of the pyrole to afford the desired substituted pyrrolidine.

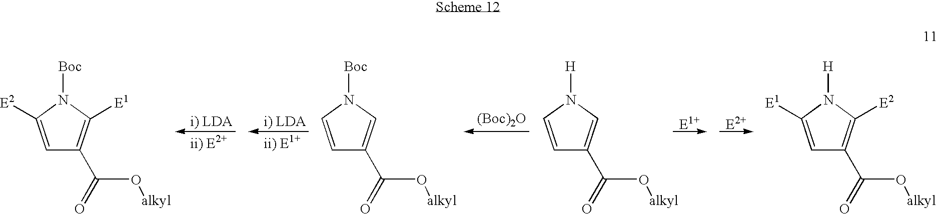

A second synthetic approach involves utilizing readily available starting materials 3-pyrolidinone 12 and lactam 13 and conducting a reduction followed by removal of the nitrogen protecting group to afford the free amine 14 (Scheme 13).

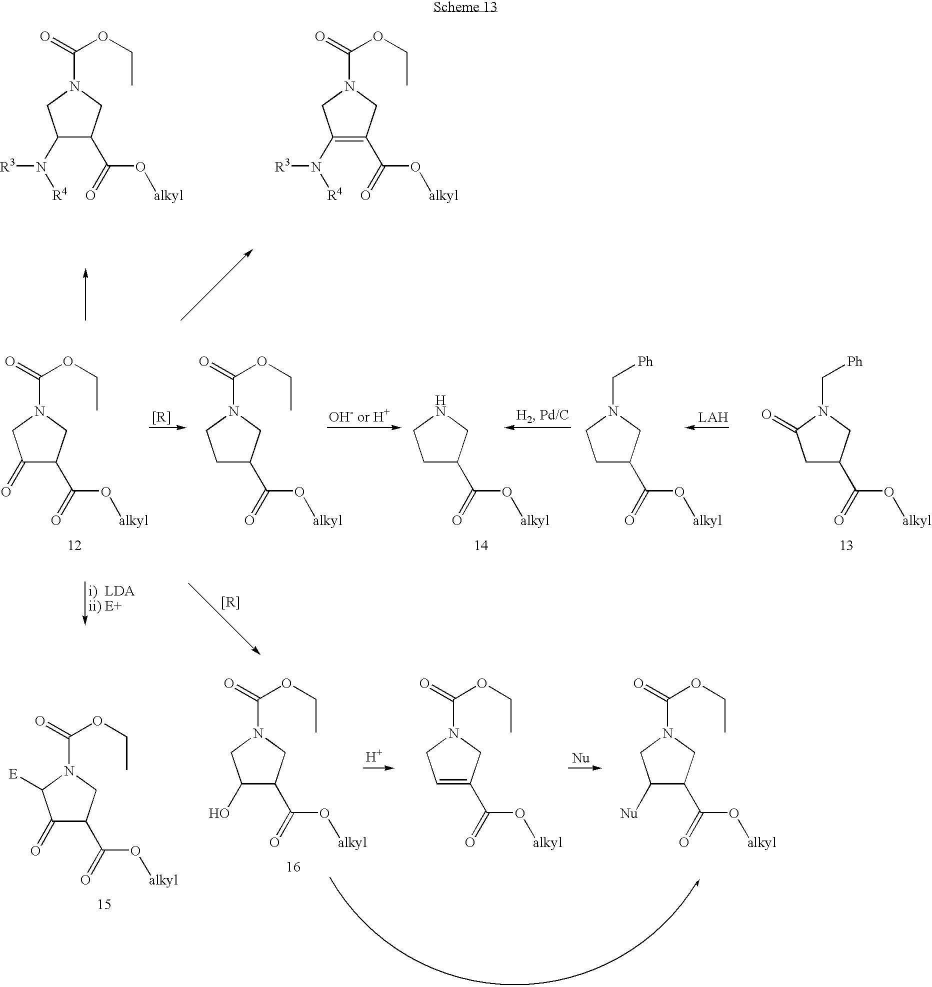

The ketone functionality in 3-pyrrolidinone 12 can be exploited for further derivitization of the pyrrolidine ring through transformations that will be apparent to one skilled in the art (Scheme 13). For example, ketone 12 can undergo a Wittig reaction, epoxidation, Mannich-type reaction to form an enamine, reductive amination (for stereoselective examples see Wenjun, T., et al. J. Amer. Chem. Soc. 2003, 125, 9570 and Lee, H. -S. J. Org. Chem. 2001, 66, 3597.), or reacted with various nucleophiles, such as carbanions (for stereoselective examples see Baldwin, J. E. et al. Tetrahedron 1997, 53, 5233).

In a second example, the ketone can be stereospecifically reduced and then the resultant alcohol can undergo various reactions known by one skilled in the art, such as activation (i.e. converted to a mesylate, tosylate, alkoxyphosphonium ions) followed by nucleophilic displacement, alkylation, or elimination (for reviews on stereoselective reductions see Tramontini, M. Synthesis 1982, 602. and Greeves, N. In Comrehensive Organic Synthesis; Trost, B. M.; Fleming, I., Eds.; Pergamon Press, Oxford, 1991; Vol. 8, pp 1-24. Scheme 13).

In a third example, the β-keto ester 12 can form the corresponding dianion followed by reaction with an electrophile to form the 2-substituted pyrrolidine derivative (Gallagher, T. et al. J. Chem. Soc., Chem. Comm. 1990, 1047 and 1992, 166. Scheme 13). The intermediate can then be further elaborated by methods known to one skilled in the art, such as decarboxylation, reduction, enolate formation, etc.

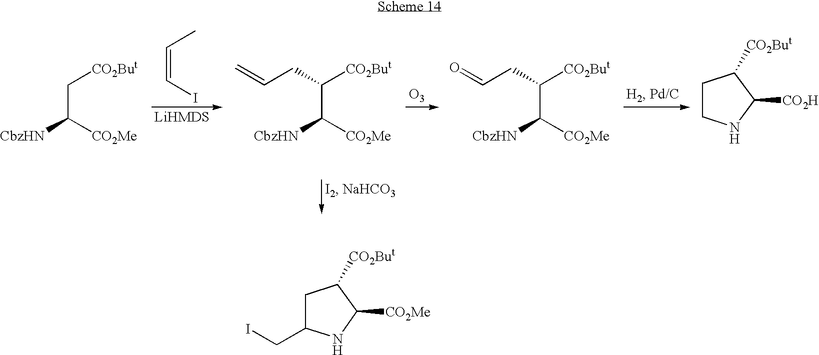

A third synthetic approach is to introduce the desired substituents and desired stereochemistry to an open chain compound and then to perform the cyclization to form the corresponding pyrrolidine ring. One example of this approach is alkylation of a chiral differentiated aspartate diester derivative followed by either ozonolysis and cyclization or iodolactamization (Scheme 14). The intermediates that are formed can then be further functionalized and substituted by methods known to one skilled in the art of organic synthesis.

A second example of this approach involves a dipolar addition to afford the trans differentiated 3,4-diester pyrrolidine (M. Joucla and J. Mortier, Chem. Commun. 1985, 1566, Scheme 15).

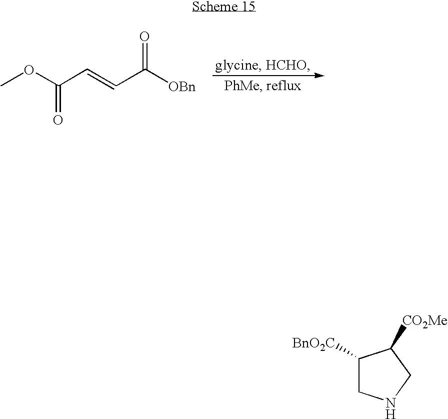

A third example of this approach involves the metal carbenoid N—H insertion of a polyfunctionalized chiral building block 17 as depicted in Scheme 16 (Davis, F. A., Yang, B., Deng, J. J. Org. Chem. 2003, 68, 5147). The resulting β-keto ester 18 can then be further functionalized or substituted prior to the conversion of the ketone 18 to the alkyl ester, by methods known to one skilled in the art (Badham, N. F. et al. Org. Proc. & Res. Dev. 2003, 7, 101), such as homologation of the ketone using a Peterson-type reaction with a 1,3-dithiane followed by conversion to the appropriate ester (Street, L. J., et al. J. Med. Chem. 1990, 33, 2690).

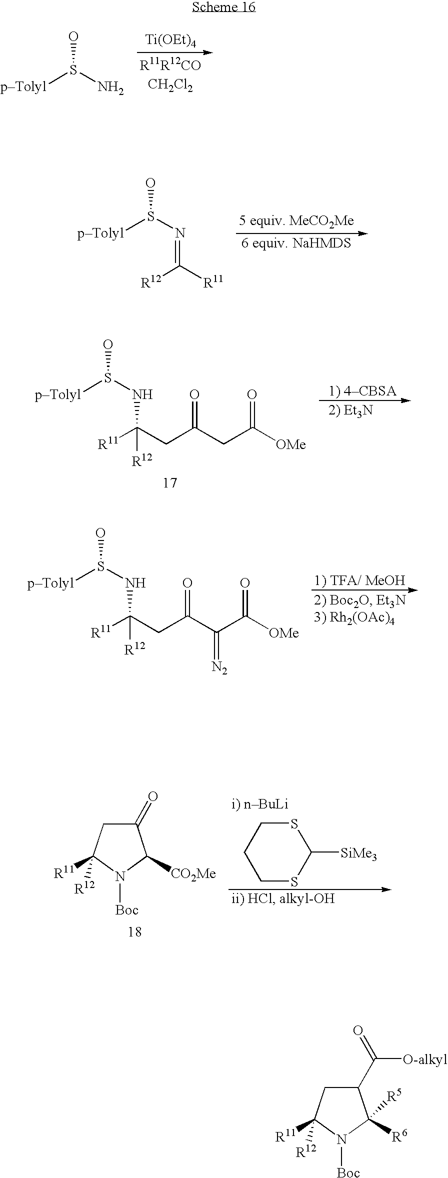

The three synthetic approaches described above can also be applied with minor modifications, that will be obvious to one skilled in the art, for the preparation of piperidines with the general formula of 10, where n=1. For example, reduction of a partially or fully unsaturated pyridine system, derivitization of commercially available functionalized 4-ester piperidines, or cyclization of an appropriately substituted and functionalized open chain compound. Exemplary methods of the latter approach include cyclization of 1-amino-5-haloalkanes, Dieckmann cyclization, Thorpe-Ziegler cyclization, ring-closing metathesis (for a recent review see F. X. Felpin and J. Lebreton Eur. J. Org. Chem. 2003, 3693.), and Mannich reaction of 1-amino-5-aldehydes or ketones. For recent reviews on the synthesis of piperidines and piperidones see S. Laschat and T. Dickner Synthesis 2000, 1781 and Weintraub, P. M. et al. Tetrahedron 2003, 59 2953.

A particularly useful approach is to use the chiral polyfunctionalized chiral building block 17 to prepare various substituted 4-piperidones (Scheme 17, for a review see Davis, F. A.; Chao, B.; Andemichael, Y. W.; Mohanty, P. K.; Fang, T.; Burns, D. M.; Rao, A.; Szewczyk, J. M. Heteroatom Chem. 2002, 13, 1. also see Davis, F. A.; Rao, A.; Carrol, P. J. Org. Lett. 2003, 5, 3855). The 4-piperidones can then be converted to the corresponding 4-ester piperidines by methods known to one skilled in the art. Examples of converting 4-piperidones to 4-ester-piperidines include homologation of the ketone using a Peterson-type reaction with a 1,3-dithiane followed by methanolysis (Street, L. J., et al. J. Med. Chem. 1990, 33, 2690), enolate formation followed by carbonylation (Roche, C. et al. Org. Lett. 2003, 5, 1741), or by other methods known to one skilled in the art (Bosch, J. et al. J. Chem. Soc. Perkin Trans. 1 1986, 1533).

The synthetic routes and reagents outlined in Scheme 17 (PG refers to a protecing group; E⁺ refers to an electrophile; FGI refers to functional group interconversion) may be modified or attenuated to accommodate certain functionalities or substituents in the molecule and the intermediates that are formed may be further derivatized or functionalized. It is implied that the sequential use of LDA and an electrophile can be repeated, if possible, to introduce two substituents. It should also be noted that due to the chirality of the polyfunctionalized chiral building block 17 the reactions outlined in Scheme 17 should occur in a stereoselective manner and the resulting diastereomeric mixtures may be separated by conventional methods known to one skilled in the art of organic synthesis to afford optically pure compounds. For further discussion on stereochemistry and separation of diastereoisomers consult E. L Eliel and S. H Wilen Stereochemistry of Organic Compounds, John Wiley & Sons, 1994, NY, USA.

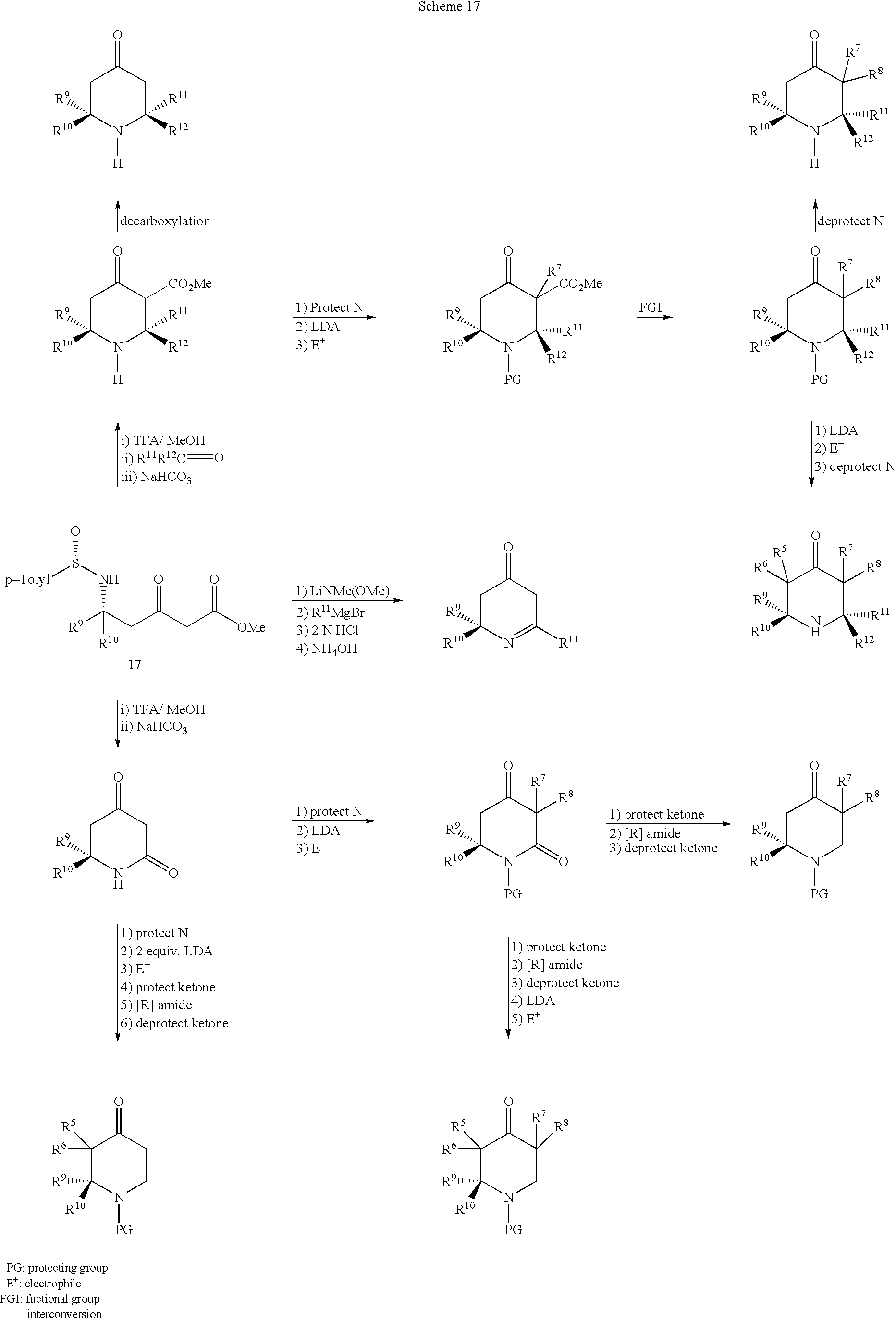

The synthetic approaches that were used above for the construction of pyrrolidines and piperidines can also be applied for the construction of azepine derivatives of formula 10, where n=2, with minor modifications, that will be obvious to one skilled in the art. For example, reduction of a partially or fully unsaturated azepine system, derivitization and functionalization of commercially available ethyl 1-boc-5-oxo-hexahydro-1H-azepine-4-carboxylate, cyclization of an appropriately substituted and functionalized open chain compound, such as ring-closing metathesis or ring expansion.

Particularly useful is the ring expansion of appropriately substituted piperidines, which can be substituted and functionalized by the methods that were previously described. For recent advances on ring expansion of piperidines to afford azepane and azepine derivatives consult Chong, H -S. et al. J. Chem. Soc., Perkin Trans. 1 2002, 2080; T. Morie and K. Shiro Heterocycles 1998, 43, 427; Adams, P. C. al. J. Chem. Soc., Perkin Trans. 1 1995, 2355; DeRuiter, J. et al. J. Heter. Chem. 1992, 29, 779; and Dowd, P. et al. Tetrahedron 1991, 47, 4847.

Fused bicyclic ring systems of Formula II can be prepared by the methods that were disclosed above as well as by conventional methods known to one skilled in the art of organic synthesis, such as reductive cyclization, Schmidt reaction, Friedlander synthesis, Pfitzinger synthesis, Combes synthesis, Knorr synthesis, Konrad-Limpach synthesis, Skraup and Doebner-Miller synthesis, Meth-Cohn synthesis, Reissert synthesis, Batcho-Leimgruber synthesis, Madelung synthesis, Bischler synthesis, Nenitzescu synthesis, and the Fisher synthesis and variations thereof (T. Eicher and S. Hauptmann The Chemistry of Heterocycles 2003, John Wiley and Sons, NY, USA).

Methods

Compounds of the invention can modulate activity of a metalloprotease. The term “modulate” is meant to refer to an ability to increase or decrease activity of a metalloprotease. Accordingly, compounds of the invention can be used in methods of modulating one or more metalloproteases by contacting the metalloprotease with any one or more of the compounds or compositions described herein. In some embodiments, compounds of the present invention are inhibitors or antagonists of one or more metalloproteases. In further embodiments, the compounds of the invention can be used to modulate a metalloprotease in an individual in need of metalloprotease modulation by administering a modulating amount of a compound of Formula I or II.

Metalloproteases having activity modulated by the compounds of the present invention include any metalloprotease. In some embodiments, the metalloprotease is an ADAM such as, for example, ADAM10, ADAM15, ADAM17 and the like. In some embodiments, the metalloprotease is a matrix metalloprotease such as, for example, MMP12, MMP14, MMP3, MMP2, or MMP9. In some embodiments, the compounds of the invention can inhibit more than one metalloprotease. In some embodiments, the compounds of the invention selectively inhibit one type of metalloprotease over another type of metalloprotease. For example, the compounds of the invention can selectively inhibit members of the ADAM family over MMPs, meaning, for example, that the compounds of the invention are better inhibitors of at least one ADAM than of any MMP. In some embodiments, the compounds show inhibitory activity for an ADAM that is at least about 2-fold, at least about 3-fold, at least about 5-fold, at least about 10-fold, at least about 50-fold or at least about 100-fold greater than for any MMP. In further embodiments, the compounds of the invention are selective for ADAM10, ADAM15, or ADAM17 (TACE) over other members of the ADAM family. For example, the compounds of the invention can have inhibitory activity with respect to ADAM10, ADAM15, or ADAM17 that is at least about 2-fold, at least about 3-fold, at least about 5-fold, at least about 10-fold, at least about 50-fold or at least about 100-fold greater than for other ADAMs. In further embodiments, the compounds of the invention can be selective for MMPs such as MMP2, MMP3, MMP12, MMP 14, MMP9 or selective for other metalloproteases.

The compounds the invention can be used for the treatment of diseases or pathological changes associated with unwanted, abnormal or elevated metalloprotease activity by administering a therapeutically effective amount of a compound of Formula I or II to a patient suffering or likely to suffer from the metalloprotease-associated disease. The disease can be associated with the activity of any one or more metalloproteases such as an ADAM (e.g., ADAM10, ADAM15, ADAM17, etc.) or MMP (e.g., MMP12, MMP14, MMP3, MMP2, or MMP9).

The compounds of the invention can further be used for treating diseases associated with activity of a Her-2 (p185) sheddase, growth factor sheddases, or cytokine sheddases by administering to a patient suffering or likely to from the disease a therapeutically effective amount of one or more compounds of Formula I or II. In some embodiments, the disease is associated with activity of a Her-2 sheddase that cleaves Her-2 to form a membrane-bound p95 “stub” and shed extracellular domain.

Non-limiting examples of diseases associated with metalloprotease activity include Some examples where inhibition of metalloprotease activity would be of benefit include: a) osteoarthritis, b) rheumatic diseases and conditions such as autoimmune disease, rheumatoid arthritis, c) septic arthritis, d) cancer including tumor growth, tumor metastasis and angiogenesis, e) periodontal diseases, f) corneal, epidermal or gastric ulceration (ulcerative conditions can result in the cornea as the result of alkali burns or as a result of infection by Pseudomonas aeruginosa, Acanthamoeba, Herpes simplex and vaccinia viruses), g) proteinuria, h) various cardiovascular and pulmonary diseases such as atherosclerosis, thrombotic events, atheroma, hemodynamic shock, unstable angina, restenosis, heart failure, i) aneurysmal diseases including those of the aorta, heart or brain, j) birth control, k) dystrophobic epidermolysis bullosa, l) degenerative cartilage loss following traumatic joint injury, m) osteopenias and other diseases of abnormal bone loss including osteoporosis, n) tempero mandibular joint disease, o) pulmonary diseases such as chronic obstructive pulmonary disease, p) demyelinating diseases of the nervous system such as multiple sclerosis, q) metabolic diseases including diabetes (with enhanced collagen degradation) and obesity mediated by insulin resistance, macular degeneration and diabetic retinopathy mediated by angiogenesis, cachexia, premature skin aging, r) impaired wound healing including burns, s) decubital ulcers, t) acute and chronic neurodegenerative disorders including stroke, spinal cord and traumatic brain injury, amyotrophic lateral sclerosis, cerebral amyloid angiopathy, CNS injuries in AIDS, Parkinson's disease, Alzheimer's disease, Huntington's diseases, prion diseases, myasthenia gravis, and Duchenne's muscular dystrophy, u) pain, v) autoimmune encephalomyelitis and w) diseases linked to TNFα production and/or signaling such as a wide variety of inflammatory and/or immunomodulatory diseases, including acute rheumatic fever, rheumatoid arthritis, multiple sclerosis, allergy, psoriasis, periodontal diseases, hepatitis, bone resorption, sepsis, gram negative sepsis, septic shock, endotoxic shock, toxic shock syndrome, systemic inflammatory response syndrome, inflammatory bowel diseases including Crohn's disease and ulcerative colitis, Jarisch-Herxheimer reactions, asthma, adult respiratory distress syndrome, acute pulmonary fibrotic diseases, pulmonary sarcoidosis, allergic respiratory diseases, silicosis, coal worker's pneumoconiosis, alveolar injury, hepatic failure, liver disease during acute inflammation, severe alcoholic hepatitis, malaria including Plasmodium falciparum malaria and cerebral malaria, congestive heart failure, damage following heart disease, arteriosclerosis including atherosclerosis, Alzheimer's disease, acute encephalitis, brain injury, pancreatitis including systemic complications in acute pancreatitis, impaired wound healing and immune responses in infection inflammation and cancer, myelodysplastic syndromes, systemic lupus erythematosus, biliary cirrhosis, non-insulin dependent diabetes mellitus, bowel necrosis, psoriasis, cachexia and anorexia, radiation injury, and toxicity following administration of monoclonal antibodies such as OKT3, host-versus-graft reactions including ischemia reperfusion injury and allograft rejections including those of the kidney, liver, heart, and skin, lung allograft rejection including chronic lung allograft rejection (obliterative bronchitis), as well as complications due to total hip replacement, infectious diseases including Mycobacterial infection, meningitis, Helicobacter pylori infection during peptic ulcer disease, Chaga's disease resulting from Trypanosoma cruzi infection, effects of Shiga-like toxin resulting from E. coli infection, the effects of enterotoxin A resulting from Staphylococcus infection, meningococcal infection, and infections from Borrelia burgdorferi, Treponema pallidum, cytomegalovirus, influenza virus, Sendai virus, Theiler's encephalomyelitis virus, and the human immunodeficiency virus (HIV).

In some embodiments, the disease associated with metalloprotease activity is arthritis, cancer, cardiovascular disorders, skin disorders, inflammation or allergic conditions. In further embodiments, the disease is cancer including, for example, breast cancer, ovarian cancer, prostate cancer, colon cancer, pancreatic cancer, gastric cancer, non-small cell lung cancer, glioma and the like.

As used herein, the term “contacting” refers to the bringing together of indicated moieties in an in vitro system or an in vivo system. For example, “contacting” a metalloprotease with a compound of the invention includes the administration of a compound of the present invention to an individual or patient, such as a human, having a metalloprotease, as well as, for example, introducing a compound of the invention into a sample containing a cellular or purified preparation containing the metalloprotease.

As used herein, the terms “individual,” “patient,” and “mammalian subject,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting further development of the pathology and/or symptomatology); and
(3) ameliorating the disease; for example, ameliorating a disease, condition or disorder in an individual that is experiencing or displaying the pathology or symptomatology of the disease, condition or disorder (i.e., reversing the pathology and/or symptomatology).
   Pharmaceutical Formulations and Dosage Forms

When employed as pharmaceuticals, the compounds of Formula I or II can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of Formula I or II above in combination with one or more pharmaceutically acceptable carriers (excipients). In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgement of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral adminstration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

Labeled Compounds and Assay Methods

Another aspect of the present invention relates to radio-labeled compounds of Formula I or II that would be useful not only in radio-imaging but also in assays, both in vitro and in vivo, for localizing and quantitating a metalloprotease in tissue samples, including human, and for identifying metalloprotease ligands by inhibition binding of a radio-labeled compound. Accordingly, the present invention includes metalloprotease assays that contain such radio-labeled compounds.

The present invention further includes isotopically-labeled compounds of Formula I or II. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro metalloprotease labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled ” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for incorporating radio-isotopes into organic compounds are applicable to compounds of the invention and are well known in the art.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to a metalloprotease. Accordingly, the ability of a test compound to compete with the radio-labeled compound for binding to the metalloprotease directly correlates to its binding affinity.

Kits

The present invention also includes pharmaceutical kits useful, for example, in the treatment or prevention of metalloprotease-associated diseases or disorders, such as cancer, which include one or more containers containing a pharmaceutical composition comprising a therapeutically effective amount of a compound of Formula I or II. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

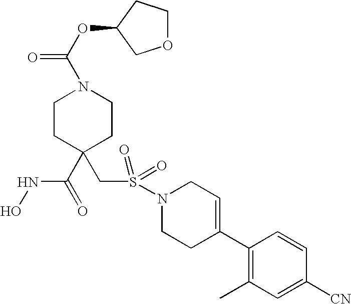

### Example 1

### 4-[4-(4-Cyano-2-methyl-phenyl)-3,6-dihydro-2H-pyridine-1-sulfonylmethyl]-4-hydroxycarbamoyl-piperidine-1-carboxylic acid-3(S)-tetrahydrofuran-3-yl ester

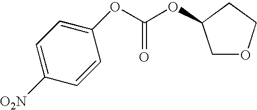 Step A
(S)-3-THF 4-nitrophenyl carbonate

To a stirred solution of p-nitrophenyl chloroformate (6.0 g, 0.029 mol) in anhydrous methylene chloride (60 mL, 1 mol) at 0° C. were added (S)-(+)-3-hydroxytetrahydrofuran (2.44 mL, 0.0302 mol) and 4-methylmorpholine (4.8 mL, 0.043 mol). The reaction mixture was stirred at rt for 4 h. The reaction mixture was quenched with water (50 mL) and extracted with dichloromethane (2×), and the combined organic layers were washed with brine, dried over MgSO₄, filtered and concentrated in-vacuo. The residue was purified by Combiflash with 20-40% EtOAc/Hex. 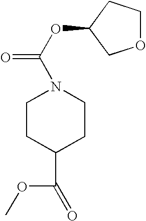
Step B
4-Methyl 1-[(3S)-tetrahydrofuran-3-yl]piperidine-1,4-dicarboxylate

To a solution of (S)-3-THF 4-nitrophenyl carbonate (0.20 g, 0.00079 mol) in dimethyl sulfoxide (4.0 mL, 0.056 mol) were added methyl piperidine-4-carboxylate (0.12 mL, 0.00087 mol) and N,N-diisopropylethylamine (0.28 mL, 0.0016 mol) and the reaction mixture was stirred at 90° C. for 2 h. The mixture was cooled to rt, diluted with ethyl acetate, washed with water, 1 N NaOH (2×), water, 1N HCl (1×), water and brine successively, dried and concentrated. The product was purified by CombiFlash using hexane/EtOAc (max. EtOAc 30%). LC-MS: 258.1 (M+H)⁺. 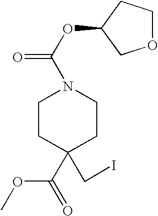
Step C
4-Iodomethyl-piperidine-1,4-dicarboxylic acid 4-methyl ester 1-[(3S)-(tetrahydro-furan-3-yl) ester.

To a solution of lithium diisopropylamide in tetrahydrofuran (1.8 M, 8.8 mL) in THF (30 mL) was slowly added a solution of 4-methyl 1-[(3S)-tetrahydrofuran-3-yl]piperidine-1,4-dicarboxylate (3.7 g, 0.014 mol) in THF (10 mL) at −60° C. and stirred for 1 h. Diiodomethane (1.4 mL, 0.017 mol) was slowly added to the above solution at −60° C. and the mixture was stirred at room temperature for 4 h. The reaction mixture was quenched with saturated NH₄Cl and extracted with ethyl acetate. The combined organic phase was washed with diluted HCl and water successively, dried over sodium sulfate, filtered and concentrated. The crude product was used in the next step without purification. LC-MS: 398.0 (M+H)⁺ 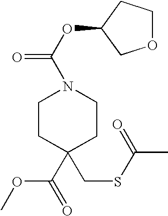
Step D
4-Methyl 1-[(3S)-tetrahydrofuran-3-yl] 4-[(acetylthio)methyl]piperidine-1,4-di-carboxylate.

To a solution of 4-iodomethyl-piperidine-1,4-dicarboxylic acid 4-methyl ester 1-[(3S)-(tetrahydro-furan-3-yl)ester (5.70 g, 0.0144 mol) in N,N-dimethylformamide (40 mL, 0.5 mol) was added potassium thioacetate (2.4 g, 0.022 mol) at room temperature and the resulting mixture was stirred overnight. The mixture was diluted with water and extracted with ethyl acetate. The organic extract was washed with water and brine successively, dried over sodium sulfate, filtered and concentrated. The product was purified by CombiFlash using hexane/EtOAc (max. EtOAc 40%). LC-MS: 346.1 (M+H)⁺ 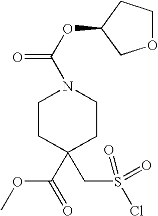
Step E
4-Methyl 1-[(3S)-tetrahydrofuran-3-yl] 4-[(chlorosulfonyl)methyl]piperidine-1,4-dicarboxylate.

To a stirred mixture of 4-methyl 1-[(3S)-tetrahydrofuran-3-yl] 4-[(acetyl-thio)methyl]piperidine-1,4-dicarboxylate (1.90 g, 0.00550 mol), water (100 mL, 4 mol) and acetic acid (5.0 mL, 0.088 mol) was bubbled chlorine gas at 0° C. for 30 min. After stirring for an additional 30 min., the mixture was diluted with methylene chloride, washed with water and brine successively, dried and concentrated to give the crude product, which was puried by CombiFlash using CH₂Cl₂/EtOAc (max. EtOAc 30%). LC-MS: 370.2/372.0 (M+H)⁺ 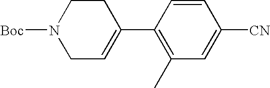
Step F
tert-Butyl 4-(4-cyano-2-methylphenyl)-3,6-dihydropyridine-1(2H)-carboxylate.

A mixture of tert-butyl 4-(4,4,5,5-tetramethyl-1,3,2-dioxaborolan-2-yl)-3,6-dihydropyridine-1(2H)-carboxylate (0.40 g, 0.0013 mol), 4-bromo-3-methylbenzonitrile (0.30 g, 0.0016 mol), [1,1′-bis(diphenylphosphino)ferrocene]dichloropalladium(II), complex with dichloromethane (1:1, 0.063 g, 0.000078 mol) and potassium carbonate (0.54 g, 0.0039 mol) in N,N-dimethylformamide (6.0 mL, 0.077 mol) was stirred under nitrogen at 80° C. overnight. The mixture was diluted with ethyl acetate, washed with water and brine successively, dried and concentrated. The product was purified by CombiFlash using hexane/EtoAc (max. EA 30%) LC-MS: 299.2 (M+H)⁺ 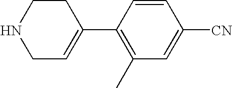
Step G
3-Methyl-4-(1,2,3,6-tetrahydropyridin-4-yl)benzonitrile

A solution of hydrogen chloride in 1,4-dioxane (4.0 M, 4.0 mL) was added to a solution of tert-butyl 4-(4-cyano-2-methyl-phenyl)-3,6-dihydropyridine-1(2H)-carboxylate (0.30 g, 0.0010 mol) in ethyl acetate (1.0 mL, 0.010 mol) at room temperature and the mixture was stirred for 2 h. Diethyl ether was added to the above mixture, and the precipitate was filtered, washed with ether, and dried to provide the desired product as a HCl salt. LC-MS: 199.1 (M+H)⁺. 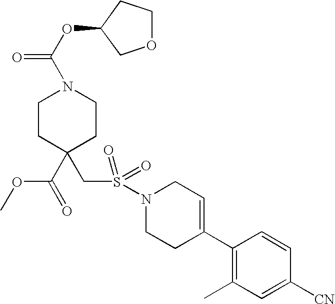
Step H
4-Methyl 1-[(3S)-tetrahydrofuran-3-yl] 4-([4-(4-cyano-2-methylphenyl)-3,6-dihydro-pyridin-1(2H)-yl]sulfonylmethyl)piperidine-1,4-dicarboxylate

To a solution of 3-methyl-4-(1,2,3,6-tetrahydropyridin-4-yl)benzonitrile (0.017 g, 0.000074 mol) and N,N-diisopropylethylamine (0.035 mL, 0.00020 mol) in methylene chloride (1.0 mL) was added 4-methyl 1-[(3S)-tetrahydrofuran-3-yl] 4-[(chlorosulfonyl)methyl]piperidine-1,4-dicarboxylate (0.025 g, 0.000068 mol) in methylene chloride (0.5 mL) at 0° C., and the mixture was stirred at this temperature for 1 h and then at room temperature for 0.5 h. The mixture was diluted with ethyl acetate, washed with water and brine successively, dried over sodium sulfate, filtered and concentrated. The product was purified by CombiFlash using CH₂Cl₂/EtOAc (max. EtOAc 40%) to provide the final product. LC-MS: 532.2 (M+H)⁺ 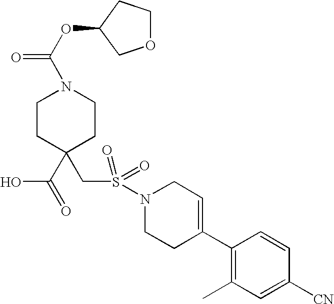
Step I
4-([4-(4-Cyano-2-methylphenyl)-3,6-dihydropyridin-1(2H)-yl]sulfonylmethyl)-1-[(3S)-tetrahydrofuran-3-yloxy]carbonylpiperidine-4-carboxylic acid

To a solution of 4-methyl 1-[(3S)-tetrahydrofuran-3-yl] 4-([4-(4-cyano-2-methylphenyl)-3,6-dihydro-pyridin-1(2H)-yl]sulfonylmethyl)piperidine-1,4-dicarboxylate (0.030 g, 0.000057 mol) in tetrahydrofuran (1.0 mL, 0.012 mol) and water (0.2 mL, 0.01 mol) was added lithium hydroxide, monohydrate (0.0240 g, 0.000573 mol), and the mixture was stirred at room temperature until HPLC data indicated the complete conversion. The mixture was diluted with water and adjusted to be acidic (pH=3) by adding 1N HCl, then extracted with ethyl acetate. The organic phase was washed with brine, dried and concentrated to afford the desired product. LC-MS: 518.2 (M+H)⁺ 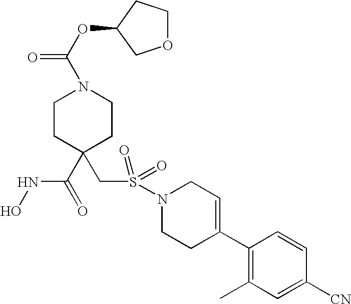
Step J
4-[4-(4-Cyano-2-methyl-phenyl)-3,6-dihydro-2H-pyridine-1-sulfonylmethyl]-4-hydroxycarbamoyl-piperidine-1-carboxylic acid-3(S)-tetrahydrofuran-3-yl ester

To a solution of 4-([4-(4-cyano-2-methylphenyl)-3,6-dihydropyridin-1(2H)-yl]sulfonyl-methyl)-1-[(3S)-tetrahydrofuran-3-yloxy]carbonylpiperidine-4-carboxylic acid (0.028 g, 0.000054 mol) in DMF (0.5 mL) was added benzotriazol-1-yloxytris(dimethylamino)-phosphonium hexafluorophosphate (0.0332 g, 0.0000751 mol) in DMF (0.3 mL) at 0° C. After stirring for 2 min., hydroxylamine hydrochloride (0.011 g, 0.00016 mol) was added to the above solution followed by 4-methylmorpholine (0.0295 mL, 0.000268 mol) at 0° C. The reaction mixture then was warmed to room temperature and stirred until HPLC data indicated that the reaction was complete. The product was purified by prep-HPLC. LC-MS: 533.20 (M+H)⁺

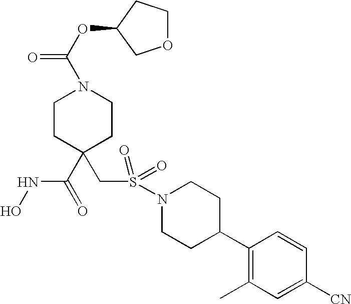

### Example 2

### 4-[4-(4-Cyano-2-methyl-phenyl)-piperidine-1-sulfonylmethyl]-4-hydroxycarbamoyl-piperidine-1-carboxylic acid-3(S)-tetrahydrofuran-3-yl ester

Into a vial were added (3S)-tetrahydrofuran-3-yl 4-([4-(4-cyano-2-methylphenyl)-3,6-dihydropyridin-1(2H)-yl]-sulfonylmethyl)-4-[(hydroxyamino)carbonyl]piperidine-1-carboxylate (3.6 mg, see Example 1) and methanol (2.0 mL). To this solution, palladium (5 wt % on barium sulfate, reduced, Aldrich# 27,299-1) was added under an atmosphere of nitrogen. The reaction mixture then was purged with H₂(g) and the mixture was stirred under hydrogen (balloon) for 1.5 hours. The reaction mixture was filtered through Celite and washed with methanol. The filtrate was concentrated to give the desired product. LC-MS: 535.2 (M+H)⁺

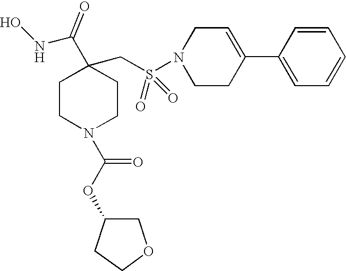

### Example 3

### 4-Hydroxycarbamoyl-4-(4-phenyl-3,6-dihydro-2H-pyridine-1-sulfonylmethyl)piperidine-1-carboxylic acid-3(S)-tetrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-phenyl-1,2,3,6-tetrahydropyridine. LC-MS: 494.2 (M+H)⁺

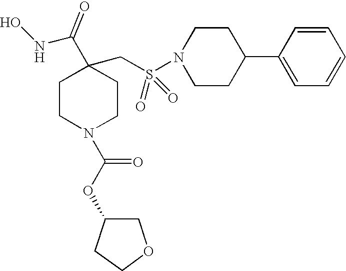

### Example 4

### 4-Hydroxycarbamoyl-4-(4-phenyl-piperidine-1-sulfonylmethyl)-piperidine-1-carboxylic acid-3(S)-tetrahydrofuran-3-yl ester

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-phenyl-1,2,3,6-tetrahydropyridine. LC-MS: 496.2 (M+H)⁺

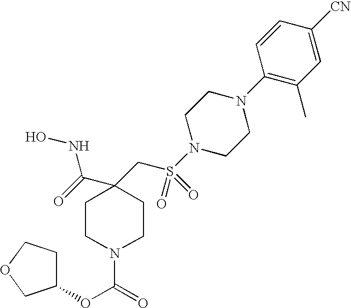

### Example 5

### (3S)-Tetrahydrofuran-3-yl 4-({[4-(4-cyano-2-methylphenyl)piperazin-1-yl]sulfonyl}methyl)-4-[(hydroxyamino)carbonyl]piperidine-1-carboxylate

The title compound was prepared by using a procedure that was analogous to that described for the synthesis of Example 1, with the exception that 4-methyl 1-[(3S)-tetrahydrofuran-3-yl] 4-[(chlorosulfonyl)methyl]piperidine-1,4-dicarboxylate and 3-methyl-4-piperazin-1-ylbenzonitrile, which was prepared by using the procedure described below, were used. LC-MS: 536.20 (M+H)⁺ 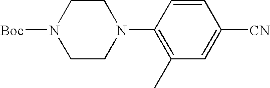
Step A
tert-Butyl 4-(4-cyano-2-methylphenyl)piperazine-1-carboxylate

To a mixture of 4-bromo-3-methylbenzonitrile (1.5 g, 0.0074 mol), 1,1′-bis(diphenylphosphino)ferrocene (200 mg, 0.00037 mol), [1,1′-bis(diphenylphosphino)ferrocene]dichloropalladium-(II), complex with dichloromethane (1:1) (300 mg, 0.00037 mol) and sodium tert-butoxide (820 mg, 0.00854 mol) in tetrahydrofuran (20.0 mL, 0.246 mol) was added tert-butyl piperazine-1-carboxylate (1.5 g, 0.0082 mol) under nitrogen. The reaction mixture then was under reflux for 3 h. After cooling to room temperature, the mixture was diluted with ethyl acetate, washed with diluted HCl, water and brine successively, dried and concentrated. The product was purified by CombiFlash using hexane/EtOAc (max. EA 30%). 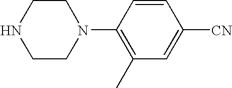
Step B
3-Methyl-4-piperazin-1-ylbenzonitrile

A solution of hydrogen chloride in 1,4-dioxane (4.0 M, 8.0 mL) was added to a solution of tert-butyl 4-(4-cyano-2-methylphenyl)piperazine-1-carboxylate (1.4 g, 0.0046 mol) in ethyl acetate (4.0 mL, 0.041 mol) at room temperature and the mixture was stirred for 2 h. HPLC indicated that the reaction was complete. Diethyl ether was added to the above mixture, and the precipitate was filtered, washed with ether, and dried to provide the desired product as an HCl salt. LC-MS: 202.1 (M+H)⁺

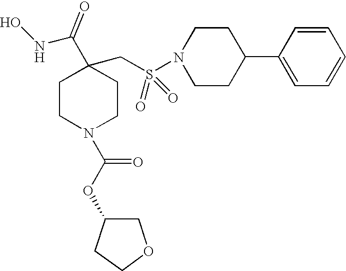

### Example 6

### 4-Hydroxycarbamoyl-4-(4-phenyl-piperazine-1-sulfonylmethyl)-piperidine-1-carboxylic acid-3(S)-tetrahydrofuran-3-yl ester

This compound was prepared substantially as described in Example 5 except starting from 4-methyl 1-[(3S)-tetrahydrofuran-3-yl] 4-[(chlorosulfonyl)methyl]piperidine-1,4-dicarboxylate and phenylpiperazine. LC-MS: 497.2 (M+H)⁺

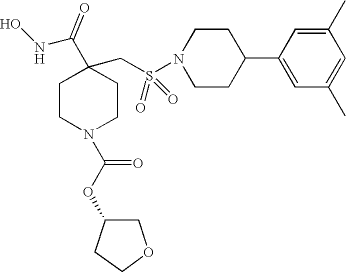

### Example 7

### 4-[4-(3,5-Dimethyl-phenyl)-piperazine-1-sulfonylmethyl]-4-hydroxycarbamoyl-piperidine-1-carboxylic acid-3(S)-tetrahydrofuran-3-yl ester

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 1-(3,5-dimethylphenyl)piperazine. LC-MS: 525.2 (M+H)⁺

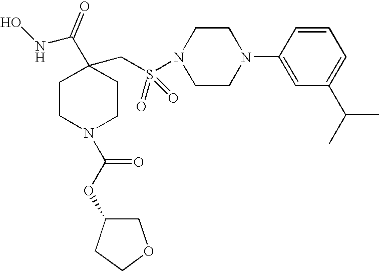

### Example 8

### 4-Hydroxycarbamoyl-4-[4-(3-isopropyl-phenyl)-piperazine-1-sulfonylmethyl]piperidine-1-carboxylic acid-3(S)-tetrahydrofuran-3-yl ester

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 1-(3-i-propylphenyl)piperazine. LC-MS: 539.3 (M+H)⁺

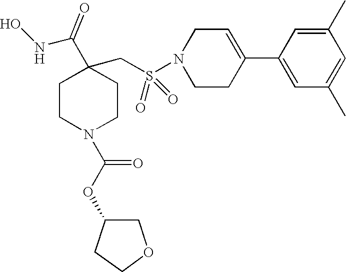

### Example 9

### 4-[4-(3,5-Dimethyl-phenyl)-3,6-dihydro-2H-pyridine-1-sulfonylmethyl]-4-hydroxycarbamoyl-piperidine-1-carboxylic acid-3(S)-tetrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(3,5-dimethylphenyl)-1,2,3,6-tetrahydropyridine. LC-MS: 522.2 (M+H)⁺

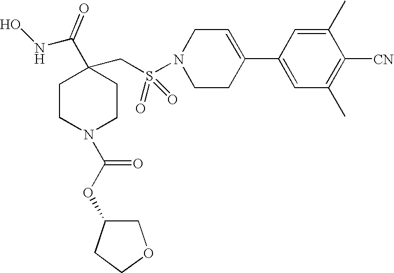

### Example 10

### 4-[4-(4-Cyano-3,5-dimethyl-phenyl)-3,6-dihydro-2H-pyridine-1-sulfonylmethyl]-4-hydroxycarbamoyl-piperidine-1-carboxylic acid-3(S)-etrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 2,6-dimethyl-4-(1,2,3,6-tetrahydropyridin-4-yl)benzonitrile. LC-MS: 547.2 (M+H)⁺

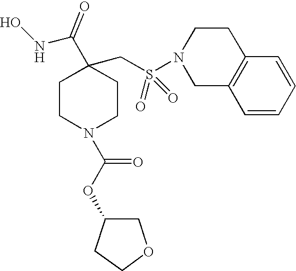

### Example 11

### 4-(3,4-Dihydro-1H-isoquinoline-2-sulfonylmethyl)-4-hydroxycarbamoyl-piperidine-1-carboxylic acid-3(S)-tetrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 1,2,3,4-tetrahydroisoquinoline. LC-MS: 468.2 (M+H)⁺

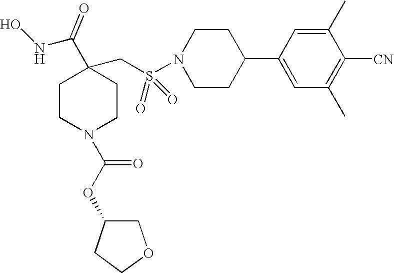

### Example 12

### 4-[4-(4-Cyano-3,5-dimethyl-phenyl)-piperidine-1-sulfonylmethyl]-4-hydroxycarbamoyl-piperidine-1-carboxylic acid-3(S)-tetrahydrofuran-3-yl ester

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 2,6-dimethyl-4-(1,2,3,6-tetrahydropyridin-4-yl)benzonitrile. LC-MS: 549.3 (M+H)⁺

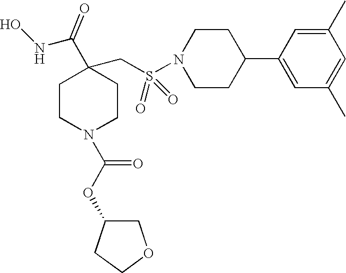

### Example 13

### 4-[4-(3,5-Dimethyl-phenyl)-piperidine-1-sulfonylmethyl]-4-hydroxycarbamoyl-piperidine-1-carboxylic acid-3(S)-tetrahydrofuran-3-yl ester

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(3,5-dimethylphenyl)-1,2,3,6-tetrahydropyridine. LC-MS: 524.2 (M+H)⁺

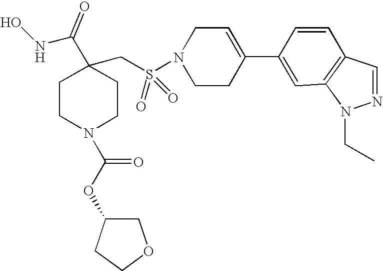

### Example 14

### 4-[4-(1-Ethyl-1H-indazol-6-yl)-3,6-dihydro-2H-pyridine-1-sulfonylmethyl]-4-hydroxycarbamoyl-piperidine-1-carboxylic acid-3(S)-tetrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 1-ethyl-6-(1,2,3,6-tetrahydropyridin-4-yl)-1H-indazole (for the preparation, see Bartsch R. A.; Yang, W., J. Heterocyclic Chem., 21, 1063 (1984) and Schumann, P.; Collot, V.; Hommet, Y.; et al., Bioorg. Med. Chem. Lett., 11, 1153 (2001)). LC-MS: 562.1 (M+H)⁺

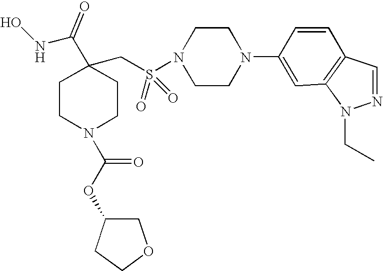

### Example 15

### 4-[4-(1-Ethyl-1H-indazol-6-yl)-piperazine-1-sulfonylmethyl]-4-hydroxycarbamoyl-piperidine-1-carboxylic acid-3(S)-tetrahydrofuran-3-yl ester

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 1-ethyl-6-(piperazin-4-yl)-1H-indazole (for the preparation see Bartsch R. A.; Yang, W., J. Heterocyclic Chem., 21, 1063 (1984) and Schumann, P.; Collot, V.; Hommet, Y.; et al., Bioorg. Med. Chem. Lett., 11, 1153 (2001))n. LC-MS: 565.1 (M+H)⁺

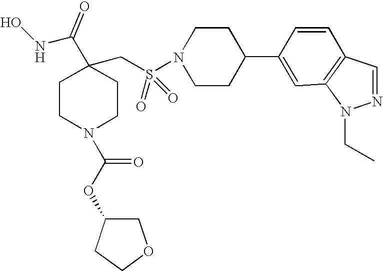

### Example 16

### 4-[4-(1-Ethyl-1H-indazol-6-yl)-piperidine-1-sulfonylmethyl]-4-hydroxycarbamoyl-piperidine-1-carboxylic acid-3(S)-tetrahydrofuran-3-yl ester

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 1-ethyl-6-(1,2,3,6-tetrahydropyridin-4-yl)-1H-indazole (for the preparation see Bartsch R. A.; Yang, W., J. Heterocyclic Chem., 21, 1063 (1984) and Schumann, P.; Collot, V.; Hommet, Y.; et al., Bioorg. Med. Chem. Lett., 11, 1153 (2001)). LC-MS: 564.2 (M+H)⁺

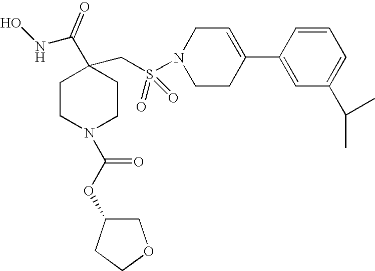

### Example 17

### 4-Hydroxycarbamoyl-4-[4-(3-isopropyl-phenyl)-3,6-dihydro-2H-pyridine-1-sulfonylmethyl]-piperidine-1-carboxylic acid-3(S)-tetrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(3-i-propylphenyl)-1,2,3,6-tetrahydropyridine. LC-MS: 536.2 (M+H)⁺

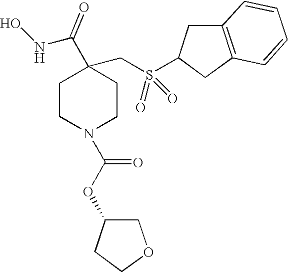

### Example 18

### 4-(1,3-Dihydro-isoindole-2-sulfonylmethyl)-4-hydroxycarbamoyl-piperidine-1-carboxylic acid-3(S)-tetrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and isoindoline. LC-MS: 454.2 (M+H)⁺

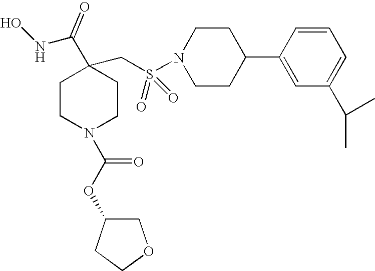

### Example 19

### 4-Hydroxycarbamoyl-4-[4-(3-isopropyl-phenyl)-piperidine-1-sulfonylmethyl]piperidine-1-carboxylic acid-3(S)-tetrahydrofuran-3-yl ester

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(3-i-propylphenyl)-1,2,3,6-tetrahydropyridine. LC-MS: 538.3 (M+H)⁺

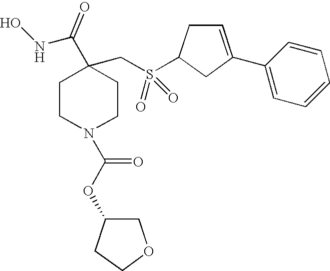

### Example 20

### 4-Hydroxycarbamoyl-4-(3-phenyl-2,5-dihydro-pyrrole-1-sulfonylmethyl)-piperidine-1-carboxylic acid-3(S)-tetrahydrofuran-3-yl ester

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 3-phenyl-2,5-dihydro-1H-pyrrole. LC-MS: 480.2 (M+H)⁺

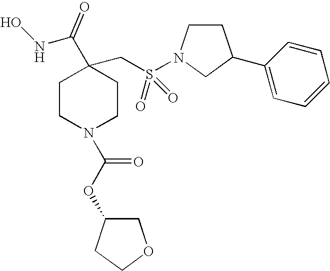

### Example 21

### 4-Hydroxycarbamoyl-4-(3-phenyl-pyrrolidine-1-sulfonylmethyl)-piperidine-1-carboxylic acid-3(S)-tetrahydrofuran-3-yl ester

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 3-phenyl-2,5-dihydro-1H-pyrrole. LC-MS: 482.2 (M+H)⁺

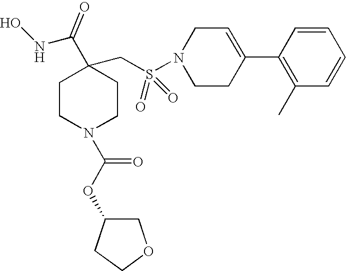

### Example 22

### (3S)-Tetrahydrofuran-3-yl 4-[(hydroxyamino)carbonyl]-4-({[4-(2-methylphenyl)-3,6-dihydropyridin-1(2H)-yl]sulfonyl}methyl)piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(2-methylphenyl)-1,2,3,6-tetrahydropyridine. LC-MS: 508.2 (M+H)⁺

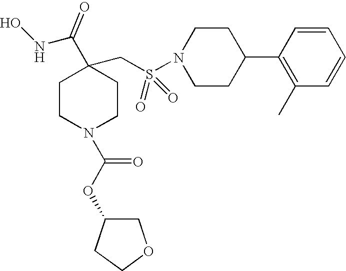

### Example 23

### (3S)-Tetrahydrofuran-3-yl 4-[(hydroxyamino)carbonyl]-4-({[4-(2-methylphenyl)piperidin-1-yl]sulfonyl}methyl)piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(2-methylphenyl)-1,2,3,6-tetrahydropyridine. LC-MS: 510.2 (M+H)⁺

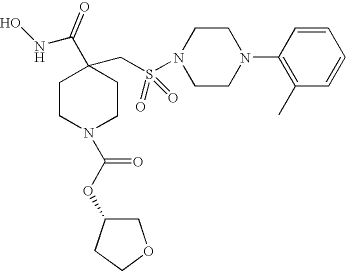

### Example 24

### (3S)-Tetrahydrofuran-3-yl 4-[(hydroxyamino)carbonyl]-4-({[4-(2-methylphenyl)piperazin-1-yl]sulfonyl}methyl)piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(2-methylphenyl)piperazine. LC-MS: 511.2 (M+H)⁺

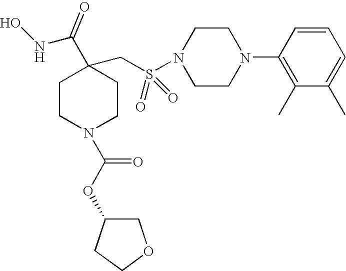

### Example 25

### (3S)-Tetrahydrofuran-3-yl 4-({[4-(2,3-dimethylphenyl)piperazin-1-yl]sulfonyl}methyl)-4-[(hydroxyamino)carbonyl]piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(2,3-dimethylphenyl)piperazine. LC-MS: 501.1 (M+H)⁺

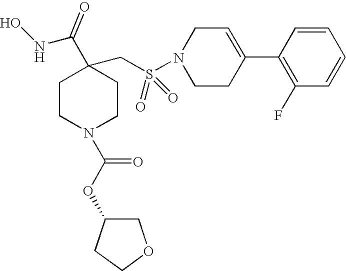

### Example 26

### (3S)-Tetrahydrofuran-3-yl 4-({[4-(2-fluorophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(2-fluorophenyl)-1,2,3,6-tetrahydropyridine. LC-MS: 512.1 (M+H)⁺

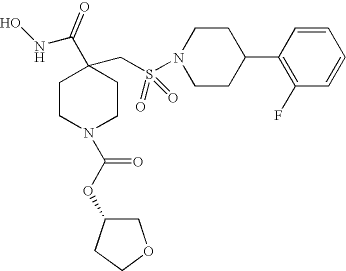

### Example 27

### (3S)-Tetrahydrofuran-3-yl 4-({[4-(2-fluorophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(2-fluorophenyl)-1,2,3,6-tetrahydropyridine LC-MS: 514.6 (M+H)⁺

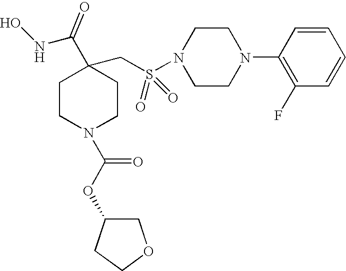

### Example 28

### (3S)-Tetrahydrofuran-3-yl 4-({[4-(2-fluorophenyl)piperazin-1-yl]sulfonyl}methyl)-4-[(hydroxyamino)carbonyl]piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(2-fluorophenyl)piperazine. LC-MS: 515.6 (M+H)⁺

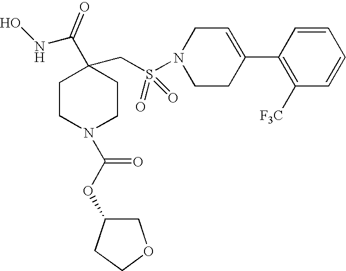

### Example 29

### (3S)-Tetrahydrofuran-3-yl 4-[(hydroxyamino)carbonyl]-4-({[4-[2-(trifluoromethyl)phenyl]-3,6-dihydropyridin-1(2H)-yl]sulfonyl}methyl)piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(2-trifluoromethylphenyl)-1,2,3,6-tetrahydropyridine. LC-MS: 562.2 (M+H)⁺

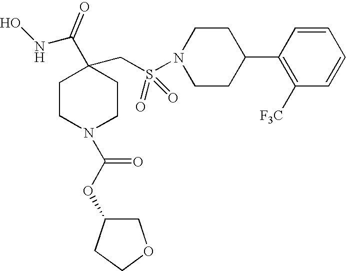

### Example 30

### (3S)-Tetrahydrofuran-3-yl 4-[(hydroxyamino)carbonyl]-4-[({4-[2-(trifluoromethyl)phenyl]piperidin-1-yl}sulfonyl)methyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(2-trifluoromethylphenyl)-1,2,3,6-tetrahydropyridine. LC-MS: 564.6 (M+H)⁺

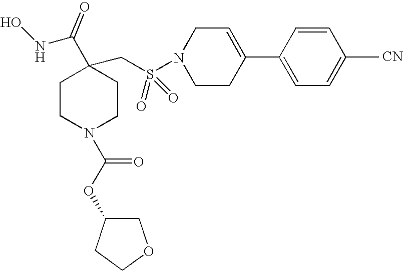

### Example 31

### (3S)-Tetrahydrofuran-3-yl 4-({[4-(4-cyanophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(1,2,3,6-tetrahydropyridin-4-yl)benzonitrile. LC-MS: 519.2 (M+H)⁺

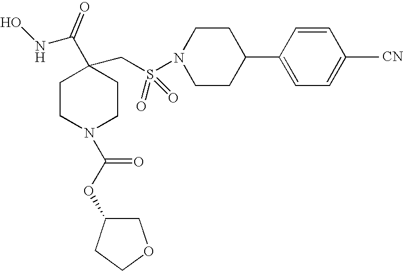

### Example 32

### (3S)-Tetrahydrofuran-3-yl 4-({[4-(4-cyanophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(1,2,3,6-tetrahydropyridin-4-yl)benzonitrile. LC-MS: 521.6 (M+H)⁺

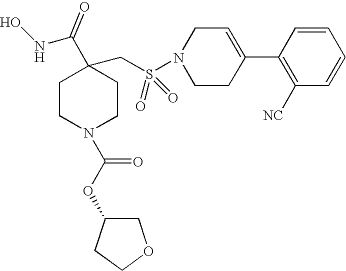

### Example 33

### (3S)-Tetrahydrofuran-3-yl 4-({[4-(2-cyanophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 2-(1,2,3,6-tetrahydropyridin-4-yl)benzonitrile. LC-MS: 519.2 (M+H)⁺

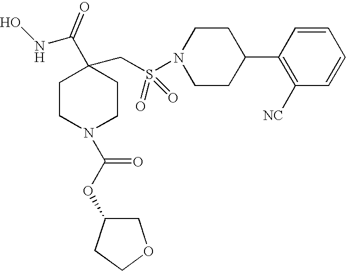

### Example 34

### (3S)-Tetrahydrofuran-3-yl 4-({[4-(2-cyanophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 2-(1,2,3,6-tetrahydropyridin-4-yl)benzonitrile. LC-MS: 521.6 (M+H)⁺

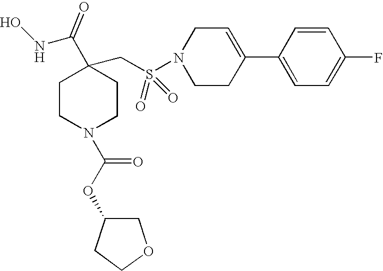

### Example 35

### (3S)-Tetrahydrofuran-3-yl 4-({[4-(4-fluorophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(4-fluorophenyl)-1,2,3,6-tetrahydropyridine. LC-MS: 512.6 (M+H)⁺

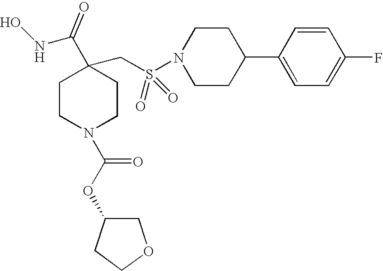

### Example 36

### (3S)-Tetrahydrofuran-3-yl 4-({[4-(4-fluorophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(4-fluorophenyl)-1,2,3,6-tetrahydropyridine. LC-MS: 514.6 (M+H)⁺

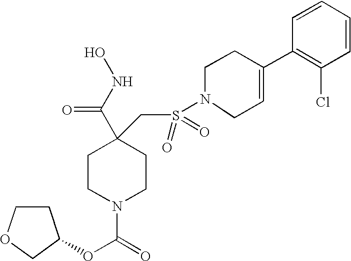

### Example 37

### (3S)-Tetrahydrofuran-3-yl 4-({[4-(2-chlorophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(2-chlorophenyl)-1,2,3,6-tetrahydropyridine. LC-MS: 528.1/530.1 (M+H)⁺

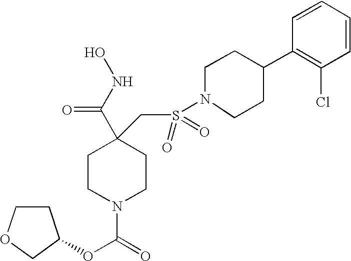

### Example 38

### (3S)-Tetrahydrofuran-3-yl 4-({[4-(2-chlorophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(2-chlorophenyl)-1,2,3,6-tetrahydropyridine. LC-MS: 530.1/532.1 (M+H)⁺

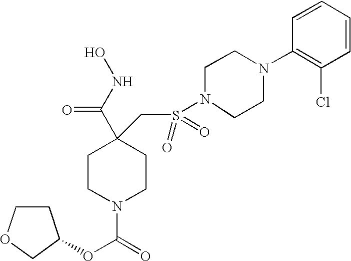

### Example 39

### (3S)-Tetrahydrofuran-3-yl 4-({[4-(2-chlorophenyl)piperazin-1-yl]sulfonyl}methyl)-4-[(hydroxyamino)carbonyl]piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(2-chlorophenyl)piperazine. LC-MS: 531.1/533.1 (M+H)⁺

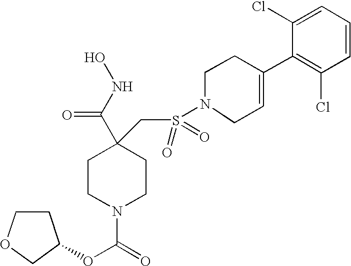

### Example 40

### (3S)-Tetrahydrofuran-3-yl 4-({[4-(2,6-dichlorophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(2,6-dichlorophenyl)-1,2,3,6-tetrahydropyridine. LC-MS: 562.1/564.1 (M+H)⁺

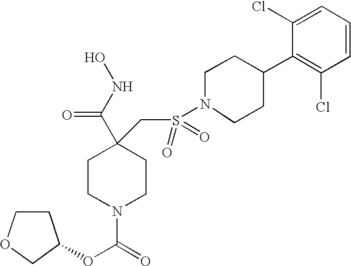

### Example 41

### (3S)-Tetrahydrofuran-3-yl 4-({[4-(2,6-dichlorophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(2,6-dichlorophenyl)-1,2,3,6-tetrahydropyridine LC-MS: 564.1/566.1 (M+H)⁺

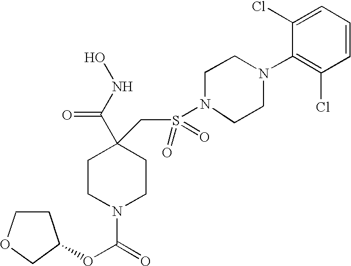

### Example 42

### (3S)-Tetrahydrofuran-3-yl 4-({[4-(2,6-dichlorophenyl)piperazin-1-yl]sulfonyl}methyl)-4-[(hydroxyamino)carbonyl]piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(2,6-dichlorophenyl)piperazine. LC-MS: 565.1/567.1 (M+H)⁺

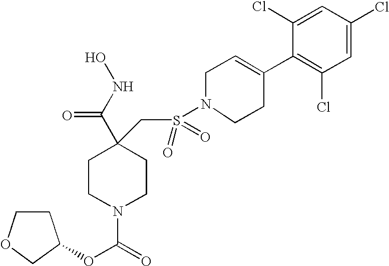

### Example 43

### (3S)-Tetrahydrofuran-3-yl 4-({[4-(2,4,6-trichlorophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(2,4,6-trichlorophenyl)-1,2,3,6-tetrahydropyridine. LC-MS: 598.1/596.1/600.1 (M+H)⁺

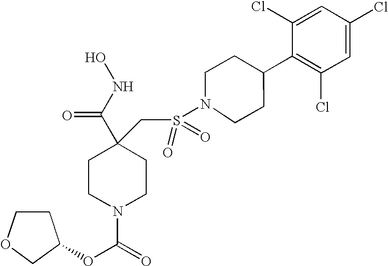

### Example 44

### (3S)-Tetrahydrofuran-3-yl 4-({[4-(2,4,6-trichlorophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(2,4,6-trichlorophenyl)-1,2,3,6-tetrahydropyridine. LC-MS: 600.1/598.1/602.1 (M+H)⁺

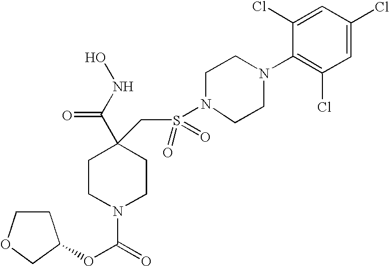

### Example 45

### (3S)-Tetrahydrofuran-3-yl 4-({[4-(2,4,6-trichlorophenyl)piperazin-1-yl]sulfonyl}methyl)-4-[(hydroxyamino)carbonyl]piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(2,4,6-trichlorophenyl)piperazine. LC-MS: 599.1/597.1/601.1 (M+H)⁺

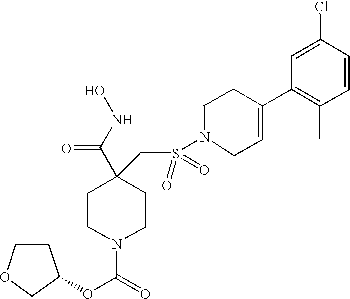

### Example 46

### (3S)-Tetrahydrofuran-3-yl 4-({[4-(3-chloro-6-methylphenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(5-chloro-2-methylphenyl)-1,2,3,6-tetrahydropyridine. LC-MS: 542.1/544.1 (M+H)⁺

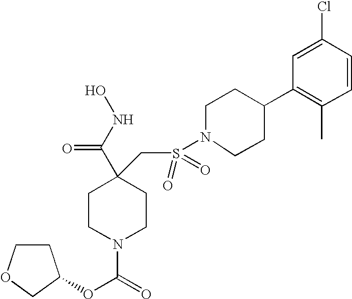

### Example 47

### (3S)-Tetrahydrofuran-3-yl 4-({[4-(3-chloro-6-methylphenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(5-chloro-2-methylphenyl)-1,2,3,6-tetrahydropyridine. LC-MS: 544.1/546.1 (M+H)⁺

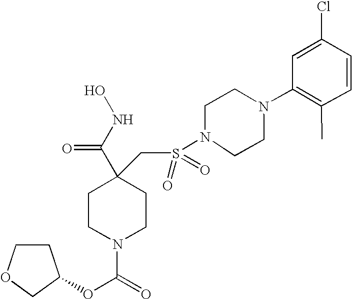

### Example 48

### (3S)-Tetrahydrofuran-3-yl 4-({[4-(3-chloro-6-methylphenyl)piperazin-1-yl]sulfonyl}methyl)-4-[(hydroxyamino)carbonyl]piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(5-chloro-2-methylphenyl)piperazine. LC-MS: 545.1/547.1 (M+H)⁺

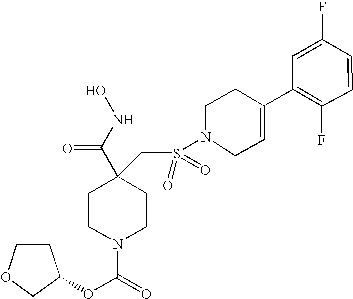

### Example 49

### (3S)-Tetrahydrofuran-3-yl 4-({[4-(2,5-difluorophenyl)-3,6-dihydropyridin-1(2H)-yl]sulfonyl}methyl)-4-[(hydroxyamino)carbonyl]piperidine-1-carboxylate

This compound was prepared substantially as described in Example 1 except starting from 4-methyl 1-[(3S)-tetrahydrofuran-3-yl] 4-[(chlorosulfonyl)methyl]piperidine-1,4-dicarboxylate and 4-(2,5-difluorophenyl)-1,2,3,6-tetrahydropyridine. LC-MS: 530.2 (M+H)⁺

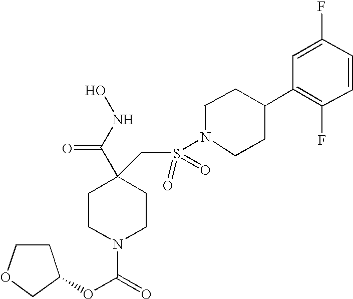

### Example 50

### (3S)-Tetrahydrofuran-3-yl 4-({[4-(2,5-difluorophenyl)piperidin-1-yl]sulfonyl}methyl)-4-[(hydroxyamino)carbonyl]piperidine-1-carboxylate

This compound was prepared substantially as described in Example 2 except starting from 4-methyl 1-[(3S)-tetrahydrofuran-3-yl] 4-[(chlorosulfonyl)methyl]piperidine-1,4-dicarboxylate and 4-(2,5-difluorophenyl)-1,2,3,6-tetrahydropyridine. LC-MS: 531.2 (M+H)⁺

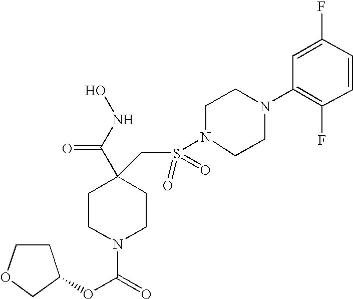

### Example 51

### (3S)-Tetrahydrofuran-3-yl 4-({[4-(2,5-difluorophenyl)piperazin-1-yl]sulfonyl}methyl)-4-[(hydroxyamino)carbonyl]piperidine-1-carboxylate

This compound was prepared substantially as described in Example 5 except starting from 4-methyl 1-[(3S)-tetrahydrofuran-3-yl] 4-[(chlorosulfonyl)methyl]piperidine-1,4-dicarboxylate and 4-(2,5-difluorophenyl)piperazine. LC-MS: 533.2 (M+H)⁺

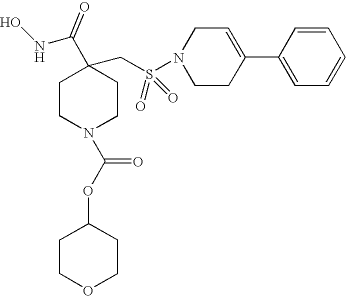

### Example 52

### 4-Hydroxycarbamoyl-4-(4-phenyl-3,6-dihydro-2H-pyridine-1-sulfonylmethyl)piperidine-1-carboxylic acid tetrahydropyran-4-yl ester

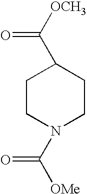 Step A
Dimethyl piperidine-1,4-dicarboxylate

To a solution of methyl piperidine-4-carboxylate (2.0 mL, 0.015 mol) and N,N-diisopropylethylamine (6.4 mL, 0.037 mol) in methylene chloride (50 mL, 0.8 mol) was added methyl chloroformate (1.4 mL, 0.018 mol) at 0° C. The reaction mixture was warmed to room temperature and stirred overnight. The mixture was diluted with methylene chloride and the reaction mixture was washed with 1 N HCl, water and brine successively, dried, and concentrated to provide the desired product, which was used without further purification. LC-MS: 202.0 (M+H)⁺. 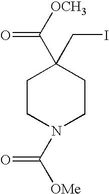
Step B
Dimethyl 4-(iodomethyl)piperidine-1,4-dicarboxylate

To a solution of lithium diisopropylamide (3.3 mL, 1.8 M in tetrahydrofuran) in THF (20 mL) was slowly added a solution of dimethyl piperidine-1,4-dicarboxylate (1.1 g, 0.0055 mol) in THF (5 mL) at −60° C., and the resultant solution was stirred for 30 min. Diiodomethane (0.53 mL, 0.0066 mol) was slowly added to the above solution at −60° C. and the mixture was stirred at room temperature overnight. The reaction was quenched with saturated NH₄Cl and the reaction mixture extracted with ethyl acetate. The organic phase was washed with water, dried over sodium sulfate, filtered and concentrated. The crude product was used in the next step without further purification. LC-MS: 342.0 (M+H)⁺. 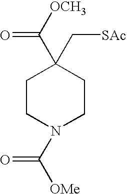
Step C
Dimethyl 4-[(acetylthio)methyl]piperidine-1,4-dicarboxylate

To a solution of dimethyl 4-(iodomethyl)piperidine-1,4-dicarboxylate (1.7 g, 0.0050 mol) in N,N-dimethylformamide (20 mL, 0.2 mol) was added potassium thioacetate (0.85 g, 0.0075 mol) at room temperature, and the mixture was stirred overnight. The mixture was diluted with water and extracted with ethyl acetate. The organic extract was washed with water and brine successively, dried over sodium sulfate, filtered and concentrated. The product was purified by CombiFlash using hexane/EtOAc (max. EtOAc 30%). LC-MS: 290.1 (M+H)⁺. 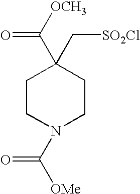
Step D
Dimethyl 4-[(chlorosulfonyl)methyl]piperidine-1,4-dicarboxylate.

To a solution of dimethyl 4-[(acetylthio)methyl]piperidine-1,4-dicarboxylate (0.9 g, 0.003 mol) in methylene chloride (5.0 mL, 0.078 mol) were added water (25 mL, 1.4 mol) and acetic acid (2.0 mL, 0.035 mol). Chlorine gas was bubbled into the above mixture at 0° C. for 30 min. After stirring for an additional 30 min., the mixture was diluted with methylene chloride, and layers were separated. The organic layer was washed with water and brine succesively, dried and concentrated to give the product, which was used without further purification. 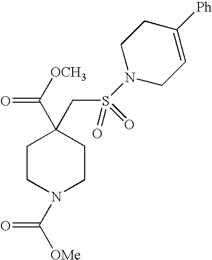
Step E
Dimethyl 4-[(4-phenyl-3,6-dihydropyridin-1(2H)-yl)sulfonyl]methylpiperidine-1,4-dicarboxylate

To a solution of dimethyl 4-[(chlorosulfonyl)methyl]piperidine-1,4-dicarboxylate (0.240 g, 0.000765 mol) and N,N-diisopropylethylamine (0.33 mL, 0.0019 mol) in methylene chloride (3.0 mL, 0.047 mol) was added 4-phenyl-1,2,3,6-tetrahydropyridine hydrochloride (0.19 g, 0.00084 mol) at 0° C., and the reaction mixture was stirred at this temperature for 1 h and then at room temperature for 1 h. The mixture was diluted with ethyl acetate, washed with 1 N HCl, water and brine successively, dried over sodium sulfate, filtered and concentrated. The product was purified by CombiFlash using CH₂Cl₂/EtOAc (max. EtOAc 20%) to provide the desired product. LC-MS: 437.1 (M+H)⁺, 895.1 (2M+Na)⁺. 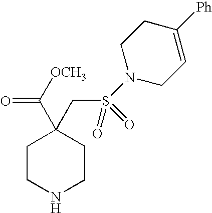
Step F
Methyl 4-[(4-phenyl-3,6-dihydropyridin-1(2H)-yl)sulfonyl]methylpiperidine-4-carboxylate

Into a 1-neck round-bottom flask equipped with a magnetic stir bar were added dimethyl 4-[(4-phenyl-3,6-dihydropyridin-1(2H)-yl)sulfonyl]methyl-piperidine-1,4-dicarboxylate (423 mg, 0.000969 mol) and CH₂Cl₂ (20 mL), followed by iodotrimethylsilane (414 uL, 0.00291 mol). The resulting solution was heated at 50° C. for 2.5 hours. The reaction mixture was diluted with CH₂Cl₂ (20 mL) and cooled to 0° C., followed by a careful addition of MeOH (ca. 1 mL) to quench the reaction. The resulting mixture then was diluted with 10 mL of H₂O (which made the solution cloudy), followed by 10 mL of saturated NaHCO₃ aqueous solution (which upon standing cleared and neutralized the solution). The organic layer was separated from the aqueous layer and washed with saturated NaHCO₃ (10 mL). The aquoeus layers were combined and extracted with DCM (2×5 mL) and then the combined organic phases were washed sequentially with H₂O (5 mL) and brine (5 mL), dried (Na₂SO₄), and was concentrated in-vacuo to afford a yellow reidue (418 mg). This residue was placed under vacuum overnight and then used directly in the next step without further purification. LC/MS: 379.1 (M+H)⁺; 757 (2M+H)⁺. 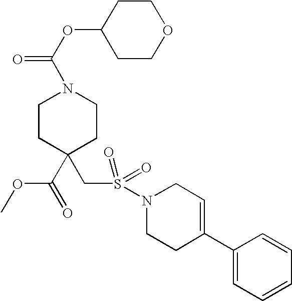
Step G
4-Methyl 1-(tetrahydro-2H-pyran-4-yl) 4-[(4-phenyl-3,6-dihydropyridin-1(2H)-yl)sulfonyl]methylpiperidine-1,4-dicarboxylate

To a solution of methyl 4-[(4-phenyl-3,6-dihydropyridin-1(2H)-yl)sulfonyl]methylpiperidine-4-carboxylate (0.0372 g, 0.0000982 mol) and 4-THP 4-nitrophenyl carbonate (0.025 g, 0.000094 mol) in dimethyl sulfoxide (0.15 mL, 0.0021 mol) was added N,N-diisopropyl-ethylamine (0.032 mL, 0.00019 mol), and the mixture was stirred at 90° C. for 2 h. The mixture was diluted with ethyl acetate and washed with diluted NaOH, water and brine successively, dried over sodium sulfate, filtered and concentrated. The product was purified by CombiFlash using CH₂Cl₂/EtOAc (max. EtOAc 30%) to provide the desired product. LC-MS: 507.2 (M+H)⁺. 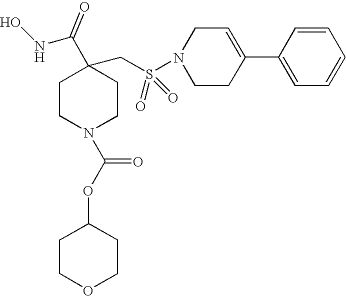
Step H
4-Hydroxycarbamoyl-4-(4-phenyl-3,6-dihydro-2H-pyridine-1-sulfonylmethyl)-piperidine-1-carboxylic acid tetrahydropyran-4-yl ester

This compound was prepared substantially starting from 4-methyl 1-(tetrahydro-2H-pyran-4-yl) 4-[(4-phenyl-3,6-dihydropyridin-1(2H)-yl)-sulfonyl]methylpiperidine-1,4-dicarboxylate using a procedure that was analogous to that described for the synthesis of Example 1, steps I and J. LC-MS: 508.2 (M+H)⁺

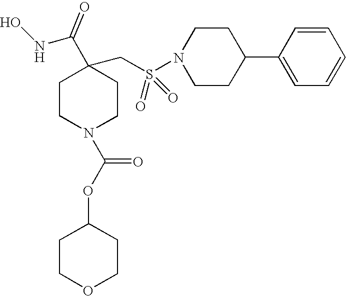

### Example 53

### 4-Hydroxycarbamoyl-4-(4-phenyl-piperidine-1-sulfonylmethyl)-piperidine-1-carboxylic acid tetrahydropyran-4-yl ester

This compound was prepared substantially using a procedure that was analogous to that described for the synthesis of Example 52 folowed by reduction of the double bond using the reaction conditions outlined in Example 2 starting from 4-hydroxycarbamoyl-4-(4-phenyl-3,6-dihydro-2H-pyridine-1-sulfonylmethyl)-piperidine-1-carboxylic acid tetrahydropyran-4-yl ester. LC-MS: 510.1 (M+H)⁺

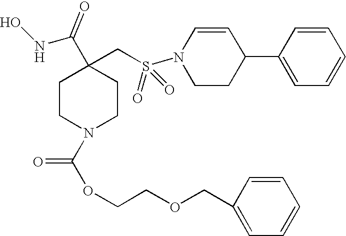

### Example 54

### 4-Hydroxycarbamoyl-4-(4-phenyl-3,6-dihydro-2H-pyridine-1-sulfonylmethyl)-piperidine-1-carboxylic acid 2-benzyloxyethyl ester

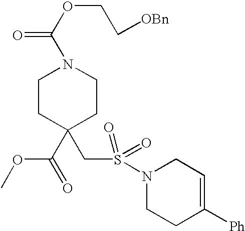 Step A
1-[2-(Benzyloxy)ethyl] 4-methyl 4-[(4-phenyl-3,6-dihydropyridin-1(2H)-yl)sulfonyl]methylpiperidine-1,4-dicarboxylate.

To a solution of methyl 4-[(4-phenyl-3,6-dihydro-pyridin-1(2H)-yl)sulfonyl]methylpiperidine-4-carboxylate (0.030 g, 0.000079 mol) (Example 52, Step F) and N,N-diisopropylethylamine (41 μL, 0.00024 mol) in methylene chloride (1.0 mL, 0.016 mol) was added 2-benzyloxyethyl chloroformate (17 μL, 0.000095 mol) at 0° C., and the mixture was stirred for 1 h at this temperature. The mixture was diluted with ethyl acetate, washed with 1 N HCl, water and brine successively, dried and concentrated. The product was purified by CombiFlash using CH₂Cl₂/EtOAc (max. EtOAc 30%). LC-MS: 557.3 (M+H)⁺. 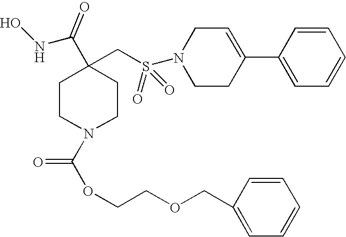
Step B
4-Hydroxycarbamoyl-4-(4-phenyl-3,6-dihydro-2H-pyridine-1-sulfonylmethyl)-piperidine-1-carboxylic acid 2-benzyloxyethyl ester

This compound was prepared from 1-[2-(benzyloxy)ethyl] 4-methyl 4-[(4-phenyl-3,6-dihydropyridin-1(2H)-yl)sulfonyl]-methylpiperidine-1,4-dicarboxylate using a procedure analogous to that described in steps I and J of Example 1. LC-MS: 558.3 (M+H)⁺

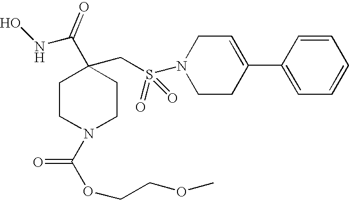

### Example 55

### 4-Hydroxycarbamoyl-4-(4-phenyl-3,6-dihydro-2H-pyridine-1-sulfonylmethyl)-piperidine-1-carboxylic acid 2-methoxyethyl ester

This compound was prepared substantially as described in Example 54 except starting from methyl 4-[(4-phenyl-3,6-dihydropyridin-1(2H)-yl)sulfonyl]methylpiperidine-4-carboxylate and 2-methoxyethyl chloridocarbonate. LC-MS: 482.2 (M+H)⁺

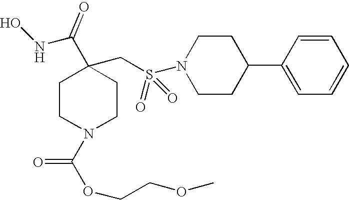

### Example 56

### 4-Hydroxycarbamoyl-4-(4-phenyl-piperidine-1-sulfonylmethyl)-piperidine-1-carboxylic acid 2-methoxyethyl ester

This compound was prepared substantially as described in Example 54 followed by reduction of the styrene double bond using the conditions outlined in Example 2. LC-MS: 484.2 (M+H)⁺

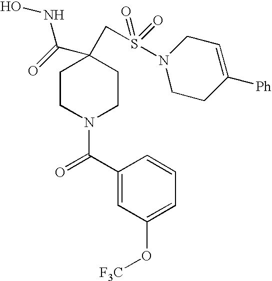

### Example 57

### N-Hydroxy-4-[(4-phenyl-3,6-dihydropyridin-1(2H)-yl)sulfonyl]methyl-1-[3-(trifluoromethoxy)benzoyl]piperidine-4-carboxamide

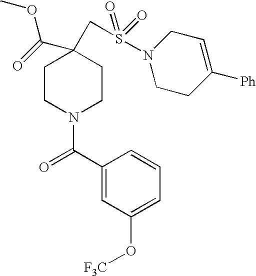 Step A
Methyl 4-[(4-phenyl-3,6-dihydropyridin-1(2H)-yl)sulfonyl]methyl-1-[3-(trifluoromethoxy)benzoyl]piperidine-4-carboxylate

Into a 1-neck round-bottom flask were added methyl 4-[(4-phenyl-3,6-dihydropyridin-1(2H)-yl)sulfonyl]methylpiperidine-4-carboxylate (16 mg, 0.000042 mol) and DMF (anhydrous, 1 mL) and to this solution were added sequentially benzotriazol-1-yloxytris(dimethylamino)phosphonium hexafluorophosphate (24 mg, 0.000054 mol), 3-(trifluoromethoxy)benzoic acid (1.0E1 mg, 0.000047 mol), and 4-methylmorpholine (9.4 μL, 0.000085 mol). The reaction mixture was stirred for 2 h. HPLC and LC/MS data suggested that the amine had been completely converted to the desired amide. The reaction mixture was diluted with EtOAc (20 mL), washed sequentially with saturated NaHCO₃ (3×2 mL) and brine (3 mL), dried (Na₂SO₄) and filtered. After filtration the volatiles were removed in-vacuo. The crude yellow product was purified by CombiFlash chromatography utilizing a 12 g column and eluting with EtOAc/CH₂Cl₂ (30% gradient over 15 min.) to afford 15 mg (63%) of pure product. LC/MS: 567.6 (M+H)⁺. 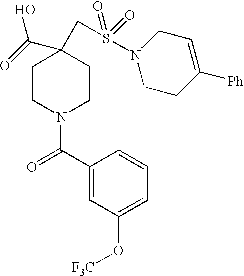
Step B
4-[(4-Phenyl-3,6-dihydropyridin-1(2H)-yl)sulfonyl]methyl-1-[3-(trifluoromethoxy)benzoyl]piperidine-4-carboxylic acid.

This compound was prepared substantially by using a procedure that was analogous to that described for the synthesis of Example 1, step I. LC/MS: 553.6 (M+H)⁺. 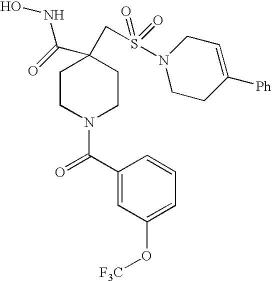
Step C
N-hydroxy-4-[(4-phenyl-3,6-dihydropyridin-1(2H)-yl)sulfonyl]methyl-1-[3-(trifluoromethoxy)benzoyl]piperidine-4-carboxamide

This compound was prepared substantially by using a procedure that was analogous to that described for the synthesis of Example 1, step J. LC/MS: 568.6 (M+H)⁺.

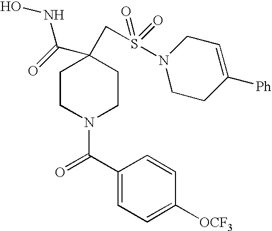

### Example 58

### N-Hydroxy-4-[(4-phenyl-3,6-dihydropyridin-1(2H)-yl)sulfonyl]methyl-1-[4-(trifluoromethoxy)benzoyl]piperidine-4-carboxamide

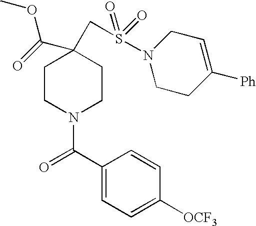 Step A
Methyl 4-[(4-phenyl-3,6-dihydropyridin-1(2H)-yl)sulfonyl]methyl-1-[4-(trifluoromethoxy)benzoyl]piperidine-4-carboxylate

Into a 1-neck round-bottom flask were added methyl 4-[(4-phenyl-3,6-dihydropyridin-1(2H)-yl)sulfonyl]methylpiperidine-4-carboxylate (16 mg, 0.000042 mol) and THF (1 mL) and to this solution were added N,N-diisopropylethylamine (22 μL, 0.00013 mol) and 4-(trifluoromethoxy)benzoyl chloride (8.0 uL, 0.000051 mol). After stirring for 2 h, HPLC and LC/MS data suggested that the reaction was complete. The reaction mixture was diluted with EtOAc (20 mL), washed sequentially with saturated 1 N HCl (3×2 mL) and brine (3 mL), dried (Na₂SO₄) and filtered. After filtration the volatiles were removed in-vacuo. The crude yellow product was purified by combiflash chromatography utilizing a 12 g column and eluting with EtOAc/CH₂Cl₂ (30% gradient over 15 min.) to afford 18 mg of pure product. LC/MS 567.6 (M+H)⁺. 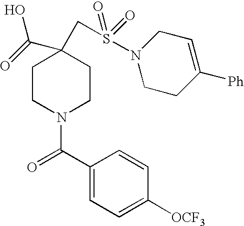
Step B
4-[(4-Phenyl-3,6-dihydropyridin-1(2H)-yl)sulfonyl]methyl-1-[4-(trifluoromethoxy)benzoyl]piperidine-4-carboxylic acid

This compound was prepared substantially by using a procedure that was analogous to that described for the synthesis of Example 1, step I. LC/MS 553.6 (M+H)⁺. 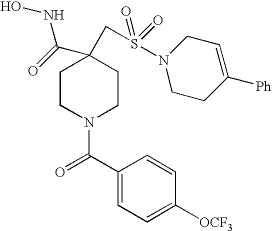
Step C
N-Hydroxy-4-[(4-phenyl-3,6-dihydropyridin-1(2H)-yl)sulfonyl]methyl-1-[4-(trifluoromethoxy)benzoyl]piperidine-4-carboxamide

This compound was prepared substantially by using a procedure that was analogous to that described for the synthesis of Example 1, step J. LC/MS 568.6 (M+H)⁺.

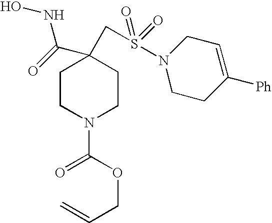

### Example 59

### Prop-2-en-1-yl 4-[(hydroxyamino)carbonyl]-4-{[(4-phenyl-3,6-dihydropyridin-1(2H)-yl)sulfonyl]methyl}piperidine-1-carboxylate

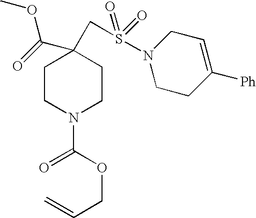 Step A
1-Allyl 4-methyl 4-[(4-phenyl-3,6-dihydropyridin-1(2H)-yl)sulfonyl]methylpiperidine-1,4-dicarboxylate

This compound was prepared substantially as described in Step A of Example 58, except starting with methyl 4-[(4-phenyl-3,6-dihydropyridin-1(2H)-yl)sulfonyl]methylpiperidine-4-carboxylate and allyl chloroformate. LC/MS 463.6 (M+H)⁺. 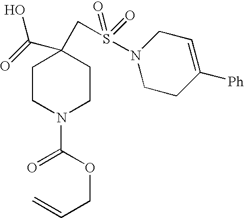
Step B
1-[(Allyloxy)carbonyl]-4-[(4-phenyl-3,6-dihydropyridin-1(2H)-yl)sulfonyl]methylpiperidine-4-carboxylic acid.

This compound was prepared substantially by using a procedure that was analogous to that described for the synthesis of Example 1, step I. LC/MS 449.6 (M+H)⁺. 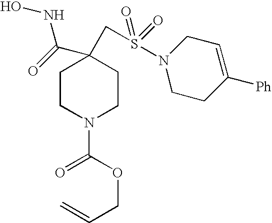
Step C
Prop-2-yn-1-yl 4-[(hydroxyamino)carbonyl]-4-[(4-phenyl-3,6-dihydropyridin-1(2H)-yl)sulfonyl]methylpiperidine-1-carboxylate

This compound was prepared substantially by using a procedure that was analogous to that described for the synthesis of Example 1, step J. LC/MS 464.6 (M+H)⁺.

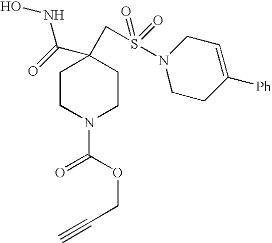

### Example 60

### Prop-2-yn-1-yl 4-[(hydroxyamino)carbonyl]-4-[(4-phenyl-3,6-dihydropyridin-1(2H)-yl)sulfonyl]methylpiperidine-1-carboxylate

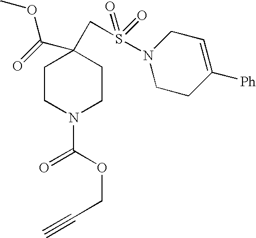 Step A
4-Methyl 1-prop-2-yn-1-yl 4-[(4-phenyl-3,6-dihydropyridin-1(2H)-yl)sulfonyl]methylpiperidine-1,4-dicarboxylate

This compound was prepared substantially as described in Step A of Example 58, except starting with prop-2-yn-1-yl chloridocarbonate. LC/MS 460.6 (M+H)⁺. 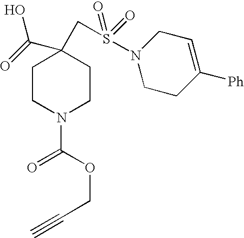
Step B
4-[(4-Phenyl-3,6-dihydropyridin-1(2H)-yl)sulfonyl]methyl-1-[(prop-2-yn-1-yloxy)carbonyl]piperidine-4-carboxylic acid.

This compound was prepared substantially by using a procedure that was analogous to that described for the synthesis of Example 1, step K. LC/MS 460.6 (M+H)⁺. 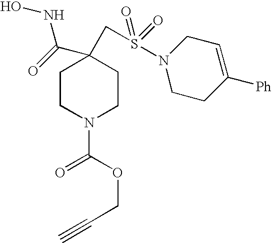
Step C
Prop-2-yn-1-yl 4-[(hydroxyamino)carbonyl]-4-[(4-phenyl-3,6-dihydropyridin-1(2H)-yl)sulfonyl]methylpiperidine-1-carboxylate

This compound was prepared substantially by using a procedure that was analogous to that described for the synthesis of Example 1, step J. LC/MS 462.6 (M+H)⁺.

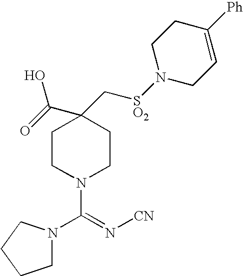

### Example 61

### 1-[(Cyanoimino)(pyrrolidin-1-yl)methyl]-N-hydroxy-4-{[(4-phenyl-3,6-dihydropyridin-1(2H)-yl)sulfonyl]methyl}piperidine-4-carboxamide

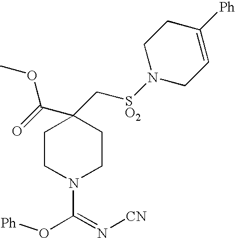 Step A
Methyl 1-[(cyanoimino)(phenoxy)methyl]-4-{[(4-phenyl-3,6-dihydropyridin-1(2H)-yl)sulfonyl]methyl}piperidine-4-carboxylate

Into a 2-neck-round-bottom flask were added methyl 4-{[(4-phenyl-3,6-dihydropyridin-1(2H)-yl)sulfonyl]methyl}piperidine-4-carboxylate (183 mg, 0.000484 mol) in anhydrous acetonitrile (10 mL, 0.0484 M), N,N-diisopropylethylamine (450 uL, 0.0026 mol), and diphenyl cyanocarbonimidate (230 mg, 0.00096 mol). The solution was heated to 90° C. and the reaction was monitored by HPLC and LC/MS. After stirring for 16 h, the reaction mixture was allowed to cool to ambient temperature and was concentrated in-vacuo. The crude product was purified by CombiFlash chromatography utilizing a 12 g column and eluting with EtOAc/CH₂Cl₂ (30% gradient over 15 min.) to afford 240 mg (94%) of pure product. LC/MS: 523.6 (M+H)⁺. 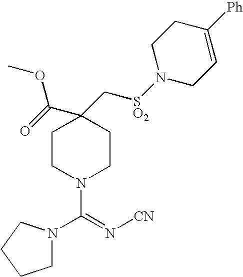
Step B
Methyl 1-[(cyanoimino)(pyrrolidin-1-yl)methyl]-4-{[(4-phenyl-3,6-dihydropyridin-1(2H)-yl)sulfonyl]methyl}piperidine-4-carboxylate

To a solution of methyl 1-[(cyanoimino)(phenoxy)methyl]-4-{[(4-phenyl-3,6-dihydropyridin-1(2H)-yl)sulfonyl]methyl}piperidine-4-carboxylate (20 mg, 0.04 mmol) in anhydrous acetonitrile (2.0 mL) was added pyrrolidine (16 uL, 0.19 mmol, 5.0 equiv.) and the solution was refluxed for 16 h. The reaction mixture was allowed to cool to ambient temperature and was concentrated in-vacuo. The crude product was purified by CombiFlash chromatography utilizing a 12 g column and eluting with EtOAc/CH₂Cl₂ (30% gradient over 15 min.) to afford the pure product (16 mg, 80%). LC/MS: 500.6 (M+H)⁺. 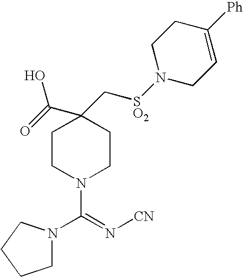
Step C
1-[(Cyanoimino)pyrrolidin-1-yl)methyl]-4-{[(4-phenyl-3,6-dihydropyridin-1(2H)-yl)sulfonyl]methyl}piperidine-4-carboxylic acid

This compound was prepared substantially by using a procedure that was analogous to that described for the synthesis of Example 1, step I. LC-MS: 486.2 (M+H)⁺. 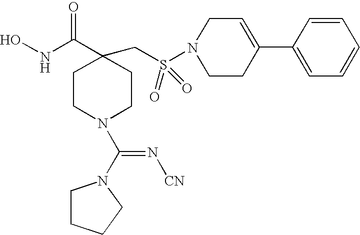
Step D
1-[(Cyanoimino)(pyrrolidin-1-yl)methyl]-N-hydroxy-4-{[(4-phenyl-3,6-dihydropyridin-1(2H)-yl)sulfonyl]methyl}piperidine-4-carboxamide

This compound was prepared substantially by using a procedure that was analogous to that described for the synthesis of Example 1, step J. LC-MS: 501.1 (M+H)⁺.

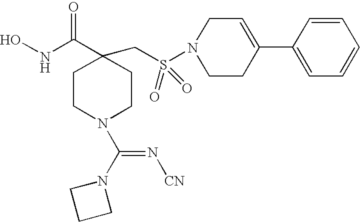

### Example 62

### 1-[Azetidin-1-yl(cyanoimino)methyl]-N-hydroxy-4-{[(4-phenyl-3,6-dihydropyridin-1(2H)-yl)sulfonyl]methyl}piperidine-4-carboxamide

This compound was prepared substantially as described in Example 61 except using methyl 1-[(cyanoimino)(phenoxy)methyl]-4-{[(4-phenyl-3,6-dihydropyridin-1(2H)-yl)sulfonyl]methyl}piperidine-4-carboxylate and azetidine hydrochloride. LC-MS: 487.6 (M+H)⁺.

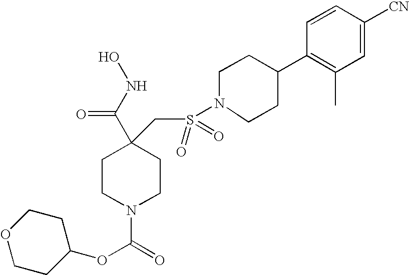

### Example 63

### Tetrahydro-2H-pyran-4-yl 4-({[4-(4-cyano-2-methylphenyl)piperidin-1-yl]sulfonyl}methyl)-4-[(hydroxyamino)carbonyl]piperidine-1-carboxylate

This compound was prepared substantially by using a procedure analogous to that described for the synthesis of Example 52, followed by reduction of the double bond using a procedure similar to that outlined in Examle 2. LC-MS: 549.6 (M+H)⁺.

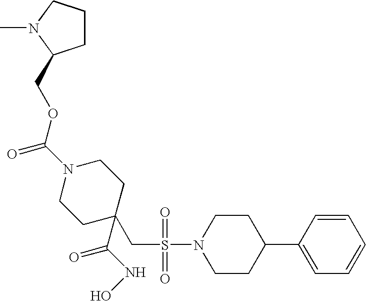

### Example 64

### [(2S)-1-Methylpyrrolidin-2-yl]methyl 4-[(hydroxyamino)carbonyl]-4-{[(4-phenyl-3,6-dihydropyridin-1(2H)-yl)sulfonyl]methyl}piperidine-1-carboxylate

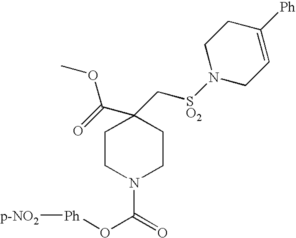 Step A
4-Methyl 1-(4-nitrophenyl) 4-{[(4-phenyl-3,6-dihydropyridin-1(2H)-yl)sulfonyl]methyl}piperidine-1,4-dicarboxylate.

To a solution of p-nitrophenyl chloroformate (220 mg, 1.1 mmol) and N,N-diisopropylethylamine (500 uL, 3.0 mmol) in anhydrous acetonitrile (5 mL) was added a solution of methyl 4-{[(4-phenyl-3,6-dihydropyridin-1(2H)-yl)sulfonyl]methyl}piperidine-4-carboxylate (366 mg, 0.976 mmol) in anhydrous acetonitrile (2 mL) at 0° C. The reaction mixture was allowed to warm to ambient temperature over 1 h and the reaction was monitored by HPLC and LC/MS. After stirring for 2 h, the reaction mixture was concentrated in-vacuo and the crude product was purified by CombiFlash chromatography utilizing a 12 g column and eluting with EtOAc/CH₂Cl₂ (30% gradient over 15 min.) to afford the product (426 mg, 81%). LC/MS: 544.6 (M+H)⁺. 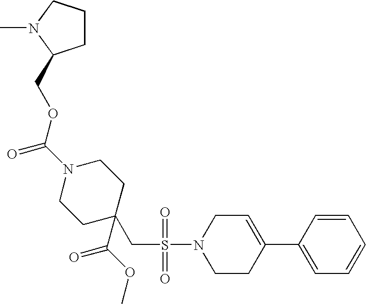
Step B
4-Methyl 1-{[(2S)-1-methylpyrrolidin-2-yl]methyl} 4-{[(4-phenyl-3,6-dihydropyridin-1(2H)-yl)sulfonyl]methyl}piperidine-1,4-dicarboxylate.

To a 0° C. solution of 4-methyl 1-(4-nitrophenyl) 4-{[(4-phenyl-3,6-dihydropyridin-1(2H)-yl)sulfonyl]methyl}piperidine-1,4-dicarboxylate (30 mg, 0.06 mmol) in anhydrous THF (1 mL) were added N-methyl-L-prolinol (7.0 uL, 0.06 mmol) and sodium hydride (4.4 mg, 0.12 mmol) and the solution was allowed to gradually warm to ambient temperature over 1 h. The reaction was monitored by HPLC and LC/MS. After stirring for 2 h, the reaction mixture was concentrated in-vacuo and the crude product purified by CombiFlash chromatography utilizing a 12 g column and eluting with MeOH/CH₂Cl₂ (20% gradient over 15 min.) to afford the product (28 mg, 94%). LC/MS: 520.6 (M+H)⁺. 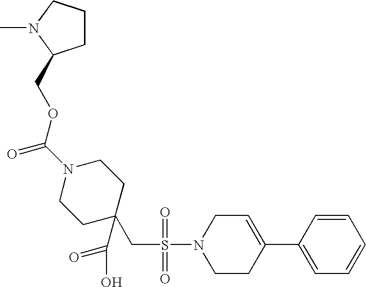
Step C
1-({[(2S)-1-Methylpyrrolidin-2-yl]methoxy}carbonyl)-4-{[(4-phenyl-3,6-dihydropyridin-1(2H)-yl)sulfonyl]methyl}piperidine-4-carboxylic acid.

This compound was prepared substantially by using a procedure that was analogous to that described for the synthesis of Example 1, step I. LC-MS: 506.6 (M+H)⁺. 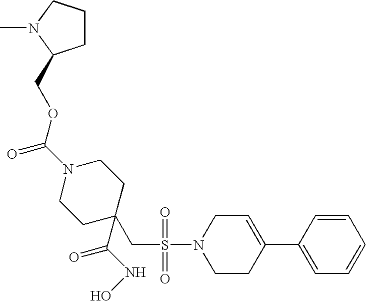
Step D
[(2S)-1-Methylpyrrolidin-2-yl]methyl 4-[(hydroxyamino)carbonyl]-4-{[(4-phenyl-3,6-dihydropyridin-1(2H)-yl)sulfonyl]methyl}piperidine-1-carboxylate

This compound was prepared substantially by using a procedure that was analogous to that described for the synthesis of Example 1, step J. LC-MS: 521.2 (M+H)⁺.

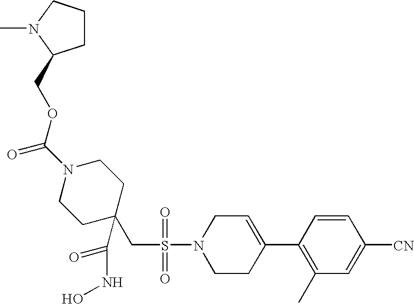

### Example 65

### ((2S)-1-Methylpyrrolidin-2-yl)methyl 4-({[4-(4-cyano-2-methylphenyl)piperidin-1-yl]sulfonyl}methyl)-4-[(hydroxyamino)carbonyl]piperidine-1-carboxylate

This compound was prepared substantially using a procedure analogous to that described for the synthesis of Example 64. LC-MS: 560.6 (M+H)⁺.

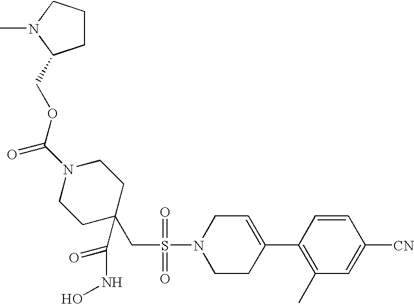

### Example 66

### ((2R)-Pyrrolidin-2-yl)methyl 4-({[4-(4-cyano-2-methylphenyl)piperidin-1-yl]sulfonyl}methyl)-4-[(hydroxyamino)carbonyl]piperidine-1-carboxylate

This compound was prepared substantially by using a procedure that was analogous to that described for the synthesis of Example 64. LC-MS: 560.6 (M+H)⁺.

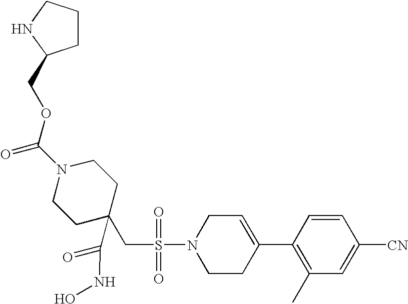

### Example 67

### (2S)-Pyrrolidin-2-ylmethyl 4-({[4-(4-cyano-2-methylphenyl)-3,6-dihydropyridin-1(2H)-yl]sulfonyl}methyl)-4-[(hydroxyamino)carbonyl]piperidine-1-carboxylate

This compound was prepared substantially by using a procedure that was analogous to that described for the synthesis of Example 64. LC-MS: 546.6 (M+H)⁺.

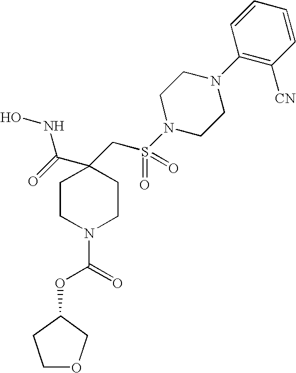

### Example 68

### (3S)-Tetrahydrofuran-3-yl 4-({[4-(2-cyanophenyl)piperazin-1-yl]sulfonyl}methyl)-4-[(hydroxyamino)carbonyl]piperidine-1-carboxylate

This compound was prepared by using a procedure analogous to that described for the synthesis of example 5. LC-MS: 522.1 (M+H)⁺.

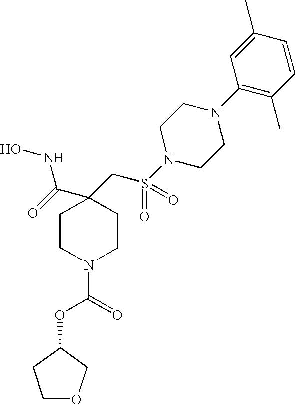

### Example 69

### (3S)-Tetrahydrofuran-3-yl 4-({[4-(2,5-dimethylphenyl)piperazin-1-yl]sulfonyl}methyl)-4-[(hydroxyamino)carbonyl]piperidine-1-carboxylate

This compound was prepared by using a procedure analogous to that described for the synthesis of example 5. LC-MS: 525.1 (M+H)⁺.

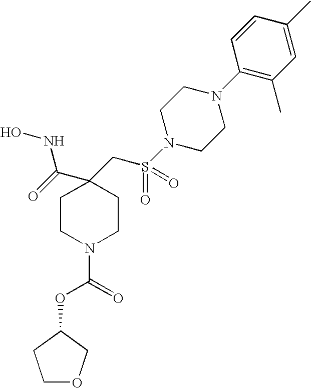

### Example 70

### (3S)-Tetrahydrofuran-3-yl 4-({[4-(2,4-dimethylphenyl)piperazin-1-yl]sulfonyl}methyl)-4-[(hydroxyamino)carbonyl]piperidine-1-carboxylate

This compound was prepared by using a procedure analogous to that described for the synthesis of example 5. LC-MS: 525.1 (M+H)⁺.

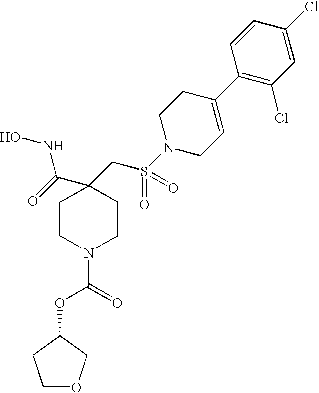

### Example 71

### (3S)-Tetrahydrofuran-3-yl 4-({[4-(2,4-dichlorophenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 1. LC-MS: 562.0 & 564.0 (M+H)⁺.

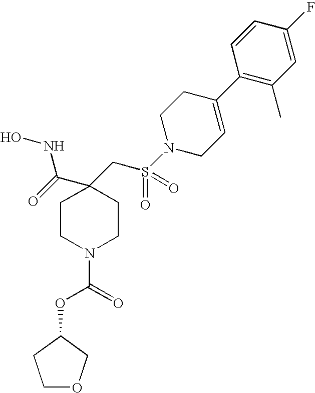

### Example 72

### (3S)-Tetrahydrofuran-3-yl 4-({[4-(4-fluoro-2-methylphenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 1. LC-MS: 526.0 (M+H)⁺.

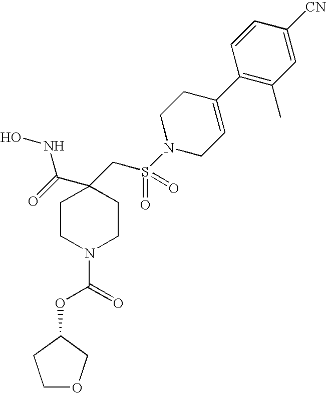

### Example 73

### (3S)-Tetrahydrofuran-3-yl 4-({[4-(4-cyano-2-methylphenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 1. LC-MS: 533.1 (M+H)⁺.

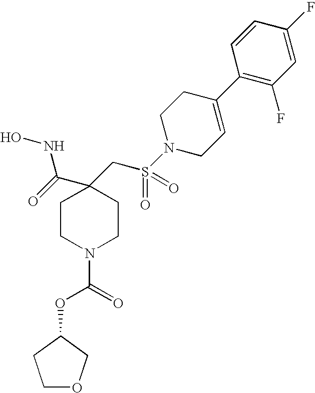

### Example 74

### (3S)-Tetrahydrofuran-3-yl 4-({[4-(2,4-difluorophenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 1. LC-MS: 530.0 (M+H)⁺.

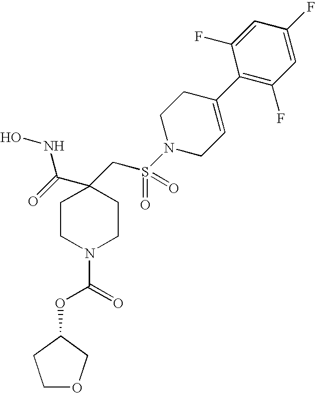

### Example 75

### (3S)-Tetrahydrofuran-3-yl 4-[(hydroxyamino)carbonyl]-4-({[4-(2,4,6-trifluorophenyl)-3,6-dihydropyridin-1(2H)-yl]sulfonyl}methyl)piperidine-1-carboxylate

This compound was prepared by using a procedure analogous to that described for the synthesis of example 1. LC-MS: 548.0 (M+H)⁺.

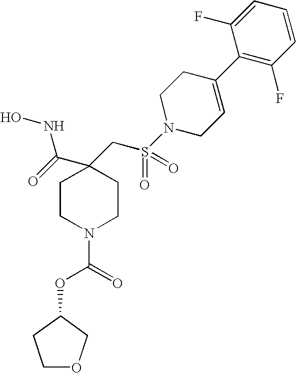

### Example 76

### (3S)-Tetrahydrofuran-3-yl 4-({[4-(2,6-difluorophenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 1. LC-MS: 530.0 (M+H)⁺.

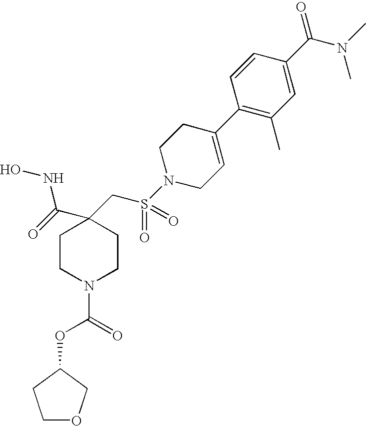

### Example 77

### (3S)-Tetrahydrofuran-3-yl 4-({[4-{4-[(dimethylamino)carbonyl]-2-methylphenyl}-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 1. LC-MS: 579.1 (M+H)⁺.

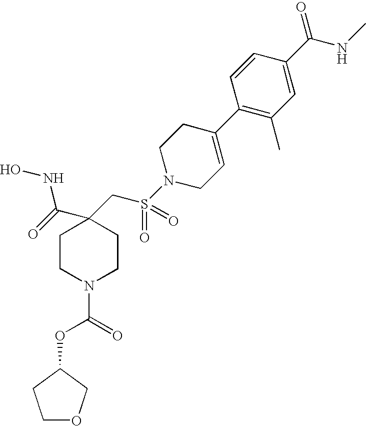

### Example 78

### (3S)-Tetrahydrofuran-3-yl 4-[(hydroxyamino)carbonyl]-4-({[4-{2-methyl-4-[(methylamino)carbonyl]phenyl}-3,6-dihydropyridin-1(2H)-yl]sulfonyl}methyl)piperidine-1-carboxylate

This compound was prepared by using a procedure analogous to that described for the synthesis of example 1. LC-MS: 565.1 (M+H)⁺.

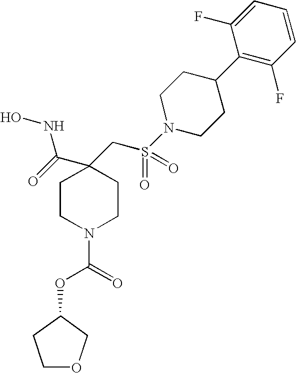

### Example 79

### (3S)-Tetrahydrofuran-3-yl 4-({[4-(2,6-difluorophenyl)piperidin-1-yl]sulfonyl}methyl)-4-[(hydroxyamino)carbonyl]piperidine-1-carboxylate

This compound was prepared by using a procedure analogous to that described for the synthesis of example 2. LC-MS: 532.2 (M+H)⁺.

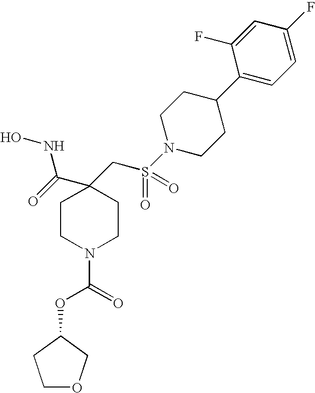

### Example 80

### (3S)-Tetrahydrofuran-3-yl 4-({[4-(2,4-difluorophenyl)piperidin-1-yl]sulfonyl}methyl)-4-[(hydroxyamino)carbonyl]piperidine-1-carboxylate

This compound was prepared by using a procedure analogous to that described for the synthesis of example 2. LC-MS: 532.1 (M+H)⁺.

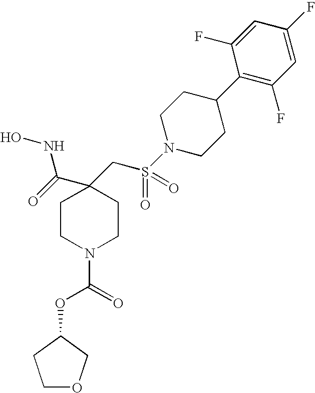

### Example 81

### (3S)-Tetrahydrofuran-3-yl 4-[(hydroxyamino)carbonyl]-4-({[4-(2,4,6-trifluorophenyl)piperidin-1-yl]sulfonyl}methyl)piperidine-1-carboxylate

This compound was prepared by using a procedure analogous to that described for the synthesis of example 2. LC-MS: 550.0 (M+H)⁺.

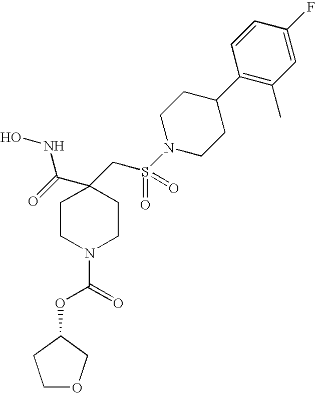

### Example 82

### (3S)-Tetrahydrofuran-3-yl 4-({[4-(4-fluoro-2-methylphenyl)piperidin-1-yl]sulfonyl}methyl)-4-[(hydroxyamino)carbonyl]piperidine-1-carboxylate

This compound was prepared by using a procedure analogous to that described for the synthesis of example 2. LC-MS: 528.2 (M+H)⁺.

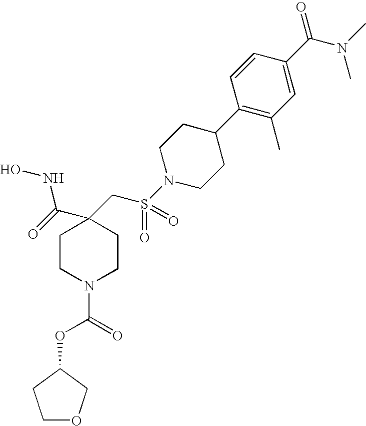

### Example 83

### (3S)-Tetrahydrofuran-3-yl 4-{[(4-{4-[(dimethylamino)carbonyl]-2-methylphenyl}piperidin-1-yl)sulfonyl]methyl}-4-[(hydroxyamino)carbonyl]piperidine-1-carboxylate

This compound was prepared by using a procedure analogous to that described for the synthesis of example 2. LC-MS: 528.2 (M+H)⁺.

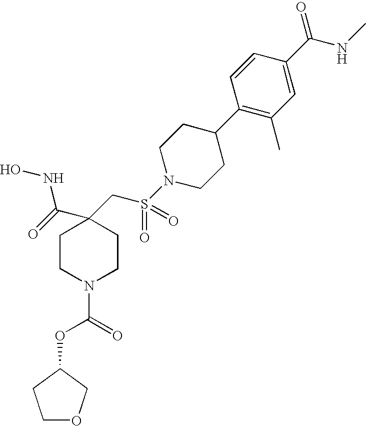

### Example 84

### (3S)-Tetrahydrofuran-3-yl 4-[(hydroxyamino)carbonyl]-4-{[(4-{2-methyl-4-[(methylamino)carbonyl]phenyl}piperidin-1-yl)sulfonyl]methyl}piperidine-1-carboxylate

This compound was prepared by using a procedure analogous to that described for the synthesis of example 2. LC-MS: 567.1 (M+H)⁺.

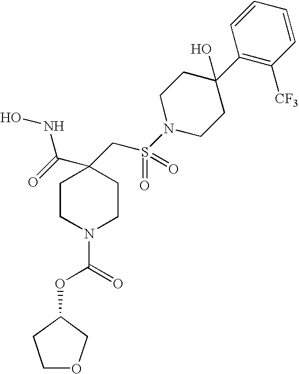

### Example 85

### (3S)-Tetrahydrofuran-3-yl 4-[(hydroxyamino)carbonyl]-4-[({4-hydroxy-4-[2-(trifluoromethyl)phenyl]piperidin-1-yl}sulfonyl)methyl]piperidine-1-carboxylate

This compound was prepared by using a procedure analogous to that described for the synthesis of example 1, with the exception that step F was replaced by the following procedure:

To a solution of 1-iodo-2-(trifluoromethyl)-benzene (0.72 g, 0.0026 mol) in tetrahydrofuran (10.0 mL, 0.123 mol) was slowly added 1.6 M of n-butyllithium in hexane (1.5 mL) at −70° C. and the mixture was stirred for 1 h. A solution of tert-butyl 4-oxo-1-piperidinecarboxylate (0.44 g, 0.0022 mol) in THF (1.0 mL) was added to the above solution at at −70° C. and the mixture was slowly warmed to rt and stirred for 5 h. The reaction was quenched with saturated NH₄Cl and extracted with diethyl ether, which was washed with brine, dried and concentrated. The product was purified by CombiFlash eluting with hexane/EtOAc (max. EA 30%).

The title compound was confirmed by LC-MS: 580.2 (M+H)⁺.

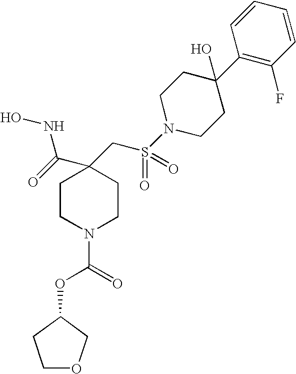

### Example 86

### (3S)-Tetrahydrofuran-3-yl 4-({[4-(2-fluorophenyl)-4-hydroxypiperidin-1-yl]sulfonyl}methyl)-4-[(hydroxyamino)carbonyl]piperidine-1-carboxylate

This compound was prepared by using a procedure analogous to that described for the synthesis of example 85. LC-MS: 530.1 (M+H)⁺.

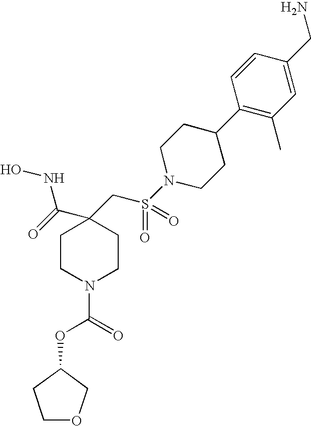

### Example 87

### (3S)-Tetrahydrofuran-3-yl 4-[({4-[4-(aminomethyl)-2-methylphenyl]piperidin-1-yl}sulfonyl)methyl]-4-[(hydroxyamino)carbonyl]piperidine-1-carboxylate

This compound was prepared by using a procedure analogous to that described for the synthesis of example 2. LC-MS: 530.1 (M+H)⁺.

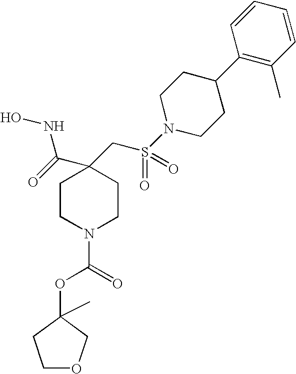

### Example 88

### 3-Methyltetrahydrofuran-3-yl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 524.2 (M+H)⁺.

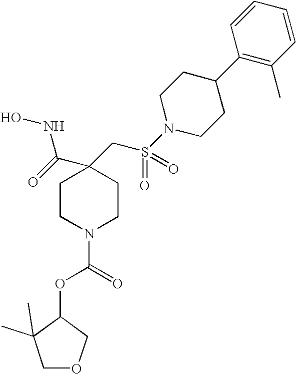

### Example 89

### 4,4-Dimethyltetrahydrofuran-3-yl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 538.2 (M+H)⁺.

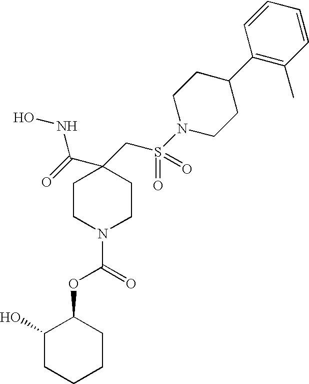

### Example 90

### trans-2-Hydroxycyclohexyl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 538.2 (M+H)⁺.

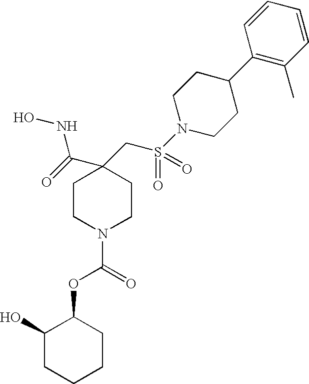

### Example 91

### cis-2-Hydroxycyclohexyl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 538.2 (M+H)⁺.

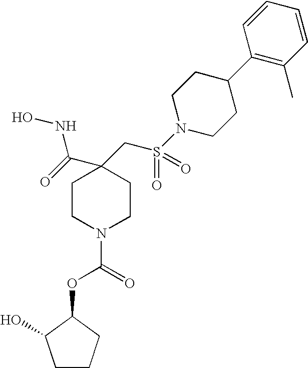

### Example 92

### trans-2-Hydroxycyclopentyl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 524.1 (M+H)⁺.

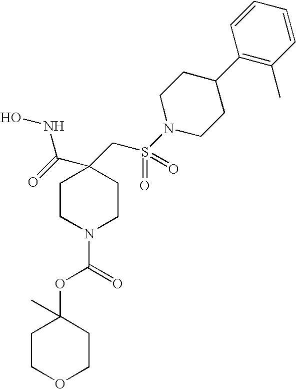

### Example 93

### 4-Methyltetrahydro-2H-pyran-4-yl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 396.1 (M-CO₂-4-Me-THP+H)⁺.

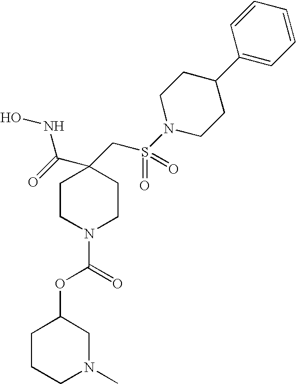

### Example 94

### 1-Methylpiperidin-3-yl 4-[(hydroxyamino)carbonyl]-4-({[4-phenyl-piperidin-1-yl]sulfonyl}methyl)piperidine-1-carboxylate

This compound was prepared by using a procedure analogous to that described for the synthesis of example 53. LC-MS: 523.1 (M+H)⁺.

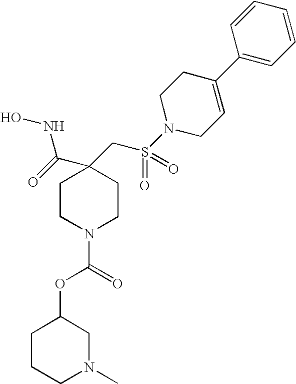

### Example 95

### 1-Methylpiperidin-3-yl 4-[(hydroxyamino)carbonyl]-4-({[4-phenyl-3,6-dihydropyridin-1(2H)-yl]sulfonyl}methyl)piperidine-1-carboxylate

This compound was prepared by using a procedure analogous to that described for the synthesis of example 52. LC-MS: 521.1 (M+H)⁺.

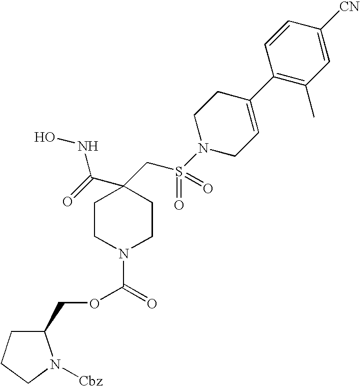

### Example 96

### {(2S)-1-[(Benzyloxy)carbonyl]pyrrolidin-2-yl}methyl 4-({[4-(4-cyano-2-methylphenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 52. LC-MS: 680.2 (M+H)⁺.

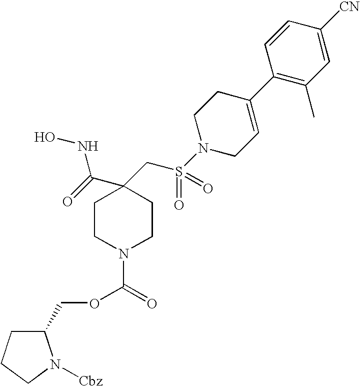

### Example 97

### {(2R)-1-[(Benzyloxy)carbonyl]pyrrolidin-2-yl}methyl-4-({[4-(4-cyano-2-methylphenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 52. LC-MS: 680.2 (M+H)⁺.

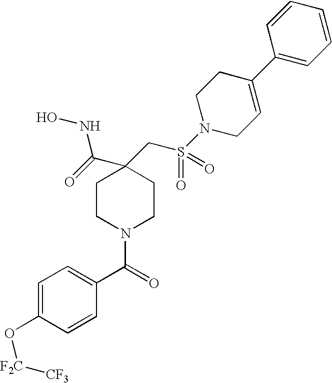

### Example 98

### N-Hydroxy-1-[4-(pentafluoroethoxy)benzoyl]-4-{[(4-phenyl-3,6-dihydropyridin-1(2H)-yl)sulfonyl]methyl}piperidine-4-carboxamide

This compound was prepared by using a procedure analogous to that described for the synthesis of example 58. LC-MS: 618.1 (M+H)⁺.

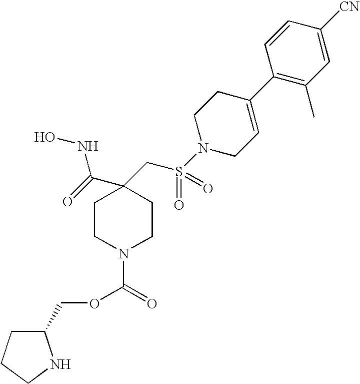

### Example 99

### (2R)-Pyrrolidin-2-ylmethyl 4-({[4-(4-cyano-2-methylphenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 52. LC-MS: 546.1 (M+H)⁺.

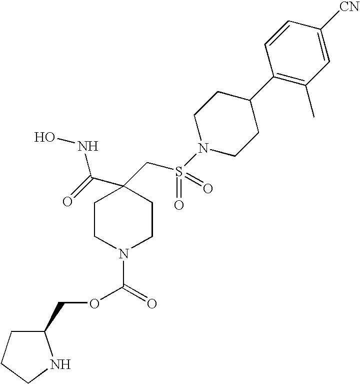

### Example 100

### (2S)-Pyrrolidin-2-ylmethyl 4-({[4-(4-cyano-2-methylphenyl)piperidin-1-yl]sulfonyl}methyl)-4-[(hydroxyamino)carbonyl]piperidine-1-carboxylate

This compound was prepared by using a procedure analogous to that described for the synthesis of example 53. LC-MS: 548.2 (M+H)⁺.

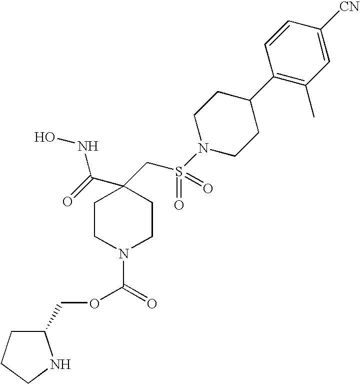

### Example 101

### (2R)-Pyrrolidin-2-ylmethyl 4-({[4-(4-cyano-2-methylphenyl)piperidin-1-yl]sulfonyl}methyl)-4-[(hydroxyamino)carbonyl]piperidine-1-carboxylate

This compound was prepared by using a procedure analogous to that described for the synthesis of example 53. LC-MS: 548.2 (M+H)⁺.

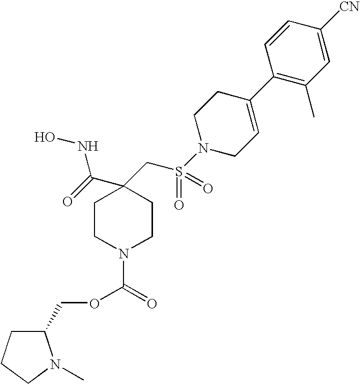

### Example 102

### [(2R)-1-Methylpyrrolidin-2-yl]methyl 4-({[4-(4-cyano-2-methylphenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 52. LC-MS: 560.2 (M+H)⁺.

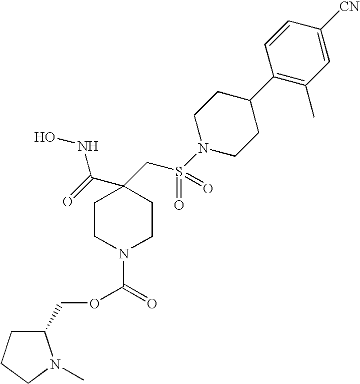

### Example 103

### [(2R)-1-Methylpyrrolidin-2-yl]methyl 4-({[4-(4-cyano-2-methylphenyl)piperidin-1-yl]sulfonyl}methyl)-4-[(hydroxyamino)carbonyl]piperidine-1-carboxylate

This compound was prepared by using a procedure analogous to that described for the synthesis of example 53. LC-MS: 562.1 (M+H)⁺.

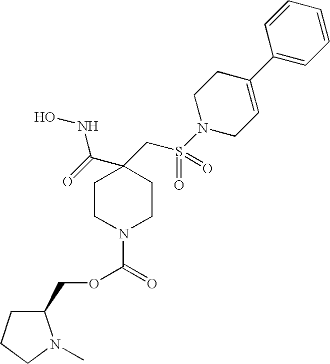

### Example 104

### [(2S)-1-Methylpyrrolidin-2-yl]methyl 4-[(hydroxyamino)carbonyl]-4-{[(4-phenyl-3,6-dihydropyridin-1(2H)-yl)sulfonyl]methyl}piperidine-1-carboxylate

This compound was prepared by using a procedure analogous to that described for the synthesis of example 52. LC-MS: 521.1 (M+H)⁺.

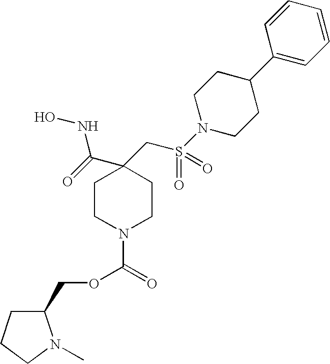

### Example 105

### [(2S)-1-Methylpyrrolidin-2-yl]methyl 4-[(hydroxyamino)carbonyl]-4-{[(4-phenylpiperidin-1-yl)sulfonyl]methyl}piperidine-1-carboxylate

This compound was prepared by using a procedure analogous to that described for the synthesis of example 53. LC-MS: 523.2 (M+H)⁺.

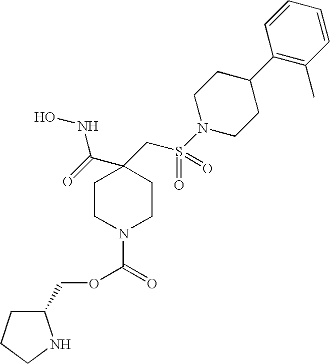

### Example 106

### (2R)-Pyrrolidin-2-ylmethyl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 523.2 (M+H)⁺.

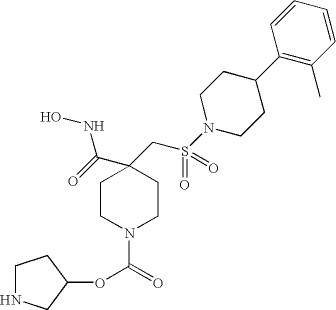

### Example 107

### Pyrrolidin-3-yl 4-[(hydroxyamino)carbonyl]-4-({[4-(2-methylphenyl)piperidin-1-yl]sulfonyl}methyl)piperidine-1-carboxylate

This compound was prepared by using a procedure analogous to that described for the synthesis of example 53. LC-MS: 509.2 (M+H)⁺.

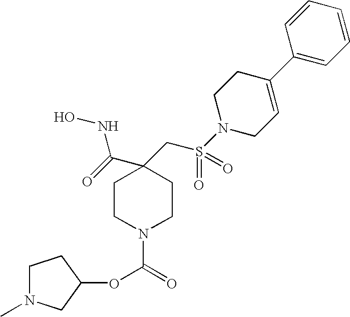

### Example 108

### 1-Methylpyrrolidin-3-yl 4-[(hydroxyamino)carbonyl]-4-{[(4-phenyl-3,6-dihydropyridin-1(2H)-yl)sulfonyl]methyl}piperidine-1-carboxylate

This compound was prepared by using a procedure analogous to that described for the synthesis of example 52. LC-MS: 507.1 (M+H)⁺.

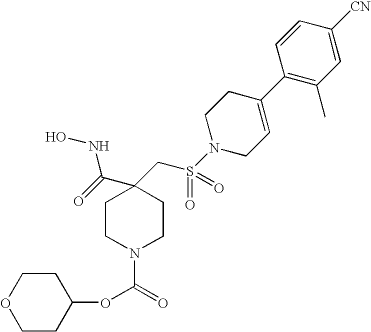

### Example 109

### Tetrahydro-2H-pyran-4-yl 4-({[4-(4-cyano-2-methylphenyl)-3,6-dihydropyridin-1(2H)-yl]sulfonyl}methyl)-4-[(hydroxyamino)carbonyl]piperidine-1-carboxylate

This compound was prepared by using a procedure analogous to that described for the synthesis of example 52. LC-MS: 547.1 (M+H)⁺.

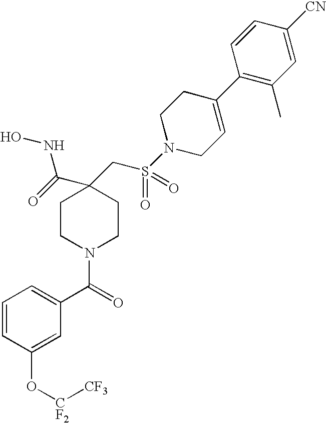

### Example 110

### 4-({[4-(4-Cyano-2-methylphenyl)-3,6-dihydropyridin-1(2H)-yl]sulfonyl}methyl)-N-hydroxy-1-[3-(pentafluoroethoxy)benzoyl]piperidine-4-carboxamide

This compound was prepared by using a procedure analogous to that described for the synthesis of example 57. LC-MS: 657.1 (M+H)⁺.

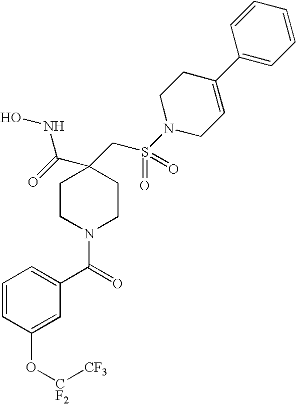

### Example 111

### N-Hydroxy-1-[3-(pentafluoroethoxy)benzoyl]-4-{[(4-phenyl-3,6-dihydropyridin-1(2H)-yl)sulfonyl]methyl}piperidine-4-carboxamide

This compound was prepared by using a procedure analogous to that described for the synthesis of example 57. LC-MS: 618.1 (M+H)⁺.

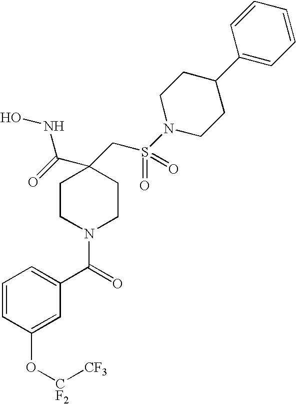

### Example 112

### N-Hydroxy-1-[3-(pentafluoroethoxy)benzoyl]-4-{[(4-phenylpiperidin-1-yl)sulfonyl]methyl}piperidine-4-carboxamide

This compound was prepared by using a procedure analogous to that described for the synthesis of example 57, followed by reduction of the double bond using a procedure similar to that outlined for the synthesis of example 2. LC-MS: 620.2 (M+H)⁺.

### Example A

The capacity of the compounds of the invention to inhibit metalloproteases can be determined using a suitable screen such as a high through-put assay. For example, an agent can be tested in an extracellular acidification assay, calcium flux assay, ligand binding assay or chemotaxis assay. Below are example assays.

TNFα assay

In some embodiments, the capacity of the compounds of the invention to act as inhibitors of the production of TNFα can be determined using the following procedure. A 100 μM solution of the inhibitor being tested or dilutions thereof is incubated at 37° C. in an atmosphere of 5% CO₂ with THP-1 cells (human monocytes) suspended in RPM1 1640 medium and 20 μM β-mercaptoethanol at a cell density of 1×10⁶/ml and stimulated with LPS. After 18 hours the supernatant is assayed for the levels of TNFα using a commercially available ELISA kit. The activity in the presence of 0.1 mM inhibitor or dilutions thereof is compared to activity in a control devoid of inhibitor and results reported as that inhibitor concentration effecting 50% inhibition of the production of TNFα.

PBMC Assay Measuring TNFα Activity

A leukophoresis is obtained from (Biological Specialties, Colmar Pa.) from normal drug free (no aspirin, ibuprofen, NSAIDs) etc.) donors. In a 50 mL conical tube (VWR, NJ), add 20 mL of blood and 20 mL of sterile 0.9% saline (Baxter Healthcare, Dearfield, Ill.) and mix well. Underlay 10 mL of endotoxin free ficoll paque (Pharmacia, Uppsala, Sweden) and spin at 3000 RPM for 30 minutes. Remove the layer of white blood cells and wash with 50 mls 0.9% saline. Count cells and add 0.250 mL to 96 well plate (Costar/Corning VWR, NJ) at 2×10 6 c/ml, in RPMI 1640 medium (Gibco BRL). Add compounds and preincubate with cells for 10 min before adding LPS (Calbiochem, Calif.) at 1 μg/mL for 5 hours. Collect supernatent and assay for TNFα production by standard sandwich ELISA (R&D Systems, Minneapolis, Minn.). Compound inhibition was determined relative to cells cultured with LPS alone.

Assay for Her-2 Sheddase Activity

A human breast cell cancer line BT474 (ATCC, Manassas, Va.), is seeded at 2×10⁴ cells/well in 100 μL in a 96 well plate (Costar/Corning VWR, NJ) in RPMI 1640 media (In Vitrogen, Carlsbad, Calif.) containing 10% fetal bovine serum (Hyclone, Lenexa, Kans.), and incubated overnight at 37° C., 5% CO₂. The following morning media is removed and fresh media is added back at 100 μL/well. Compounds are added at appropriate concentrations and the cells are incubated for 72 hour at 37° C., 5% CO₂. Supernatants are then removed and either tested immediately or stored at −20° C. until testing can be performed. Supernatants are tested at a 1/20 dilution for inhibition of Her-2 sheddase by commercial ELISA (Oncogene Research, San Diego, Calif.)). Compound inhibition was determined relative to cells cultured alone.

ADAM and MMP In Vitro Assays

Except for ADAM17 and MT1-MMP, all recombinant human MMPs and ADAMs were obtained from R&D Systems (Minneapolis, Minn.). Their catalog numbers are as following: MMP1 (901-MP), MMP2 (902-MP), MMP3 (513-MP), MMP7 (907-MP), MMP8 (908-MP), MMP9 (911-MP), MMP10 (910-MP), MMP12 (919-MP), MMP13 (511-MM), ADAM9 (939-AD), and ADAM10 (936-AD). MT1-MMP was obtained from US Biological (Swampscott, Mass.) with a catalog number of M2429. Porcine ADAM17 was purified in house from porcine spleen.

Fluorogenic Peptide substrate, (7-methoxycoumarin-4-yl)acetyl-Pro-Leu-Gly-Leu-(3-[2,4-dinitrophenyl]-L-2,3-diaminopropionyl)-Ala-Arg-NH₂, was obtained from R&D Systems with a catalog number of ES001. It was used as substrate for MMP1, MMP2, MMP7, MMP8, MMP9, MMP12, MMP13, and MT1-MMP assays. Fluorogenic Peptide substrate, (7-methoxycoumarin-4-yl)acetyl-Arg-Pro-Lys-Pro-Val-Glu-Nva-Trp-Arg-Lys(2,4-dinitrophenyl)-NH₂, was obtained from R&D Systems with a catalog number of ES002. It was used as substrate for MMP3 and MMP10 assays. Fluorogenic Peptide substrate, (7-methoxycourmarin-4-yl)-acetyl-Pro-Leu-Ala-Gln-Ala-Val-(3-[2,4-dinitrophenyl]-L-2,3-diaminopropionyl)-Arg-Ser-Ser-Ser-Arg-NH₂, was obtained from R&D Systems with a catalog number of ES003. It was used as substrate for ADAM9, ADAM10, and ADAM17 assays.

Assay Buffer Conditions: In general, assay buffer condition was chosen based on obtaining optimal enzymatic activities. The specific assay buffer conditions are summarized as following. For MMP1, MMP2, MMP3, MMP7, and MMP12, the assay buffer contains 50 mM Tricine, 10 mM NaCl, 10 mM CaCl₂, 1.0 mM ZnCl₂, pH 7.4. For MMP8 and MMP13, the assay buffer contains 50 mM Tricine, 10 mM NaCl, 10 mM CaCl₂, 1.0 mM ZnCl₂, 0.001% Brij35, pH 7.4. For MMP9 and MMP10, the assay buffer contains 50 mM Tris-HCl, 150 mM NaCl, 10 mM CaCl₂, 0.001% Brij35, pH 7.5. For MT1-MMP, the assay buffer contains 100 mM Tris-HCl, 100 mM NaCl, 10 mM CaCl₂, 0.001 Brij35, pH 7.5. For ADAM9, the assay buffer contains 25 mM Tris, 2.5 μM ZnCl2, and 0.001% Brij35, 0.1 mg/mL BSA, pH 9.0. For ADAM10, the assay buffer contains 25 mM Tris, 2.5 μM ZnCl₂, and 0.005% Brij35, pH 9.0. For ADAM17, the assay buffer contains 25 mM Tris, 2.5 μM ZnCl₂, and 0.001% Brij35, pH 9.0.

To activate MMP enzymes, 10 or 20 μg of lyophilized Pro-MMPs were dissolved in 100 μL of water. 100 mM p-aminophenylmercuric acetate (APMA) stock in DMSO was added to Pro-MMPs to give 1.0 mM final concentration. Pro-MMPs were incubated with APMA at 37° C. for a period time specified below. For MMP1, MMP7, and MMP8, the incubation time was 1 hour. For MMP10 and MMP13, the incubation time was 2 hours. For MMP3 and MMP9, the incubation time was 24 hours.

In general, 5 mM compound stock was prepared in DMSO. 2-Fold serial dilution starting with a specific concentration was performed to give the compound plate. 1.0 μL of compound in DMSO was transferred from compound plate to the assay plate. Enzyme solution was prepared in assay buffer with a concentration specified below. Substrate solution was prepared in assay buffer with a concentration of 20 μM. 50 μL of enzyme solution was added to the assay plate. The assay plate was incubated for 5 minutes. 50 μL of substrate solution was then added to the assay plate. Protect the plate from the light and incubate the reaction at room temperature or 37° C. for a period of time specified below. The reaction was stopped by adding 10 μL of 500 mM EDTA solution. The plate was read on a plate reader with excitation of 320 nm and emission of 405 nm. Percentage of inhibition was calculated for each concentration and IC50 value was generated from curve fitting. Specific conditions for each assay are as following: MMP1 enzyme concentration 1000 ng/mL, room temperature, 1 hour incubation; MMP2 enzyme concentration 200 ng/mL, room temperature, 1 hour incubation; MMP3 enzyme concentration 1000 ng/mL, room temperature 1 hour incubation; MMP7 enzyme concentration 100 ng/mL, room temperature 1 hour incubation; MMP8 enzyme concentration 500 ng/mL, room temperature, 2 hours incubation; MMP9 enzyme concentration 100 ng/mL, room temperature, 1 hour incubation; MMP10 enzyme concentration 1000 ng/mL, room temperature, 2 hours incubation; MMP 12 enzyme concentration 200 ng/mL, room temperature, 1 hour incubation; MMP 13 enzyme concentration 200 ng/mL, room temperature, 1.5 hours incubation; MT1-MMP enzyme concentration 200 ng/mL, room temperature, 1 hour incubation; ADAM9 enzyme concentration 4000 ng/mL, incubated at 37° C. 6 hours; ADAM10 enzyme concentration 700 ng/mL, incubated at 37° C. 6 hours; ADAM17 enzyme concentration 600 ng/mL, incubated at 37° C. 1 hours.

MMP2 assay

5 mM compound stock was prepared in DMSO. Compound plate was prepared by 2-fold dilution for 11-point curve, with highest concentration of 500 uM. 1 μL of compound in DMSO was transferred from compound plate to the assay plate. Enzyme solution was prepared in assay buffer with a concentration of 10 ng/50 μL. Substrate solution was prepared in assay buffer with a concentration of 20 μM. 50 μL of enzyme solution was added to the assay plate. The assay plate was incubated for 5 minutes. 50 μL of substrate solution was then added to the assay plate. The plate was protected from the light and the reaction was incubated at room temperature for 1 hour. The reaction was stopped by adding 10 μL of 500 mM EDTA solution. The plate was read on a plate reader with excitation of 320 nm and emission of 405 nm.

MMP3 Assay

5 mM compound stock was prepared in DMSO. Compound plate was prepared by 2-fold dilution for 11-point curve, with highest concentration of 500 uM. 1 μL of compound in DMSO was transferred from compound plate to the assay plate. Enzyme solution was prepared in assay buffer with a concentration of 50 ng/50 μL. Substrate solution was prepared in assay buffer with a concentration of 20 μM. 50 μL of enzyme solution was added to the assay plate. The assay plate was incubated for 5 minutes. Add 10 μL of 500 mM EDTA to background wells. 50 μL of substrate solution was then added to the assay plate. The plate was protected from the light and the reaction was incubated at room temperature for 1 hour. The reaction was stopped by adding 10 μL of 500 mM EDTA solution. The plate was read on a plate reader with excitation of 320 nm and emission of 405 nm.

MMP12 Assay

5 mM compound stock was prepared in DMSO. Compound plate was prepared by 2-fold dilution for 11-point curve, with highest concentration of 500 μM. 1 μL of compound in DMSO was transferred from compound plate to the assay plate. Enzyme solution was prepared in assay buffer with a concentration of 10 ng/50 μL. Substrate ((7-methoxycoumarin-4-yl)acetyl-Pro-Leu-Gly-Leu-(3-[2,4-dinitrophenyl]-L-2,3-diaminopropionyl)-Ala-Arg-NH₂) solution was prepared in assay buffer with a concentration of 20 μM. 50 μL of enzyme solution was added to the assay plate. The assay plate was incubated for 5 minutes. Add 10 μL of 500 mM EDTA in the background well. 50 μL of substrate solution was then added to the assay plate. The plate was protected from the light and the reaction incubated at room temperature for 1 hour. The reaction was stopped by adding 10 μL of 500 mM EDTA solution. The plate was read on a plate reader with excitation of 320 nm and emission of 405 nm.

ADAM10 Assay

5 mM Compound stock was prepared in DMSO. Compound plate was prepared by 2-fold dilution for 11-point curve, with highest concentration of 500 uM. 1 μL of compound in DMSO was transferred from compound plate to the assay plate. Enzyme solution was prepared in assay buffer with a concentration of 100 ng/50 μL. Substrate ((7-methoxycourmarin-4-yl)-acetyl-Pro-Leu-Ala-Gln-Ala-Val-(3-[2,4-dinitrophenyl]-L-2,3-diaminopropionyl)-Arg-Ser-Ser-Ser-Arg-NH₂) solution was prepared in assay buffer with a concentration of 20 μM. 50 μL of enzyme solution was added to the assay plate. The assay plate was incubated for 5 minutes. 50 μL of substrate solution was then added to the assay plate. The plate was protected from light and incubated at 37° C. for 4 hours. The reaction was stopped by adding 10 uL of 500 mM EDTA solution. The plate was read on a plate reader with excitation of 320 nm and emission of 405 nm.

ADAM15 Assay

ADAM15 can be assayed in a similar fashion to ADAM10 (see, e.g., Fourie et al., J. Biol Chem. 2003, 278(33), 30469-77). In brief, a fluorescence quenched peptide substrate is made by labeling one terminus with a fluorescent dye and the other terminus with a quencher dye. Cleavage of the peptide by ADAM15 can be measured by the increase in fluorescence intensity as a result of the decrease in proximity of the quencher dye to the fluorescent dye.

Compound Activity

The compounds of the present invention have IC₅₀ values less than about 20 μM for target inhibition when tested by at least one of the above in vitro assays.

### Example B

In Vivo Assay

To measure the antineoplastic activity of metalloprotease inhibitors, both estrogen dependent (MCF-7 and BT-474) and independent (MDA-MB-435) human breast cancer cell lines were used in immune compromised mouse (BALB/c nude and SCID/bg) xenograft experiments. The BT-474 tumors were from a subclone of the parental BT-474 cells from ATCC (BT-474-SC1) that were selected based on their increased tumor take and growth rates but are referred to herein as BT-474 for simplicity sake. In the BT-474 and MCF-7 tumor models, slow-release estrogen pellets (Innovative Research of America) were inserted subcutaneously (s.c.) into the flank of each mouse 24 hours prior to tumor cell inoculation. For all models, the indicated number of cells is combined with BD Matrigel™ at a 1:1 ratio immediately prior to implantation. The day after estrogen pellet implantation, 2×10⁷ BT-474 cells were injected s.c. into the upper flank of each mouse. MCF-7 tumors were generated by s.c. implantation of 5×10⁶ cells injected in similar fashion. For the MDA-MB-435 tumor cells, 2×10⁶ cells were inject s.c. into the flank of BALB/c nude mice. For all models, tumors were measured on a weekly basis and their volumes calculated using the formula [volume=(length×width²)÷2]. Once the mean tumor volume of the required number of mice reached the desired size (usually >150 mm³), they were randomized into treatment groups usually containing between 6 and 10 mice. Animals were then treated with test compound or vehicle by mini-osmotic pump implated i.p. or s.c. for 7 to 28 days to achieve the desired compound exposure—controlled by altering the pump flow rate and/or the concentration of compound inside the pumps. Tumor size and body weights (a measure of animal health) were monitored weekly. Blood samples were also drawn while the osmotic pumps were functional and plasma was separated (by centrifugation) and stored at −80° C. for later pharmacokinetic analysis.

Various modifications of the invention, in addition to those described herein, will be apparent to those skilled in the art from the foregoing description. Such modifications are also intended to fall within the scope of the appended claims. Each publication and reference, including patents, patent application, and books, cited in the present application is incorporated herein by reference in its entirety.

1. A compound of Formula I or II: 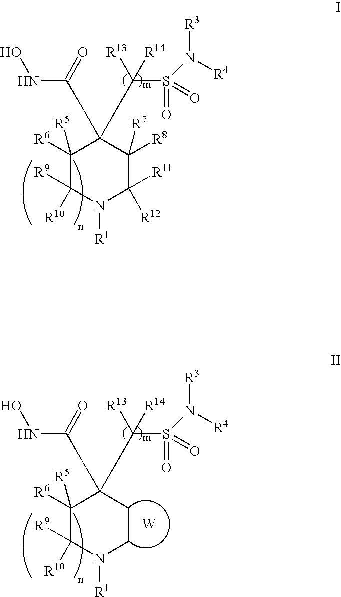
or pharmaceutically acceptable salt or prodrug thereof, wherein:
R¹ is: 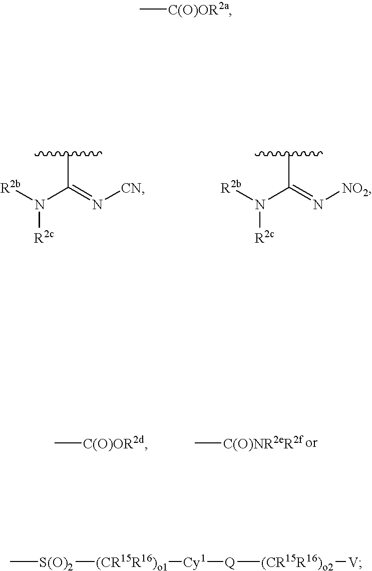
R^{2a} is:
C₂₋₁₀ alkenyl;
C₂₋₁₀ alkynyl;
—(CR¹⁷R¹⁸)ₚ₁—X—(CR¹⁷R¹⁸)ₚ₂—Y;
—(CR¹⁷R¹⁸)_{q1}—NR^{A}R^{B};
—(CR¹⁷R¹⁸)_{q2}-Z;
carbocyclyl substituted by at least one OH or C₁₋₈ haloalkoxy; or
heterocyclyl optionally substituted by 1, 2 or 3 R^{a};
R^{2b} and R^{2c} are each, independently, H, C₁₋₆ alkyl, carbocyclyl, heterocyclyl, carbocyclylalkyl or heterocyclylalkyl, each optionally substituted by 1 or 2 R^{b};
or R^{2b} and R^{2c} together with the N atom to which they are attached form a 4-14 membered heterocyclyl group optionally substituted by 1 or 2 R^{b};
R^{2d} is C₁₋₈ alkyl, carbocyclyl or heterocyclyl, each substituted by 1, 2 or 3 C₁₋₈ haloalkoxy;
R^{2e} is C₁₋₈ alkyl, carbocyclyl or heterocyclyl, each substituted by 1, 2 or 3 C₁₋₈ haloalkoxy;
R^{2f} is H, C₁₋₈ alkyl, C₃₋₇ cycloalkyl or heterocycloalkyl, wherein said C₁₋₈ alkyl, C₃₋₇ cycloalkyl or heterocycloalkyl is optionally substituted by 1 or 2 R^{c};
or R^{2e} and R^{2f} together with the N atom to which they are attached form a heterocyclyl group substituted by at least one C₁₋₈ haloalkoxy and optionally substituted by 1 or 2 R^{c};
R³ and R⁴ are each, independently, H, C₁₋₈ alkyl, C₃₋₇ cycloalkyl or heterocycloalkyl, wherein each of said C₁₋₈ alkyl, C₃₋₇ cycloalkyl or heterocycloalkyl is optionally substituted by 1 or 2 R^{d};
or R³ and R⁴ together with the N atom to which they are attached form a heterocyclyl group optionally substituted with -L-Cy² and optionally substituted with 1, 2, 3, 4 or 5 R^{e};
R⁵, R⁶, R⁷, R⁸, R⁹, R¹⁰, R¹¹ and R¹² are each, independently, H, halo, CN, NO₂, C₁₋₄ alkyl, C₁₋₄ haloalkyl, C(O)R^{w}, C(O)OR^{x}, OR^{x}, OC(O)R^{w}, NR^{y}C(O)R^{w}, SOR^{w}, SO₂R^{w} or NR^{y}R^{z};
or R⁵ and R⁶, R⁷ and R⁸, R⁹ and R¹⁰, or R¹¹ and R¹² together with the C atom to which they are attached form C═O, C═C(R¹⁹R²⁰) or a C₃₋₇ cycloalkyl or 3-7 membered heterocycloalkyl group, wherein said C₃₋₇ cycloalkyl or 3-7 membered heterocycloalkyl group is optionally substituted by 1 or 2 R^{f};
R¹³ and R¹⁴ are each, independently, H, OH, halo, C₁₋₄ alkyl, C₁₋₄ haloalkyl, C₁₋₄ alkoxy, C₁₋₄ haloalkoxy, carbocyclyl, carbocyclylalkyl, heterocyclyl or heterocyclylalkyl, wherein each of said carbocyclyl, carbocyclylalkyl, heterocyclyl or heterocyclylalkyl is optionally substituted by one or more halo, C₁₋₄ alkyl, C₁₋₄ haloalkyl, OH, C₁₋₄ alkoxy, C₁₋₄ haloalkoxy, COOH, COO(C₁₋₄ alkyl), NH₂, NH(C₁₋₄ alkyl) or N(C₁₋₄ alkyl)₂;
or R¹³ and R¹⁴ together with the C atom to which they are attached form CO, C═C(R²¹R²²), C₃₋₇ cycloalkyl or 3-7 membered heterocycloalkyl group, wherein said C₃₋₇ cycloalkyl or 3-7 membered heterocycloalkyl group is optionally substituted with 1 or 2 R^{f};
R¹⁵ and R¹⁶ are each, independently, H, halo, C₁₋₄ alkyl, C₁₋₄ haloalkyl, C₁₋₄ alkoxy or C₁₋₄ haloalkoxy, carbocyclyl, carbocyclylalkyl, heterocyclyl or heterocyclylalkyl, wherein each of said carbocyclyl, carbocyclylalkyl, heterocyclyl or heterocyclylalkyl is optionally substituted by one or more halo, C₁₋₄ alkyl, C₁₋₄ haloalkyl, OH, C₁₋₄ alkoxy, C₁₋₄ haloalkoxy, COOH, COO(C₁₋₄ alkyl), NH₂, NH(C₁₋₄ alkyl) or N(C₁₋₄ alkyl)₂;
or R¹⁵ and R¹⁶ together with the C atom to which they are attached form a 3- to 7-membered cycloalkyl or heterocycloalkyl group which is optionally substituted by one or more halo, C₁₋₄ alkyl, C₁₋₄ haloalkyl, OH, C₁₋₄ alkoxy, C₁₋₄ haloalkoxy, NH₂, COOH, COO(C₁₋₄ alkyl), NH(C₁₋₄ alkyl) or N(C₁₋₄ alkyl)₂;
R¹⁷ and R¹⁸ are each, independently, H, halo, C₁₋₄ alkyl, C₁₋₄ haloalkyl, C₁₋₄ alkoxy or C₁₋₄ haloalkoxy, carbocyclyl, carbocyclylalkyl, heterocyclyl or heterocyclylalkyl, wherein each of said carbocyclyl, carbocyclylalkyl, heterocyclyl or heterocyclylalkyl is optionally substituted by one or more halo, C₁₋₄ alkyl, C₁₋₄ haloalkyl, OH, C₁₋₄ alkoxy, C₁₋₄ haloalkoxy, COOH, COO(C₁₋₄ alkyl), NH₂, NH(C₁₋₄ alkyl) or N(C₁₋₄ alkyl)₂;
or R¹⁷ and R¹⁸ together with the C atom to which they are attached form a 3- to 7-membered cycloalkyl or heterocycloalkyl group which is optionally substituted by one or more halo, C₁₋₄ alkyl, C₁₋₄ haloalkyl, OH, C₁₋₄ alkoxy, C₁₋₄ haloalkoxy, NH₂, COOH, COO(C₁₋₄ alkyl), NH(C₁₋₄ alkyl) or N(C₁₋₄ alkyl)₂;
R¹⁹, R²⁰, R²¹, and R²² are each, independently, H, C₁₋₆ alkyl, C₂₋₆ alkenyl, C₂₋₆ alkynyl, carbocyclyl, carbocyclylalkyl, heterocyclyl, heterocyclylalkyl, CO—(C₁₋₄ alkyl), CO-(carbocyclyl), CO-(carbocyclylalkyl), or COO—(C₁₋₄ alkyl);
R^{A} and R^{B} are each, independently, H, C₁₋₆ alkyl, C₂₋₆ alkenyl, C₂₋₆ alkynyl, carbocyclyl, carbocyclylalkyl, heterocyclyl, heterocyclylalkyl, CO—(C₁₋₄ alkyl), CO-(carbocyclyl), CO-(carbocyclylalkyl), SO₂—(C₁₋₄ alkyl), SO₂-(carbocyclyl) or SO₂-(carbocyclylalkyl);
or R^{A} and R^{B} together with the N atom to which they are attached form a 3-, 4-, 5-, 6- or 7-membered heterocycloalkyl group optionally substituted by 1, 2 or 3 R^{g};
Cy¹ is absent, carbocyclyl or heterocyclyl, wherein said carbocyclyl or heterocyclyl is optionally substituted by 1, 2, 3, 4 or 5 R^{h};
Cy² is carbocyclyl or heterocyclyl, wherein said carbocyclyl or heterocyclyl is optionally substituted by 1, 2, 3, 4 or 5 Rⁱ;
L is absent, O, S, CO, C(O)O, OC(O), NRⁿ, NRⁿS(O)ᵣ, NRⁿC(O), NRⁿC(O)O, NRⁿC(O)NRⁿ, S(O)ᵣNRⁿ, NRⁿS(O)ᵣ, NRⁿS(O)NRⁿ, C₁₋₁₀ alkylene substituted with one or more R^{m} or C₂₋₁₀ alkenylene substituted with one or more R^{m};
Q is O, S or NR^{o};
V is H, carbocyclyl or heterocyclyl, wherein said carbocyclyl or heterocyclyl is optionally substituted by 1, 2, 3, 4 or 5 R^{j};
W is fused carbocyclyl or fused heterocyclyl;
X is O or S;
Y is H, halo, carbocyclyl or heterocyclyl, wherein said carbocyclyl or heterocyclyl is optionally substituted by 1, 2, 3, 4 or 5 R^{k};
Z is a 3-, 4-, 5-, 6- or 7-membered heterocyclyl group optionally substituted by 1, 2, 3, 4 or 5 R^{L};
R^{a}, R^{b}, R^{c}, R^{d}, R^{e}, R^{f}, R^{g}, R^{h}, Rⁱ, R^{j}, R^{k}, R^{L} and R^{wc} are each, independently T, (C₁₋₈ alkylene)-T, (C₂₋₈ alkenylene)-T, (C₂₋₈ alkynylene)-T, C(O)NR^{aa}(CR^{bb}R^{cc})ₛ-T, C(O)O(CR^{bb}R^{cc})ₛ-T, S(O)ₜ(CR^{bb}R^{cc})ₛ-T, (CR^{bb}R^{cc})ₛ—O—(CR^{bb}R^{cc})ₛ-T, OH, Cl, F, Br, I, CN, NO₂, NR^{I}R^{II}, COR^{III}, COOR^{IV}, OR^{IV}, CONR^{I}R^{II}, NR^{I}CONR^{I}R^{II}, OCONR^{I}R^{II}, NR^{I}COR^{II}, SO₂NR^{I}R^{II}, NR^{I}SO₂R^{II}, NR^{I}SO₂NR^{I}R^{II}, OSO₂NR^{I}R^{II}, S(O)ₜR^{V}, C₁₋₈ haloalkyl, carbocyclyl, heterocyclyl, carbocyclylalkyl, or heterocyclylalkyl, wherein each of said carbocyclyl, heterocyclyl, carbocyclylalkyl, and heterocylcylalkyl groups is optionally substituted by one or more C₁₋₈ alkyl, alkoxy, halo, haloalkyl, haloalkoxy, cyano, nitro, amino, alkylamino, dialkylamino, carboxy, carboxy alkyl ester, carboxy aryl ester, aminocarbonyl, alkylaminocarbonyl, dialkylaminocarbonyl, sulfonyl, aminosulfonyl, alkylaminosulfonyl, dialkylaminosulfonyl, arylsulfonyl, arylsulfinyl, alkylsulfonyl or arylsufonyl;
R^{m} is halo, C₁₋₄ alkyl, C₁₋₄ haloalkyl, OH, C₁₋₄ alkoxy, C₁₋₄ haloalkoxy, NH₂, COOH, COO(C₁₋₄ alkyl), NH(C₁₋₄ alkyl) or N(C₁₋₄ alkyl)₂;
Rⁿ is H or C₁₋₄ alkyl;
R^{o} is H, C₁₋₄ alkyl, carbocyclyl, heterocyclyl, carbocyclylalkyl, heterocyclylalkyl, C(O)—(C₁₋₄ alkyl) or C(O)-(cycloalkyl);
R^{w} is H, halo, C₁₋₄ alkyl, C₁₋₄ haloalkyl or NR^{wa}R^{wb};
R^{x} is C₁₋₄ alkyl or C₁₋₄ haloalkyl;
R^{y} and R^{z} are each, independently, H or C₁₋₄ alkyl;
or R^{y} and R^{z} together with the N atom to which they are attached form a 5-, 6-, or 7-membered heterocyclyl group;
R^{wa} and R^{wb} are each, independently, H, C₁₋₆ alkyl, carbocyclyl, heterocyclyl, carbocyclylalkyl or heterocyclylalkyl, each optionally substituted by 1 or 2 R^{wc};
or R^{wa} and R^{wb} together with the N atom to which they are attached form a 4-14 membered heterocyclyl group optionally substituted by 1 or 2 R^{wc};
T is H, C₁₋₁₀ alkyl optionally substituted with 1, 2, 3, 4 or 5 R^{bb}; C₂₋₁₀ alkenyl optionally substituted with 1, 2, 3, 4 or 5 R^{bb}, C₂₋₁₀ alkynyl optionally substituted with 1, 2, 3, 4 or 5 R^{bb}, carbocyclyl optionally substituted with 1, 2, 3, 4 or 5 R^{bb} or heterocyclyl optionally substituted with 1, 2, 3, 4 or R^{bb};
R^{aa}, R^{bb} and R^{cc} are each, independently, H, C₁₋₆ alkyl, C₂₋₆ alkenyl, C₂₋₆ alkynyl, OH, Cl, F, Br, I, CN, NO₂, NR^{I}R^{II}, COR^{III}, COOR^{IV}, OR^{IV}, CONR^{I}R^{II}, R^{I}NCONR^{I}R^{II}, OCONR^{I}R^{II}, R^{I}NCOR^{II}, SO₂NR^{I}R^{II}, NR^{I}SO₂R^{II}, NR^{I}SO₂NR^{I}R^{II}, OSO₂NR^{I}R^{II}, SOₚR^{V}, C₁₋₈ haloalkyl, carbocyclyl, heterocyclyl, carbocyclylalkyl, heterocyclylalkyl, carbocyclyloxy or heterocyclyloxy, wherein each of said carbocyclyl, heterocyclyl, carbocyclylalkyl, heterocyclylalkyl, carbocyclyloxy or heterocyclyloxy group is optionally substituted by one or more C₁₋₈ alkyl, alkoxy, halo, C₁₋₄ haloalkyl, C₁₋₈ haloalkoxy, cyano, nitro, amino, alkylamino, dialkylamino, carboxy, carboxy alkyl ester, carboxy aryl ester, aminocarbonyl, alkylaminocarbonyl, dialkylaminocarbonyl, sulfonyl, aminosulfonyl, alkylaminosulfonyl, dialkylaminosulfonyl, arylsulfonyl, arylsulfinyl, alkylsulfonyl or arylsufonyl;
R^{I} and R^{II} are each, independently, H, C₁₋₆ alkyl or carbocyclyl;
R^{III} and R^{IV} are each, independently, H, C₁₋₆ alkyl, haloalkyl, carbocyclyl, heterocyclyl, carbocyclylalkyl or heterocyclylalkyl, wherein said carbocyclyl, heterocyclyl, carbocyclylalkyl or heterocyclylalkyl are each optionally substituted by one or more halo, C₁₋₄ alkyl or C₁₋₄ alkoxy;
R^{V} is C₁₋₆ alkyl, haloalkyl, carbocyclyl or heterocyclyl;
m is 1 or 2;
n is 0, 1 or 2;
o1 is 0, 1, 2, 3, 4, 5 or 6;
o2 is 0, 1, 2, 3, 4, 5 or 6;
p1 is 1, 2, 3, 4, 5 or 6;
p2 is 1, 2, 3, 4, 5 or 6;
q1 is 1, 2, 3, 4, 5 or 6;
q2 is 1, 2, 3, 4, 5 or 6;
r is 1 or 2;
s is 1, 2, 3, 4, 5 or 6; and
t is 1 or 2.

2. The compound of claim 1 wherein R¹ is —C(O)OR^{2a}.

3. The compound of claim 2 wherein R^{2a} is C₂₋₁₀ alkenyl or C₂₋₁₀ alkynyl.

4. The compound of claim 2 wherein R^{2a} is —(CR¹⁷R¹⁸)ₚ₁—X—(CR¹⁷R¹⁸)ₚ₂—Y.

5. The compound of claim 2 wherein R^{2a} is —(CR¹⁷R¹⁸)_{q1}—NR^{A}R^{B}.

6. The compound of claim 5 wherein R^{A} and R^{B} are each, independently, H, C₁₋₆ alkyl or CO—(C₁₋₄ alkyl).

7. The compound of claim 5 wherein at least one of R^{A} and R^{B} is other than H.

8. The compound of claim 2 wherein R^{2a} is —(CR¹⁷R¹⁸)_{q2}-Z.

9. The compound of claim 8 wherein Z is a 3-, 4-, 5-, 6- or 7-membered heterocycloalkyl group optionally substituted by 1, 2, 3, 4 or 5 R^{L}.

10. The compound of claim 2 wherein R^{2a} is carbocyclyl substituted by at least one OH or C₁₋₈ haloalkoxy.

11. The compound of claim 10 wherein R^{2a} is aryl substituted by at least one OCF₃ or OCF₂CF₃.

12. The compound of claim 2 wherein R^{2a} is heterocyclyl optionally substituted by 1, 2 or 3 R^{a}.

13. The compound of claim 12 wherein R^{2a} is heterocycloalkyl optionally substituted by one or more Cl, F, Br, I, C₁₋₈ alkyl, C₁₋₈ haloalkyl, OH, C₁₋₈ alkoxy, C₁₋₈ haloalkoxy, CN, NO₂, NH₂, COOH, COO(C₁₋₄ alkyl), NH(C₁₋₄ alkyl) or N(C₁₋₄ alkyl)₂.

14. The compound of claim 1 wherein R¹ is

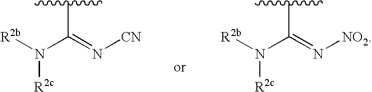

15. The compound of claim 14 wherein R^{2b} and R^{2c} are each, independently, H, C₁₋₆ alkyl, carbocyclyl, heterocyclyl, carbocyclylalkyl or heterocyclylalkyl, each optionally substituted by 1 or 2 R^{b}.

16. The compound of claim 14 wherein at least one of R^{2b} and R^{2c} is other then H.

17. The compound of claim 14 wherein R^{2b} and R^{2c} together with the N atom to which they are attached form a 4-14 membered heterocyclyl group optionally substituted by 1 or 2 R^{b}.

18. The compound of claim 1 where R¹ is —C(O)R^{2d}, —C(O)NR^{2e}R^{2f} or —S(O)₂—(CR¹⁵R¹⁶)ₒ₁-Cy¹-Q-(CR¹⁵R¹⁶)ₒ₂—V.

19. The compound of claim 1 wherein R³ and R⁴ together with the N atom to which they are attached form a heterocyclyl group optionally substituted with at least one -L-Cy² and optionally substituted with 1, 2, 3, 4 or 5 R^{e}.

20. The compound of claim 19 wherein L is O, CH₂ or absent.

21. The compound of claim 19 wherein Cy² is aryl or heteroaryl each optionally substituted by 1, 2, 3, 4 or 5 Rⁱ.

22. The compound of claim 1 wherein m is 1.

23. The compound of claim 1 wherein R⁵, R⁶, R⁷, R⁸, R⁹, R¹⁰, R¹¹ and R¹² are each, independently, H, halo, CN, NO₂, C₁₋₄ alkyl or C₁₋₄ haloalkyl.

24. The compound of claim 1 wherein R¹³ and R¹⁴ are each H.

25. The compound of claim 1 wherein n is 1.

26. The compound of claim 1 having Formula III:

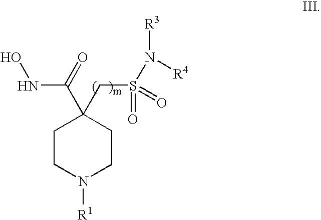

27. A compound of claim 1 selected from:
4-[4-(4-Cyano-2-methyl-phenyl)-3,6-dihydro-2H-pyridine-1-sulfonylmethyl]-4-hydroxycarbamoyl-piperidine-1-carboxylic acid-3(S)-tetrahydrofuran-3-yl ester;
4-[4-(4-Cyano-2-methyl-phenyl)-piperidine-1-sulfonylmethyl]-4-hydroxycarbamoyl-piperidine-1-carboxylic acid-3(S)-tetrahydrofuran-3-yl ester;
4-Hydroxycarbamoyl-4-(4-phenyl-3,6-dihydro-2H-pyridine-1-sulfonylmethyl)-piperidine-1-carboxylic acid-3(S)-tetrahydrofuran-3-yl ester;
4-Hydroxycarbamoyl-4-(4-phenyl-piperidine-1-sulfonylmethyl)-piperidine-1-carboxylic acid-3(S)-tetrahydrofuran-3-yl ester;
4-Hydroxycarbamoyl-4-(4-phenyl-piperazine-1-sulfonylmethyl)-piperidine-1-carboxylic acid-3(S)-tetrahydrofuran-3-yl ester;
4-[4-(3,5-Dimethyl-phenyl)-piperazine-1-sulfonylmethyl]-4-hydroxycarbamoyl-piperidine-1-carboxylic acid-3(S)-tetrahydrofuran-3-yl ester;
4-[4-(4-Cyano-2-methyl-phenyl)-piperazine-1-sulfonylmethyl]-4-hydroxycarbamoyl-piperidine-1-carboxylic acid-3(S)-tetrahydrofuran-3-yl ester;
4-Hydroxycarbamoyl-4-[4-(3-isopropyl-phenyl)-piperazine-1-sulfonylmethyl]-piperidine-1-carboxylic acid-3(S)-tetrahydrofuran-3-yl ester;
4-[4-(3,5-Dimethyl-phenyl)-3,6-dihydro-2H-pyridine-1-sulfonylmethyl]-4-hydroxycarbamoyl-piperidine-1-carboxylic acid-3(S)-tetrahydrofuran-3-yl ester;
4-[4-(4-Cyano-3,5-dimethyl-phenyl)-3,6-dihydro-2H-pyridine-1-sulfonylmethyl]-4-hydroxycarbamoyl-piperidine-1-carboxylic acid-3(S)-etrahydrofuran-3-yl ester;
4-(3,4-Dihydro-1H-isoquinoline-2-sulfonylmethyl)-4-hydroxycarbamoyl-piperidine-1-carboxylic acid-3(S)-tetrahydrofuran-3-yl ester;
4-[4-(4-Cyano-3,5-dimethyl-phenyl)-piperidine-1-sulfonylmethyl]-4-hydroxycarbamoyl-piperidine-1-carboxylic acid-3(S)-tetrahydrofuran-3-yl ester;
4-[4-(3,5-Dimethyl-phenyl)-piperidine-1-sulfonylmethyl]-4-hydroxycarbamoyl-piperidine-1-carboxylic acid-3(S)-tetrahydrofuran-3-yl ester;
4-[4-(1-Ethyl-1H-indazol-6-yl)-3,6-dihydro-2H-pyridine-1-sulfonylmethyl]-4-hydroxycarbamoyl-piperidine-1-carboxylic acid-3(S)-tetrahydrofuran-3-yl ester;
4-[4-(1-Ethyl-1H-indazol-6-yl)-piperazine-1-sulfonylmethyl]-4-hydroxycarbamoyl-piperidine-1-carboxylic acid-3(S)-tetrahydrofuran-3-yl ester;
4-[4-(1-Ethyl-1H-indazol-6-yl)-piperidine-1-sulfonylmethyl]-4-hydroxycarbamoyl-piperidine-1-carboxylic acid-3(S)-tetrahydrofuran-3-yl ester;
4-Hydroxycarbamoyl-4-[4-(3-isopropyl-phenyl)-3,6-dihydro-2H-pyridine-1-sulfonylmethyl]-piperidine-1-carboxylic acid-3(S)-tetrahydrofuran-3-yl ester;
4-(1,3-Dihydro-isoindole-2-sulfonylmethyl)-4-hydroxycarbamoyl-piperidine-1-carboxylic acid-3(S)-tetrahydrofuran-3-yl ester;
4-Hydroxycarbamoyl-4-[4-(3-isopropyl-phenyl)-piperidine-1-sulfonylmethyl]-piperidine-1-carboxylic acid-3(S)-tetrahydrofuran-3-yl ester;
4-Hydroxycarbamoyl-4-(3-phenyl-2,5-dihydro-pyrrole-1-sulfonylmethyl)-piperidine-1-carboxylic acid-3(S)-tetrahydrofuran-3-yl ester;
4-Hydroxycarbamoyl-4-(3-phenyl-pyrrolidine-1-sulfonylmethyl)-piperidine-1-carboxylic acid-3(S)-tetrahydrofuran-3-yl ester;
(3S)-Tetrahydrofuran-3-yl 4-[(hydroxyamino)carbonyl]-4-({[4-(2-methylphenyl)-3,6-dihydropyridin-1(2H)-yl]sulfonyl}methyl)piperidine-1-carboxylate;
(3S)-Tetrahydrofuran-3-yl 4-[(hydroxyamino)carbonyl]-4-({[4-(2-methylphenyl)piperidin-1-yl]sulfonyl}methyl)piperidine-1-carboxylate;
(3S)-Tetrahydrofuran-3-yl 4-[(hydroxyamino)carbonyl]-4-({[4-(2-methylphenyl)piperazin-1-yl]sulfonyl}methyl)piperidine-1-carboxylate;
(3S)-Tetrahydrofuran-3-yl 4-({[4-(2,3-dimethylphenyl)piperazin-1-yl]sulfonyl}methyl)-4-[(hydroxyamino)carbonyl]piperidine-1-carboxylate;
(3S)-Tetrahydrofuran-3-yl 4-({[4-(2-fluorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2-fluorophenyl)piperidin-1-yl]sulfonyl}methyl)-4-[(hydroxyamino)carbonyl]piperidine-1-carboxylate;
(3S)-Tetrahydrofuran-3-yl 4-({[4-(2-fluorophenyl)piperazin-1-yl]sulfonyl}methyl)-4-[(hydroxyamino)carbonyl]piperidine-1-carboxylate;
(3S)-Tetrahydrofuran-3-yl 4-[(hydroxyamino)carbonyl]-4-({[4-[2-(trifluoromethyl)phenyl]-3,6-dihydropyridin-1(2H)-yl]sulfonyl}methyl)piperidine-1-carboxylate;
(3S)-Tetrahydrofuran-3-yl 4-[(hydroxyamino)carbonyl]-4-[({4-[2-(trifluoromethyl)phenyl]piperidin-1-yl}sulfonyl)methyl]piperidine-1-carboxylate;
(3S)-Tetrahydrofuran-3-yl 4-({[4-(4-cyanophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(4-cyanophenyl)piperidin-1-yl]sulfonyl}methyl)-4-[(hydroxyamino)carbonyl]piperidine-1-carboxylate;
(3S)-Tetrahydrofuran-3-yl 4-({[4-(2-cyanophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2-cyanophenyl)piperidin-1-yl]sulfonyl}methyl)-4-[(hydroxyamino)carbonyl]piperidine-1-carboxylate;
(3S)-Tetrahydrofuran-3-yl 4-({[4-(4-fluorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(4-fluorophenyl)piperidin-1-yl]sulfonyl}methyl)-4-[(hydroxyamino)carbonyl]piperidine-1-carboxylate;
(3S)-Tetrahydrofuran-3-yl 4-({[4-(2-chlorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2-chlorophenyl)piperidin-1-yl]sulfonyl}methyl)-4-[(hydroxyamino)carbonyl]piperidine-1-carboxylate;
(3S)-Tetrahydrofuran-3-yl 4-({[4-(2-chlorophenyl)piperazin-1-yl]sulfonyl}methyl)-4-[(hydroxyamino)carbonyl]piperidine-1-carboxylate;
(3S)-Tetrahydrofuran-3-yl 4-({[4-(2,6-dichlorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2,6-dichlorophenyl)piperidin-1-yl]sulfonyl}methyl)-4-[(hydroxyamino)carbonyl]piperidine-1-carboxylate;
(3S)-Tetrahydrofuran-3-yl 4-({[4-(2,6-dichlorophenyl)piperazin-1-yl]sulfonyl}methyl)-4-[(hydroxyamino)carbonyl]piperidine-1-carboxylate;
(3S)-Tetrahydrofuran-3-yl 4-({[4-(2,4,6-trichlorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2,4,6-trichlorophenyl)piperidin-1-yl]sulfonyl}methyl)-4-[(hydroxyamino)carbonyl]piperidine-1-carboxylate;
(3S)-Tetrahydrofuran-3-yl 4-({[4-(2,4,6-trichlorophenyl)piperazin-1-yl]sulfonyl}methyl)-4-[(hydroxyamino)carbonyl]piperidine-1-carboxylate;
(3S)-Tetrahydrofuran-3-yl 4-({[4-(3-chloro-6-methylphenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(3-chloro-6-methylphenyl)piperidin-1-yl]sulfonyl}methyl)-4-[(hydroxyamino)carbonyl]piperidine-1-carboxylate;
(3S)-Tetrahydrofuran-3-yl 4-({[4-(3-chloro-6-methylphenyl)piperazin-1-yl]sulfonyl}methyl)-4-[(hydroxyamino)carbonyl]piperidine-1-carboxylate;
(3S)-Tetrahydrofuran-3-yl 4-({[4-(2,5-difluorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2,5-difluorophenyl)piperidin-1-yl]sulfonyl}methyl)-4-[(hydroxyamino)carbonyl]piperidine-1-carboxylate;
(3S)-Tetrahydrofuran-3-yl 4-({[4-(2,5-difluorophenyl)piperazin-1-yl]sulfonyl}methyl)-4-[(hydroxyamino)carbonyl]piperidine-1-carboxylate;
4-Hydroxycarbamoyl-4-(4-phenyl-3,6-dihydro-2H-pyridine-1-sulfonylmethyl)-piperidine-1-carboxylic acid tetrahydropyran-4-yl ester;
4-Hydroxycarbamoyl-4-(4-phenyl-piperidine-1-sulfonylmethyl)-piperidine-1-carboxylic acid tetrahydropyran-4-yl ester;
4-Hydroxycarbamoyl-4-(4-phenyl-3,6-dihydro-2H-pyridine-1-sulfonylmethyl)-piperidine-1-carboxylic acid 2-benzyloxyethyl ester;
4-Hydroxycarbamoyl-4-(4-phenyl-3,6-dihydro-2H-pyridine-1-sulfonylmethyl)-piperidine-1-carboxylic acid 2-methoxyethyl ester;
4-Hydroxycarbamoyl-4-(4-phenyl-piperidine-1-sulfonylmethyl)-piperidine-1-carboxylic acid 2-methoxyethyl ester;
N-Hydroxy-4-[(4-phenyl-3,6-dihydropyridin-1(2H)-yl)sulfonyl]methyl-1-[3-(trifluoromethoxy)benzoyl]piperidine-4-carboxamide;
N-Hydroxy-4-[(4-phenyl-3,6-dihydropyridin-1(2H)-yl)sulfonyl]methyl-1-[4-(trifluoromethoxy)benzoyl]piperidine-4-carboxamide;
Prop-2-yn-1-yl 4-[(hydroxyamino)carbonyl]-4-[(4-phenyl-3,6-dihydropyridin-1(2H)-yl)sulfonyl]methylpiperidine-1-carboxylate;
Prop-2-yn-1-yl 4-[(hydroxyamino)carbonyl]-4-[(4-phenyl-3,6-dihydropyridin-1(2H)-yl)sulfonyl]methylpiperidine-1-carboxylate;
(3S)-Tetrahydrofuran-3-yl 4-({[4-(4-cyano-2-methylphenyl)piperidin-1-yl]sulfonyl}methyl)-4-[(hydroxyamino)carbonyl]piperidine-1-carboxylate;
1-[(Z)-Azetidin-1-yl(cyanoimino)methyl]-N-hydroxy-4-{[(4-phenyl-3,6-dihydropyridin-1(2H)-yl)sulfonyl]methyl}piperidine-4-carboxamide;
Tetrahydro-2H-pyran-4-yl 4-({[4-(4-cyano-2-methylphenyl)piperidin-1-yl]sulfonyl}methyl)-4-[(hydroxyamino)carbonyl]piperidine-1-carboxylate;
[(2S)-1-Methylpyrrolidin-2-yl]methyl 4-[(hydroxyamino)carbonyl]-4-{[(4-phenyl-3,6-dihydropyridin-1(2H)-yl)sulfonyl]methyl}piperidine-1-carboxylate;
((2S)-1-Methylpyrrolidin-2-yl)methyl 4-({[4-(4-cyano-2-methylphenyl)piperidin-1-yl]sulfonyl}methyl)-4-[(hydroxyamino)carbonyl]piperidine-1-carboxylate;
((2R)-Pyrrolidin-2-yl)methyl 4-({[4-(4-cyano-2-methylphenyl)piperidin-1-yl]sulfonyl}methyl)-4-[(hydroxyamino)carbonyl]piperidine-1-carboxylate;
(2S)-Pyrrolidin-2-ylmethyl 4-({[4-(4-cyano-2-methylphenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2-cyanophenyl)piperazin-1-yl]sulfonyl}methyl)-4-[(hydroxyamino)carbonyl]piperidine-1-carboxylate;
(3S)-Tetrahydrofuran-3-yl 4-({[4-(2,5-dimethylphenyl)piperazin-1-yl]sulfonyl}methyl)-4-[(hydroxyamino)carbonyl]piperidine-1-carboxylate;
(3S)-Tetrahydrofuran-3-yl 4-({[4-(2,4-dimethylphenyl)piperazin-1-yl]sulfonyl}methyl)-4-[(hydroxyamino)carbonyl]piperidine-1-carboxylate;
(3S)-Tetrahydrofuran-3-yl 4-({[4-(2,4-dichlorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(4-fluoro-2-methylphenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(4-cyano-2-methylphenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-(2,4-difluorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-[(hydroxyamino)carbonyl]-4-({[4-(2,4,6-trifluorophenyl)-3,6-dihydropyridin-1(2H)-yl]sulfonyl}methyl)piperidine-1-carboxylate;
(3S)-Tetrahydrofuran-3-yl 4-({[4-(2,6-difluorophenyl)-3,6-dihydropyridin-1(2H)-yl]sulfonyl}methyl)-4-[(hydroxyamino)carbonyl]piperidine-1-carboxylate;
(3S)-Tetrahydrofuran-3-yl 4-({[4-{4-[(dimethylamino)carbonyl]-2-methylphenyl}-3,6-dihydropyridin-1(2H)-yl]sulfonyl}methyl)-4-[(hydroxyamino)carbonyl]piperidine-1-carboxylate;
(3S)-Tetrahydrofuran-3-yl 4-[(hydroxyamino)carbonyl]-4-({[4-{2-methyl-4-[(methylamino)carbonyl]phenyl}-3,6-dihydropyridin-1(2H)-yl]sulfonyl}methyl)piperidine-1-carboxylate;
(3S)-Tetrahydrofuran-3-yl 4-({[4-(2,6-difluorophenyl)piperidin-1-yl]sulfonyl}methyl)-4-[(hydroxyamino)carbonyl]piperidine-1-carboxylate;
(3S)-Tetrahydrofuran-3-yl 4-({[4-(2,4-difluorophenyl)piperidin-1-yl]sulfonyl}methyl)-4-[(hydroxyamino)carbonyl]piperidine-1-carboxylate;
(3S)-Tetrahydrofuran-3-yl 4-[(hydroxyamino)carbonyl]-4-({[4-(2,4,6-trifluorophenyl)piperidin-1-yl]sulfonyl}methyl)piperidine-1-carboxylate;
(3S)-Tetrahydrofuran-3-yl 4-({[4-(4-fluoro-2-methylphenyl)piperidin-1-yl]sulfonyl}methyl)-4-[(hydroxyamino)carbonyl]piperidine-1-carboxylate;
(3S)-Tetrahydrofuran-3-yl 4-{[(4-{4-[(dimethylamino)carbonyl]-2-methylphenyl}piperidin-1-yl)sulfonyl]methyl}-4-[(hydroxyamino)carbonyl]piperidine-1-carboxylate;
(3S)-Tetrahydrofuran-3-yl 4-[(hydroxyamino)carbonyl]-4-{[(4-{2-methyl-4-[(methylamino)carbonyl]phenyl}piperidin-1-yl)sulfonyl]methyl}piperidine-1-carboxylate;
(3S)-Tetrahydrofuran-3-yl 4-[(hydroxyamino)carbonyl]-4-[({4-hydroxy-4-[2-(trifluoromethyl)phenyl]piperidin-1-yl}sulfonyl)methyl]piperidine-1-carboxylate;
(3S)-Tetrahydrofuran-3-yl 4-({[4-(2-fluorophenyl)-4-hydroxypiperidin-1-yl]sulfonyl}methyl)-4-[(hydroxyamino)carbonyl]piperidine-1-carboxylate;
(3S)-Tetrahydrofuran-3-yl 4-[({4-[4-(aminomethyl)-2-methylphenyl]piperidin-1-yl}sulfonyl)methyl]-4-[(hydroxyamino)carbonyl]piperidine-1-carboxylate;
3-Methyltetrahydrofuran-3-yl 4-[(hydroxyamino)carbonyl]-4-({[4-(2-methylphenyl)piperidin-1-yl]sulfonyl}methyl)piperidine-1-carboxylate;
4,4-Dimethyltetrahydrofuran-3-yl 4-[(hydroxyamino)carbonyl]-4-({[4-(2-methylphenyl)piperidin-1-yl]sulfonyl}methyl)piperidine-1-carboxylate;
trans-2-Hydroxycyclohexyl 4-[(hydroxyamino)carbonyl]-4-({[4-(2-methylphenyl)piperidin-1-yl]sulfonyl}methyl)piperidine-1-carboxylate;
cis-2-Hydroxycyclohexyl 4-[(hydroxyamino)carbonyl]-4-({[4-(2-methylphenyl)piperidin-1-yl]sulfonyl}methyl)piperidine-1-carboxylate;
trans-2-Hydroxycyclopentyl 4-[(hydroxyamino)carbonyl]-4-({[4-(2-methylphenyl)piperidin-1-yl]sulfonyl}methyl)piperidine-1-carboxylate;
4-Methyltetrahydro-2H-pyran-4-yl 4-[(hydroxyamino)carbonyl]-4-({[4-(2-methylphenyl)piperidin-1-yl]sulfonyl}methyl)piperidine-1-carboxylate;
1-Methylpiperidin-3-yl 4-[(hydroxyamino)carbonyl]-4-({[4-phenyl-piperidin-1-yl]sulfonyl}methyl)piperidine-1-carboxylate;
1-Methylpiperidin-3-yl 4-[(hydroxyamino)carbonyl]-4-({[4-phenyl-3,6-dihydropyridin-1(2H)-yl]sulfonyl}methyl)piperidine-1-carboxylate;
{(2S)-1-[(Benzyloxy)carbonyl]pyrrolidin-2-yl}methyl 4-({[4-(4-cyano-2-methylphenyl)-3,6-dihydropyridin-1(2H)-yl]sulfonyl}methyl)-4-[(hydroxyamino)carbonyl]piperidine-1-carboxylate;
{(2R)-1-[(benzyloxy)carbonyl]pyrrolidin-2-yl}methyl 4-({[4-(4-cyano-2-methylphenyl)-3,6-dihydropyridin-1(2H)-yl]sulfonyl}methyl)-4-[(hydroxyamino)carbonyl]piperidine-1-carboxylate;
N-Hydroxy-1-[4-(pentafluoroethoxy)benzoyl]-4-{[(4-phenyl-3,6-dihydropyridin-1(2H)-yl)sulfonyl]methyl}piperidine-4-carboxamide;
(2R)-Pyrrolidin-2-ylmethyl 4-({[4-(4-cyano-2-methylphenyl)-3,6-dihydropyridin-1(2H)-yl]sulfonyl}methyl)-4-[(hydroxyamino)carbonyl]piperidine-1-carboxylate;
(2S)-Pyrrolidin-2-ylmethyl 4-({[4-(4-cyano-2-methylphenyl)piperidin-1-yl]sulfonyl}methyl)-4-[(hydroxyamino)carbonyl]piperidine-1-carboxylate;
(2R)-Pyrrolidin-2-ylmethyl 4-({[4-(4-cyano-2-methylphenyl)piperidin-1-yl]sulfonyl}methyl)-4-[(hydroxyamino)carbonyl]piperidine-1-carboxylate;
[(2R)-1-Methylpyrrolidin-2-yl]methyl 4-({[4-(4-cyano-2-methylphenyl)-3,6-dihydropyridin-1(2H)-yl]sulfonyl}methyl)-4-[(hydroxyamino)carbonyl]piperidine-1-carboxylate;
[(2R)-1-Methylpyrrolidin-2-yl]methyl 4-({[4-(4-cyano-2-methylphenyl)piperidin-1-yl]sulfonyl}methyl)-4-[(hydroxyamino)carbonyl]piperidine-1-carboxylate;
[(2S)-1-Methylpyrrolidin-2-yl]methyl 4-[(hydroxyamino)carbonyl]-4-{[(4-phenyl-3,6-dihydropyridin-1(2H)-yl)sulfonyl]methyl}piperidine-1-carboxylate;
[(2S)-1-Methylpyrrolidin-2-yl]methyl 4-[(hydroxyamino)carbonyl]-4-{[(4-phenylpiperidin-1-yl)sulfonyl]methyl}piperidine-1-carboxylate;
(2R)-Pyrrolidin-2-ylmethyl 4-[(hydroxyamino)carbonyl]-4-({[4-(2-methylphenyl)piperidin-1-yl]sulfonyl}methyl)piperidine-1-carboxylate;
Pyrrolidin-3-yl 4-[(hydroxyamino)carbonyl]-4-({[4-(2-methylphenyl)piperidin-1-yl]sulfonyl}methyl)piperidine-1-carboxylate;
1-Methylpyrrolidin-3-yl 4-[(hydroxyamino)carbonyl]-4-{[(4-phenyl-3,6-dihydropyridin-1(2H)-yl)sulfonyl]methyl}piperidine-1-carboxylate;
Tetrahydro-2H-pyran-4-yl 4-({[4-(4-cyano-2-methylphenyl)-3,6-dihydropyridin-1(2H)-yl]sulfonyl}methyl)-4-[(hydroxyamino)carbonyl]piperidine-1-carboxylate;
4-({[4-(4-Cyano-2-methylphenyl)-3,6-dihydropyridin-1(2H)-yl]sulfonyl}methyl)-N-hydroxy-1-[3-(pentafluoroethoxy)benzoyl]piperidine-4-carboxamide;
N-Hydroxy-1-[3-(pentafluoroethoxy)benzoyl]-4-{[(4-phenyl-3,6-dihydropyridin-1(2H)-yl)sulfonyl]methyl}piperidine-4-carboxamide; and
N-hydroxy-1-[3-(pentafluoroethoxy)benzoyl]-4-{[(4-phenylpiperidin-1-yl)sulfonyl]methyl}piperidine-4-carboxamide.

28. A composition comprising a compound of claim 1 and a pharmaceutically acceptable carrier.

29. A method for modulating activity of a metalloprotease comprising contacting said metalloprotease with a compound of claim 1.

30. The method of claim 29 wherein said modulating is inhibiting.

31. The method of claim 29 wherein said metalloprotease is an ADAM.

32. The method of claim 31 wherein said metalloprotease is ADAM10, ADAM15 or ADAM 17.

33. The method of claim 29 wherein said metalloprotease is a matrix metalloprotease.

34. A method for treating a disease associated with metalloprotease activity in a mammalian subject, the method comprising administering to said mammalian subject a therapeutically effective amount of a compound of claim 1.

35. The method of claim 34 wherein said disease is arthritis, cancer, cardiovascular disorders, skin disorders, inflammation or allergic conditions.

36. The method of claim 35 wherein said disease is cancer.

37. The method of claim 36 wherein said disease is breast cancer.

38. A method for treating a disease associated with ADAM activity in a mammalian subject, the method comprising administering to said mammalian subject a therapeutically effective amount of a compound of claim 1.

39. A method for treating a disease associated with MMP activity in a mammalian subject, the method comprising administering to said mammalian subject a therapeutically effective amount of a compound of claim 1.
